SEQUENCE LISTING (32 sequences)

SEQ ID NO: 1 - DNA, 21 bp, Homo Sapien
agggcctctcaaggcctcctc

SEQ ID NO: 2 - DNA, 23 bp, Homo Sapien
agttggagtctgtaggacttggc

SEQ ID NO: 3 - DNA, 38 bp, Homo Sapien
ctaggatatcctcgagcaagccgtggtgggaggacgag

SEQ ID NO: 4 - DNA, 41 bp, Homo Sapien
ctaggatatcaagcttttcagtcctccagcacactgcgcag

SEQ ID NO: 5 - DNA, 40 bp, Homo Sapien
ctaggatatcctcgagcgctcccaaccaagctctcttgag

SEQ ID NO: 6 - DNA, 46 bp, Homo Sapien
ctaggatatcaagcttttcatttggagaattcgatgatcaactcac

SEQ ID NO: 7 - DNA, 22 bp, Homo Sapien
gaccacaacggtttccctctag

SEQ ID NO: 8 - DNA, 22 bp, Homo Sapien
gttattgctcagcggtggcagc

SEQ ID NO: 9 - protein, 509 aa, Homo Sapien
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 10 - protein, 1210 aa, Homo Sapien [sequence omitted]

SEQ ID NO: 11 - DNA, 29 bp, Homo Sapien
catggagaatgggtgtctagtggattttc

SEQ ID NO: 12 - DNA, 29 bp, Homo Sapien
gaaaatccactagacacccattctccatg

SEQ ID NO: 13 - DNA, 39 bp, Homo Sapien
ctaggatatcccatgggcaagccgtggtgggaggacgag

SEQ ID NO: 14 - DNA, 38 bp, Homo Sapien
ctaggatatcccatggctcccaaccaagctctcttgag

SEQ ID NO: 15 - DNA, 96 bp, Homo Sapien
gatcctccgaaaccatggctcgaggcggccgcaagcttgatatcccaacgaccgaaaacctgtattttcagggccatcaccatcaccatcactagc

SEQ ID NO: 16 - DNA, 96 bp, Homo Sapien
agctgctagtgatggtgatggtgatggccctgaaaatacaggttttcggtcgttgggatatcaagcttgcggccgcctcgagccatggtttcggag

SEQ ID NO: 17 - DNA, 85 bp, Homo Sapien
ggtacccatgggaaggcgccacatcgttcggaagcgcacgctgcggaggctgctgcaggagagggagcttgtggagcctcttaca

SEQ ID NO: 18 - DNA, 75 bp, Homo Sapien
ggatcaagcttttcaatgcattctttcatccccctgaatgacaaggtagcgctgggggtctcgggccattttgga

SEQ ID NO: 19 - protein, 393 aa, Homo Sapien [sequence omitted]

SEQ ID NO: 20 - DNA, 42 bp, Homo Sapien
cgcgcggatccatgcccaagaagaagccgacgcccatccagc

SEQ ID NO: 21 - DNA, 44 bp, Homo Sapien
cgtagctcgagtcaggtaccggcagcgtgggttggtgtgctggg

SEQ ID NO: 22 - DNA, 44 bp, Homo Sapien; identical to SEQ ID NO: 21

SEQ ID NO: 23 - DNA, 33 bp, Homo Sapien
gcgagatcagcatctccatggagcacatggatg

SEQ ID NO: 24 - DNA, 31 bp, Homo Sapien
catggatggtgggtgcttggatcaagtgctg

SEQ ID NO: 25 - DNA, 31 bp, Homo Sapien
gggagatcaaactctccgattttggggtcag

SEQ ID NO: 26 - DNA, 31 bp, Homo Sapien
gggagatcaaactcgccgattttggggtcag

SEQ ID NO: 27 - DNA, 46 bp, Homo Sapien
cgggcagctaattgacgacatggccaacgacttcgtgggaacaagg

SEQ ID NO: 28 - DNA, 46 bp, Homo Sapien; identical to SEQ ID NO: 27

SEQ ID NO: 29 - DNA, 31 bp, Homo Sapien
gagctgctgtttggatcccaggtggaaggag

SEQ ID NO: 30 - DNA, 35 bp, Homo Sapien
ggattttgtgaataagtccttaataaagaaccctg

SEQ ID NO: 31 - DNA, 35 bp, Homo Sapien
ggattttgtgaataagatgttaataaagaaccctg

SEQ ID NO: 32 - DNA, 40 bp, Homo Sapien
gacttcgcaggctggctctcctccaccattgggcttaacc

(19) United States
(12) Patent Application Publication
(10) Pub. No.: US 2005/0084905 A1
(43) Pub. Date: Apr. 21, 2005

(54) Identification of kinase inhibitors

(76) Inventors: John C. Prescott, San Francisco, CA (US); Andrew Braisted, (US); Joelle Morrow, San Francisco, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/666,927

(22) Filed: Sep. 17, 2003

Related U.S. Application Data
(63) Continuation-in-part of application No. 10/394,322, filed on Mar. 20, 2003.
(60) Provisional application No. 60/366,892, filed on Mar. 21, 2002.

(51) Int. Cl. G01N 33/53; C12Q 1/48

(52) U.S. Cl. 435/7.1; 435/15

(57) ABSTRACT

The invention concerns the identification of protein kinase inhibitors that preferentially bind to the inactive conformation of a target protein kinase. The inhibitors are identified by locking the target protein kinase in an inactive conformation, and using Tethering to identify inhibitors preferentially targeting the inactive conformation.

### CROSS-REFERENCE TO RELATED APPLICATION

This application claims the benefit of U.S. patent application Ser. No. 10/394,322, filed Mar. 20, 2003 which in turn claims the benefit of U.S. Provisional Patent Application No. 60/366,892, filed Mar. 21, 2002, which are incorporated herein by reference.

### BACKGROUND OF THE INVENTION

Field of the Invention

The growth and differentiation of eukaryotic cells is regulated by a complex web of signal transduction pathways. Precise regulation of these pathways allows cells to respond to extracellular stimuli such as hormones, neurotransmitters, and stress as they proliferate and differentiate into specific tissues. Protein phosphorylation, a regulatory mechanism common to all eukaryotic cells, plays a central role in this signal transduction web. First discovered as a regulatory mechanism nearly fifty years ago, protein phosphorylation is very likely the most important mechanism for regulation of signal transduction in mammalian cells. It is therefore not surprising that protein kinases, enzymes that catalyze the transfer of the γ-phosphatase group of ATP to the oxygen atom of the hydroxyl group of serine, threonine or tyrosine residues in peptides and polypeptides, comprise one of the largest protein superfamilies. Indeed, with the complete sequencing of the human genome, it has been asserted that there are exactly 508 genes encoding human protein kinases, including 58 receptor tyrosine kinases and 32 nonreceptor tyrosine kinases.

Kinases and Cancer

Cancer consists of a variety of diseases characterized by abnormal cell growth. Cancer is caused by both internal and external factors that cause mutations in the genetic material of the cells. Cancer causing genetic mutations can be grouped into two categories, those that act in a positive manner to increase the rate of cell growth, and those that act in a negative manner by removing natural barriers to cell growth and differentiation. Mutated genes that increase the rate of cell growth and differentiation are called oncogenes, while those that remove natural barriers to growth are called tumor suppressor genes.

The first oncogene identified encoded the Src tyrosine kinase. Src is a key regulator of signal transduction in many different cell types. Present inside nearly all human cells in an inactive state, Src is poised to respond to extracellular signals from a variety of sources. Once triggered by a stimulus, Src is transformed into an active state in which it phosphorylates tyrosine residues on a number of effector proteins. While the tyrosine kinase activity of Src is tightly regulated in normal cells, mutations can occur which transform the enzyme into a constitutively active state. It was one such mutation, identified over 25 years ago, that gave Src the dubious honor of being known as the first oncogene. There are now about 30 tumor suppressor genes and over 100 oncogenes known, about 20% of which encode tyrosine kinases. The disregulation of such central regulators of cell growth and differentiation has disastrous consequences for the cell.

Kinase Inhibitors

Protein kinases play a crucial role not only in signal transduction but also in cellular proliferation, differentiation, and various regulatory mechanisms. The casual role that many protein kinases play in oncogenesis has made them exciting targets for the development of novel anti-cancer chemotherapies. The conserved and extremely well characterized nature of the ATP binding pocket has made it the most common, and most successful, target for kinase inhibition. Thus, libraries containing ATP (and purine) mimetics have been generated and screened against large panels of kinases in the hope of finding those rare pharmacophores that can outcompete ATP, thereby blocking kinase activity.

However, this approach has at least two major shortcomings. First, these inhibitors must compete directly with ATP for their binding site. ATP, which is used by thousands of cellular enzymes, is present in cells in very high concentration. Therefore, kinase inhibitors that act in a strictly ATP competitive manner must bind to their target kinase with extremely high affinity. Second, the high structural conservation surrounding the ATP binding pocket (also known as the purine binding pocket) makes it difficult to design inhibitors that show specificity for one kinase over another. Given these two criticisms, it is perhaps not surprising that after twenty years of research there are only twelve small molecule tyrosine kinase inhibitors in clinical trials. All of these inhibitors compete directly with ATP for the ATP binding pocket, all bind this pocket extremely tightly, and all show varying degrees of specificity for their target kinase.

A possible exception is the small molecule kinase inhibitors, Gleevec™ (Novartis), a phenylamino-pyrimidine derivative, which binds the purine pocket of Abl tyrosine kinase. This compound shows unique properties that suggest that its mode of action is somewhat unusual. While this compound was approximately a thousand fold less potent than most kinase inhibitor clinical candidates when assayed in biochemical assays, it did not lose as much potency as most inhibitors did when tested in cells, suggesting that it is not competing directly with cellular ATP for binding to Abl. Co-crystallization studies have shown that Gleevec™ does indeed occupy the purine pocket of the Abl kinase, but it does so only when the kinase is in an inactive conformation, with the amino-terminal and carboxy-terminal lobes twisted such that the catalytic triad of lysine and two aspartic acids is not properly aligned to accept ATP or to catalyze the phosphate transfer reaction. Therefore, Gleevec™ makes use of the proven small molecule druggability of the purine pocket without directly competing against ATP, which binds to the inactive conformation with much lower affinity.

It would be desirable to develop protein kinase inhibitors that do not compete directly with ATP for binding to the active conformation of the ATP binding pocket of the target protein kinase. It would be further desirable to design fast, reliable, high-throughput screening assays for identifying such inhibitors. It would also be desirable to lock the ATP binding pocket of protein kinases in an inactive conformation in order to facilitate the design of such screening assays and the identification of protein kinase inhibitors with unique properties, such as increased specificity.

### SUMMARY OF THE INVENTION

In one aspect, the invention concerns a method for identifying a ligand binding to an inactive conformation of a target protein kinase, comprising
(a) contacting the inactive conformation of the target protein kinase, which contains or is modified to contain a reactive group at or near a binding site of interest, with one or more ligand candidates capable of covalently bonding to the reactive group thereby forming a kinase-ligand conjugate; and
(b) detecting the formation of the kinase-ligand conjugate and identifying the ligand present in the kinase-ligand conjugate.

The kinase and the ligand candidate preferably form a disulfide bond to yield a kinase-ligand conjugate. In this embodiment, the kinase and the ligand candidate(s) can be contacted in the presence of a reducing agent, such as 2-mercaptoethanol or cysteamine.

The ligand candidates may be small molecules, and may be less than 1500 daltons, preferably less than 1000 daltons, more preferably less than 750 daltons, even more preferably less than 500 daltons, most preferably less than 250 daltons in size.

In another aspect, the invention concerns a method for identifying a ligand that binds to the inactive conformation of a target protein kinase, comprising
(a) obtaining the inactive conformation of the target protein kinase comprising an —SH group, masked —SH group, or activated —SH group;
(b) combining the inactive conformation of the target protein kinase with one or more ligand candidates wherein said ligand candidates each comprises a disulfide bond;
(c) forming a kinase-ligand conjugate wherein at least one ligand candidate binds to the inactive conformation of the target protein kinase under disulfide exchange conditions, and
(d) detecting the formation of the kinase-ligand conjugate and identifying the ligand present in the conjugate.

In another aspect, the invention concerns a method for identifying a ligand that binds to the inactive conformation of a target protein kinase, comprising
(a) obtaining the inactive conformation of the target protein kinase comprising an —SH group, masked —SH group, or activated —SH group;
(b) combining in a mixture the inactive conformation of the target protein kinase with a plurality of ligand candidates that are each capable of forming a disulfide bond with the —SH group, masked —SH group, or activated —SH group thereby forming at least one kinase-ligand conjugate;
(c) analyzing the mixture by mass spectrometry; and
(d) detecting the most abundant kinase-ligand conjugate that is formed and identifying the ligand thereon.

In yet another aspect, the invention concerns a method for identifying ligands binding to an inactive conformation of a target protein kinase, comprising
(a) contacting the inactive conformation of the protein kinase having a first and a second binding site of interest and containing or modified to contain a nucleophile at or near the first site of interest with a plurality of ligand candidates, the candidates having a functional group reactive with the nucleophile, under conditions such that a reversible covalent bond is formed between the nucleophile and a candidate that has affinity for the first site of interest, to form a kinase-first ligand complex;
(b) identifying the first ligand from the complex of (a);
(c) designing a derivative of the first ligand identified in (b) to provide a small molecule extender (SME) having a first functional group reactive with the nucleophile on the kinase and a second functional group reactive with a second ligand having affinity for the binding second site of interest;
(d) contacting the SME with the kinase to form a kinase-SME complex, and
(e) contacting the kinase-SME complex with a plurality of second ligand candidates, the candidates having a functional group reactive with the SME in said kinase-SME complex, wherein a candidate that has affinity for the second binding site of interest on the kinase forms a reversible covalent bond with said kinase-SME complex, whereby a second ligand is identified.

In a still further aspect, the invention concerns a method for identifying ligands binding to an inactive conformation of a target protein kinase, comprising
(a) screening a library of ligand candidates with a kinase-ligand conjugate formed by the covalent bonding of the inactive conformation of a kinase comprising a first reactive functionality with a compound that comprises (1) a second reactive functionality and (2) a chemically reactive group, wherein the second reactive functionality of the compound reacts with the first reactive functionality of the inactive conformation of the target protein kinase to form a first covalent bond such that the kinase-ligand conjugate contains a free chemically reactive group, under conditions wherein at least one member of the library forms a second covalent bond with the kinase-ligand conjugate; and
(b) identifying a further ligand that binds covalently to the chemically reactive group of the kinase-ligand conjugate.

### BRIEF DESCRIPTION OF THE DRAWINGS

FIG. 1A is a schematic illustration of one embodiment of Tethering. A thiol-containing protein is reacted with a plurality of ligand candidates. A ligand candidate that possesses an inherent binding affinity for the target is identified and a ligand is made comprising the identified binding determinant (represented by the circle) that does not include the disulfide moiety.

FIG. 1B is a schematic representation of one embodiment of Tethering where an extender comprising a first and second functionality is used. As shown, a target that includes a thiol is contacted with an extender comprising a first functionality -LG that is capable of forming a covalent bond with the reactive thiol and a second functionality second functionality -SPG that is capable of forming a disulfide bond. A target-extender covalent complex is formed which is then contacted with a plurality of ligand candidates. The extender provides one binding determinant (circle) and the ligand candidate provides the second binding determinant (square) and the resulting binding determinants are linked together to form a conjugate compound.

FIG. 2 illustrates the mass spectrometer profile of purified EGFR1 kinase domain. FIG. 2A is purified EGFR1 in the active conformation. FIG. 2B is purified EGFR1 in the inactive conformation. FIGS. 2C-E) are purified EGFR1 in the inactive conformation following incubation with C) cystamine, D) a quinazoline extender, and E) the quinazoline extender and cystamine.

FIG. 3 is a schematic depicting the progression from the design and synthesis of a purine pocket extender, through a library screen, and ending with a soluble MEK1 inhibitor. The portion of the molecule that binds to the adaptive binding pocket is indicated by a circle. The MEK1 construct used in each of these successive steps, either the S150C screening mutant or wild type, are indicated on the left.

FIG. 4 is a specificity profile of three inhibitors that were derived from Tethering that inhibit MEK1 with IC₅₀'s of 80 nM, 50 nM, and 10 nM respectively. ATP concentrations were varied such that the assays were run at or near the Kₘ for ATP for the various kinases: 10 mM ATP (IKKb, MEK1, MKK4); 15 mM ATP (Aurora-A, CaMKII, CSK, FGFR3, Zap-70); 45 mM ATP (CDK2/cyclinA, c-RAF, JNK1a1, PKCa, Yes); 50 mM ATP (MEK1 inactive conformation); 90 mM ATP (SAPK2a); 155 mM ATP (MAPK2, PKBa); and 200 mM ATP (cSRC, IR).

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and Constituents of Signaling Pathways and their Chemistry, New Science Press Ltd. 2002, provide one skilled in the art with a general guide to many of the terms used in the present application.

The term “protein kinase” is used to refer to an enzyme that catalyzes the transfer of the γ-phosphoryl group of ATP (ATP-Mg²⁺ complex) to the oxygen atom of the hydroxyl group of serine, threonine or tyrosine residues in peptides and polypeptides (substrates).

The term “tyrosine kinase” is used to refer to an enzyme that catalyzes the transfer of the γ-phosphoryl group from an ATP-Mg²⁺ complex to the oxygen atom of the hydroxyl group of tyrosine residues in another protein (substrate).

The term “serine-threonine kinase” is used to refer to an enzyme that catalyzes the transfer of the γ-phosphoryl group from an ATP-Mg²⁺ complex to the oxygen atom of the hydroxyl group of serine/threonine residues in another protein (substrate).

The term “dual specificity kinase” is used to refer to kinases that have the unusual ability to phosphorylate both tyrosine and serine/threonine residues of targeted protein substrates, and typically function at pivotal positions in signal transduction pathways.

The term “phosphoryl donor” refers to an ATP-Mg²⁺ complex, where the divalent Mg²⁺ ion helps orient the nucleotide and shields the negative charges on its β- and γ phosphoryl groups, reducing electrostatic repulsion of attacking nucleophiles.

The term “phosphoacceptor” is used to refer to an atom with a free electron pair that serves as the nucleophile to attack ATP-Mg²⁺ (e.g., the oxygen atom of the deprotonated hydroxyl groups of the side chains of Ser, Thr, or Tyr residues in a protein). For example, in the substrates of tyrosine kinases, the phosphoacceptor is the oxygen atom of the deprotonated hydroxyl group of the side chain of a tyrosine (Tyr) residue.

The term “activation loop” is used to a highly variable segment in protein kinases, situated between the DFG motif and the APE motif that contains the sites of activating phosphorylation in nearly all protein kinases.

The terms “catalytic loop” and “catalytic domain” are used interchangeably and refer to residues in conserved protein kinase motif VIb, which contains an invariant aspartic acid (Asp) residue that serves as the catalytic base in phosphotransfer and a nearly invariant arginine (Arg) residue, that makes electrostatic contact with phosphorylated residues in the activation loop, leading to the catalytically active state of the kinase.

The term “APE motif” is used to refer to the residues in conserved protein kinase motif VIII, which contains an invariant glutamic acid (Glu) residue that caps a small helix and an invariant proline (Pro) residue that terminates the same helix.

The term “DFG motif” is used to refer to the residues in conserved protein kinase motif VII, which contains an invariant aspartic acid (Asp) residue that helps mold the active site by forming hydrogen-bonds with the invariant lysine (Lys) in motif II and an invariant asparagine (Asn) residue in motif VIb, thus helping stabilize the conformation of the catalytic loop.

The term “inactive conformation,” as used herein, refers to a catalytically inactive state of the protein. For example, a protein kinase is in an inactive conformation when the activation loop is not phosphorylated. A kinase is said to be locked in an inactive conformation when the kinase assumes the inactive conformation and does not phosphorylate its intended substrate.

An “inactivation site” on a protein kinase as used herein is any site on the kinase that, when occupied by a ligand, adversely affects the formation of the active conformation or otherwise impairs the kinase's ability to phosphorylate its intended substrate. Alternatively, an inactivation site when referring to an amino acid residue on the kinase is a residue that is directly or indirectly involved in the phosphorylation of the activation loop, and/or in the presentation or transfer of the γ-phosphoryl group of ATP (ATP-Mg²⁺ complex) to the substrate of the protein kinase, and/or in any other interaction between the protein kinase and its substrate.

A kinase inhibitor binds “preferentially” to an inactive conformation of a target kinase, if its binding affinity to the inactive conformation is at least two fold of its binding affinity to the active conformation.

A “ligand” as defined herein is an entity which has an intrinsic binding affinity for the target. The ligand can be a molecule, or a portion of a molecule which binds the target. The ligands are typically small organic molecules which have an intrinsic binding affinity for the target molecule, but may also be other sequence-specific binding molecules, such as peptides (D-, L- or a mixture of D- and L-), peptidomimetics, complex carbohydrates or other oligomers of individual units or monomers which bind specifically to the target. The term also includes various derivatives and modifications that are introduced in order to enhance binding to the target. Ligands that inhibit a biological activity of a target molecule are called “inhibitors” of the target.

A “ligand candidate” is a compound that has a moiety that is capable of forming a covalent bond with a reactive group on a target kinase or with a reactive group on a target-kinase-SME covalent complex. A ligand candidate becomes a ligand of a target once it is determined that it has an intrinisc binding affinity for the target.

The term “inhibitor” is used in the broadest sense and includes any ligand that partially or fully blocks, inhibits or neutralizes a biological activity exhibited by a target protein kinase. In a similar manner, the term “agonist” is used in the broadest sense and includes any ligand that mimics a biological activity exhibited by a target protein kinase.

A “binding site of interest” on a target protein kinase as used herein is a site to which a specific ligand binds. Typically, the molecular interactions between the ligand and the binding site of interest on the target are non-covalent, and include hydrogen bonds, van der Waals interactions and electrostatic interactions. On target protein kinases, the binding site of interest broadly includes the amino acid residues involved in binding of the target to a molecule with which it forms a natural complex in vivo or in vitro.

“Small molecules” are usually less than about 10 kDa molecular weight, and include but are not limited to synthetic organic or inorganic compounds, peptides, (poly)nucleotides, (oligo)saccharides and the like. Small molecules specifically include small non-polymeric (i.e. not peptide or polypeptide) organic and inorganic molecules. Many pharmaceutical companies have extensive libraries of such molecules, which can be conveniently screened by using the extended tethering approach of the present invention. Preferred small molecules have molecular weights of less than about 1000 Da, more preferably about 500 Da, and most preferably about 250 Da.

The phrase “Small Molecule Extender” (SME) as used herein refers to a small organic molecule having a molecular weight of from about 75 to about 1,500 daltons and having a first functional group reactive with a nucleophile or electrophile on a protein kinase target and a second functional group reactive with a ligand candidate or members of a library of ligand candidates. Preferably, the first functional group on one end of the SME is reactive with a nucleophile on a protein kinase (capable of forming an irreversible or reversible covalent bond with such nucleophile), and the reactive group at the other end of the SME is a free or protected thiol or a group that is a precursor of a free or protected thiol.

The phrase “reversible covalent bond” as used herein refers to a covalent bond which can be broken, preferably under conditions that do not denature the target. Examples include, without limitation, disulfides, Schiff-bases, thioesters, and the like.

The term “reactive group” with reference to a ligand is used to describe a chemical group or moiety providing a site at which a covalent bond with the ligand candidates (e.g. members of a library or small organic compounds) may be formed. Thus, the reactive group is chosen such that it is capable of forming a covalent bond with members of the library against which it is screened.

The phrases “modified to contain” and “modified to possess” are used interchangeably, and refer to making a mutant, variant or derivative of the target, or the reactive nucleophile or electrophile, including but not limited to chemical modifications. For example, in a protein one can substitute an amino acid residue having a side chain containing a nucleophile or electrophile for a wild-type residue. Another example is the conversion of the thiol group of a cysteine residue to an amine group.

The term “reactive nucleophile” as used herein refers to a nucleophile that is capable of forming a covalent bond with a compatible functional group on another molecule under conditions that do not denature or damage the target. The most relevant nucleophiles are thiols, alcohols, and amines. Similarly, the term “reactive electrophile” as used herein refers to an electrophile that is capable of forming a covalent bond with a compatible functional group on another molecule, preferably under conditions that do not denature or otherwise damage the target. The most relevant electrophiles are imines, carbonyls, epoxides, aziridines, sulfonates, and hemiacetals.

A “first binding site of interest” on a target protein kinase refers to a site that can be contacted by at least a portion of the SME when it is covalently bound to the reactive nucleophile or electrophile. The first binding site of interest may, but does not have to possess the ability to form a bond with the SME.

The phrases “group reactive with the nucleophile,” “nucleophile reactive group,” “group reactive with an electrophile,” and “electrophile reactive group,” as used herein, refer to a functional group, e.g. on the SME, that can form a covalent bond with the nucleophile/electrophile on the target protein kinase under conditions that do not denature or otherwise damage the target.

The term “protected thiol” as used herein refers to a thiol that has been reacted with a group or molecule to form a covalent bond that renders it less reactive and which may be deprotected to regenerate a free thiol.

The phrase “adjusting the conditions” as used herein refers to subjecting a target protein kinase, such as a tyrosine kinase, to any individual, combination or series of reaction conditions or reagents necessary to cause a covalent bond to form between the ligand and the target, such as a nucleophile and the group reactive with the nucleophile on the SME, or to break a covalent bond already formed.

The term “covalent complex” as used herein refers to the combination of the SME and the target, e.g. target protein kinase which is both covalently bonded through the nucleophile/electrophile on the target with the group reactive with the nucleophile/electrophile on the SME, and non-covalently bonded through a portion of the small molecule extender and the first binding site of interest on the target.

The phrase “exchangeable disulfide linking group” as used herein refers to the library of molecules screened with the covalent complex displaying the thiol-containing small molecule extender, where each member of the library contains a disulfide group that can react with the thiol or protected thiol displayed on the covalent complex to form a new disulfide bond when the reaction conditions are adjusted to favor such thiol exchange.

The phase “highest affinity for the second binding site of interest” as used herein refers to the molecule having the greater thermodynamic stability toward the second site of interest on the target protein kinase that is preferentially selected from the library of disulfide-containing library members.

“Functional variants” of a molecule herein are variants having an activity in common with the reference molecule.

“Active” or “activity” means a qualitative biological and/or immunological property.

The term amino acid “alteration” includes amino acid substitutions, deletions, and/or insertions.

B. Detailed Description

In one aspect, the present invention provides a method for locking a protein kinase in an inactive conformation. In another aspect, the invention concerns the identification of inhibitors that preferentially bind to the inactive conformation of a target protein kinase.

Protein Kinases

Protein kinases are enzymes that catalyze the transfer of the γ-phosphoryl group of ATP (ATP-Mg²⁺ complex) to the oxygen atom of the hydroxyl group of serine, threonine or tyrosine residues in peptides and polypeptides (substrates). Protein kinases play a crucial role in signal transduction, cellular proliferation, differentiation, and various regulatory mechanisms. About 3% of the total coding sequences within the human genome encode protein kinases.

While there are many different subfamilies within the broad grouping of protein kinases, they all share a common feature; they all act as ATP phosphotransferases. It is, therefore, not surprising that protein kinases share a very high degree of structural similarity in the region where the ATP is bound, the ATP binding pocket (which is also known as the purine binding pocket). Structural analysis of many protein kinases shows that the catalytic domain, responsible for the phosphotransfer activity, is very highly conserved. This domain is comprised of two lobes that are connected by a flexible hinge region. The amino-terminal lobe is comprised of a single alpha helix and five beta sheets, while the carboxy-terminal lobe is comprised of a four alpha helix bundle and a flexible loop called the activation loop. The ATP binding pocket is formed at the interface between these two lobes. There are several highly conserved residues, including an invariant catalytic triad consisting of a single lysine and two aspartic acids. The lysine of this catalytic triad is responsible for properly positioning the γ-phosphate of ATP with the hydroxyl group of the residue in the substrate to which it is transferred (phosphoacceptor residue), while the first aspartic acid acts as a general base catalyst in the phosphotransfer reaction. Strikingly, these three crucial residues span the two lobes of the catalytic domain. Furthermore, the two aspartic acid residues within the catalytic triad are separated from each other by a second flexible region called the activation loop. To allow the phosphotransfer reaction, the structure of a substrate must conform to the geometric constraints, surface electrostatics, and other features of the active site of the corresponding protein kinase. In turn, substrate binding can induce structural changes in a kinase that stimulate its catalytic activity. In particular, for enzyme—substrate interactions, residues within the activation loop and the catalytic loop need to be made available to make contacts with side chains in a substrate. Outside the conserved motifs crucial for catalytic activity (such as the ATP binding site), there are sequence differences in both loops that are critical for substrate recognition.

Structural States of Kinases and Regulation of Kinase Activity

Proper regulation of protein kinase activity in a cell is critical, and kinases in a resting cell generally exist in an inactive conformation. In this inactive conformation, the catalytic triad may be oriented in a manner that will not catalyze phosphate transfer, the substrate binding cleft may be occluded by the flexible activation loop, or both. Relative to the active conformation, the amino- and carboxy-terminal lobes in the inactive conformation may be opened up with resultant widening the active site cleft, twisted with resultant tortioning of the active site cleft, or both. Only when cells are confronted with specific stimuli do these kinases transition to a catalytically active conformation. Transition to the active conformation almost invariably involves phosphorylation of a residue in the activation loop, and subsequent formation of a salt bridge with a conserved arginine immediately adjacent to the catalytic aspartic acid. The resultant rearrangement of the activation loop, stabilized by this newly formed salt bridge, stabilizes a catalytically active conformation characterized by: proper amino- and carboxy-terminal domain orientation, proper orientation of the γ-phosphate of ATP to allow for phosphoryl transfer, opening of the substrate binding site, and a favorable electrostatic environment for the aspartic acid mediated base catalysis.

While a common function dictates that the structure at the catalytic center is highly conserved among kinases in the active conformation, this is not the case with kinases in the inactive conformation. In fact, structural studies of the active and inactive forms of kinases reveal that kinases that have highly conserved active site architectures when in the active conformation show considerable structural diversity in the same region when they are in the inactive conformation. This is particularly true of a region immediately adjacent to the ATP binding site that has been termed the adaptive binding region. For example, Gleevec binds to the ATP binding pocket of the Abl kinase but only when it is in the inactive form. More importantly, the bulk of Gleevec binds to the adaptive binding pocket that is only revealed when Abl kinase is in the inactive form. Thus, specifically targeting the inactive form of the kinase provides a path for mitigating many of the difficulties in developing kinase inhibitors as drugs.

An important protein kinase target for drug development is the Tyr kinase EGFR1 (Ullrich et al., Nature 309:418-425 (1984); SwissProt accession code P00533). EGFR1, a validated target for chemotherapeutics, is a cell surface receptor that contains an extracellular ligand binding domain and an intracellular tyrosine kinase domain. It is a key regulator of cell growth, survival, proliferation, and differentiation in epithelial cells. The binding of a number of ligands activates EGFR1, including EGF, TGF-α, amphiregulin, β-cellulin, and epiregulin. Ligand binding leads to receptor dimerization, autophosphorylation at a number of tyrosine residues including Tyr845 in the activation loop, and subsequent recruitment pf substrate proteins and stabilization of the active conformation of the kinase domain. EGFR1, in this activated state, phosphorylates a variety of downstream targets to propagate the extracellular stimulus of ligand binding to the eventual transcriptional upregulation of a variety of growth regulatory genes and resultant cell proliferation. In normal cells, EGFR1 regulates cell growth in a tightly controlled manner. However, overexpression of EGFR1 has been observed in a large number of tumor types, including breast, bladder, colon, lung, squamous cell head and neck, ovarian, and pancreatic cancers. A clear role for EGFR1 upregulation in the initiation and progression of a variety of cancers has lead to an intense search for therapeutics that inhibit signal transduction via EGFR1.

Another important protein kinase target for drug development is the dual specificity kinase MEK1 (Seger et al., J. Biol. Chem. 267: 25628-31 (1992); Swiss Prot accession code Q02750). It is the central kinase in the mitogen activated Ras→Raf→MEK→ERK signal transduction cascase (also referred to as the MEK→ERK pathway). Conditional activation of this pathway transmits mitogenic and cell survival signals from a number of growth factors and receptors, including EGFR, VEGFR, PDGFR and FGFR. Overexpression or consitutive activation of these same growth factors and receptors in tumors correlates with a poor prognosis in cancer patients.

Further validation of MEK1 as a general cancer therapeutic target comes from the development of two specific MEK1 inhibitors. The first, PD98059, is a specific, albeit relatively insoluble, MEK1 inhibitor. Though not a therapeutic candidate, this compound has been used in over 2,500 publications validating the Ras→Raf→MEK→ERK pathway as a critical pathway in transformed cells, and confirming that inhibition of this pathway is sufficient to reverse the transformed phenotype of cells that have upregulated this pathway (e.g., cells transformed with an activated Ras mutant). The second, PD184352 (also known as CI-1040), is a specific MEK1 inhibitor currently in Phase II trials for use as a therapeutic in a variety of solid tumors. Preclinical and Phase I clinical data have clearly demonstrated that the MEK→ERK pathway can be inhibited in vivo, that inhibition of this pathway does not cause general toxicity, and that inhibition of this pathway correlates with tumor regression in multiple mouse xenograft cancer models.

In addition, the MEK→ERK pathway generally confers resistance to apoptosis. Thus, it is believed that cancers with increased MEK→ERK signaling will be more resistant to chemotherapy-induced apoptosis, and inhibition of MEK1 activity will increase the sensitivity of these cancers to traditional chemotherapeutics. In studies in acute and chronic myelogeneous leukemic cell lines, the MEK1 inhibitors PD98059 and PD184352 induced apoptosis in tumor cell lines in a manner that directly correlated with the level of ERK activation. As predicted, these MEK1 inhibitors acted synergistically with a variety of chemotherapeutic cytotoxins, including ara-C, cisplatin, and paclitaxel.

Another important family of protein kinases is the Src family. First of all, the Src family kinases are well validated casual agents in a variety of cancers. Second, no current small molecule therapeutics effectively targets Src kinases in humans. Finally, Src family kinases are the best structurally characterized of all tyrosine kinases.

A representative member of this family, the Tyr kinase Lck (Perlmutter et al., J. Cell. Biochem. 38:117-126 (1988); Swiss Prot acession code P06239), is a cytosolic tyrosine kinase, which is expressed primarily in T-cells where it is centrally involved in transducing a signal from the T-cell receptor (TCR). Lck is found associated when the inner plasma membrane where it phosphorylates the CD3 and zeta chains of the TCR in response to antigenic stimulation, initiating a cascade of signal transduction events that eventually result in a clonal proliferation of the stimulated T-cell. Thus, Lck is well known as a therapeutic target for immunological disorders, such as graft versus host disease. However, Lck is also validated cancer therapeutic target. In humans, some neuroblastomas and non-Hodgkin's lymphomas show chromosomal abnormalities and translocations in the region of the Lck gene. In at least one case that has been molecularly characterized, the “derivative I chromosome” translocation focuses the transcriptional regulatory region of the beta T-cell receptor gene with the coding sequence of Lck, resulting in increased levels of Lck kinase in patients with T-cell acute lymphoblastic leukemia, much like the Philadelphia Chromosome translocation which upregulates Abl expression causing CML.

In addition to their value as therapeutic targets, Src family kinases are extremely well characterized structurally. Crystal coordinates are publicly available for three family members, hematopoietic cell kinase (Hck), Src, and Lck, covering both the active and the inactive conformational. Furthermore, Lck is known to express well in baculovirus and to crystallize readily.

Other illustrative examples of kinase targets include but are not limited to:
Ser/Thr kinase AKT1 (Jones et al., PNAS 88: 4171-4175 (1991); Swiss Prot accession code P31749);
Ser/Thr kinase AKT2 (Jones et al., Cell Regul. 2(12): 1001-1009 (1991); Swiss Prot accession code P31751);
Ser/Thr kinase AKT3 (Brodbeck et al., J. Biol. Chem. 274(14): 9133-9136 (1999); Swiss Prot acession code Q9Y243);
Tyr kinase BLK (Islam et al., J. Immunol. 154(3): 1265-1272 (1995);Swiss Prot acession code P51451);
Tyr kinase BTK (Vetrie et al., Nature 361: 226-233 (1993); Swiss Prot accession code Q06187);
Ser/Thr kinase CDK1 (Lee et al., Nature 327: 31-35 (1987); Swiss Prot accession code P06493);
Ser/Thr kinase CDK2 (Elledge et al., EMBO J. 10(9): 2653-2659 (1991); Swiss Prot accession code P24941);
Ser/Thr kinase CDK3 (Meyerson et al., EMBO J. 11(8): 2909-2917 (1992); Swiss Prot accession code Q00526);
Ser/Thr kinase CDK4 (Hanks et al., PNAS 84: 388-392 (1987); Swiss Prot accession code P11802);
Ser/Thr kinase CDK5 (Meyerson et al., EMBO J. 11(8): 2909-2917 (1992); Swiss Prot accession code Q00535);
Ser/Thr kinase CDK6 (Meyerson et al., EMBO J. 11(8): 2909-2917 (1992); Swiss Prot accession code Q00534);
Ser/Thr kinase CDK7 (Tassan et al, J. Cell Biol. 127(2): 467-478 (1994); Swiss Prot accession code P50613);
Ser/Thr kinase CDK8 (Tassan et al., PNAS 92(19): 8871-8875 (1995); Swiss Prot accession code P49336);
Ser/Thr kinase CDK9 (Grana et al., PNAS 91: 3834-3838 (1994); Swiss Prot accession code P50750);
Tyr kinase CSK (Brauninger et al., Oncogene 8(5): 1365-1369 (1993); Swiss Prot accession code P41240);
Tyr kinase ERB2 (Semba et al., PNAS 82: 6497-6501 (1985); Swiss Prot accession code P04626);
Tyr kinase ERB4 (Plowman et al., PNAS 90(5): 1746-1750 (1993); Swiss Prot accession code Q15303);
Ser/Thr kinase ERK1 (Charest et al., Mol. Cell. Biol. 13(8): 4679-4690 (1993); Swiss Prot accession code P27361);
Ser/Thr kinase ERK2 (Owaki et al., Biochem. Biophys. Res. Commun. 182(3): 1416-1422 (1992); Swiss Prot accession code P28482);
Ser/Thr kinase ERK3 (Zhu et al., Mol. Cell. Biol. 14(12): 8202-8211 (1994); Swiss Prot accession code Q16659);
Ser/Thr kinase ERK4 (Gonzalez et al., FEBS Lett. 304: 170-178 (1992); Swiss Prot accession code P31152);
Ser/Thr kinase ERK5 (Zhou et al., J. Biol. Chem. 270(21): 12665-12669 (1995); Swiss Prot accession code Q13164);
Ser/Thr kinase ERK6 (Lechner et al., PNAS 93(9): 4355-4359 (1996); Swiss Prot accession code P53778);
Tyr kinase FAK1 (Whitney et al., DNA Cell Biol. 12(9): 823-830 (1993); Swiss Prot accession code Q05397);
Tyr kinase FGFR1 (Isacchi et al., Nucleic Acids Res. 18(7): 1906 (1990); Swiss Prot accession code P 11362);
Tyr kinase FGFR2 (Houssaint et al., PNAS 87(20): 8180-8184 (1990); Swiss Prot accession code P21802);
Tyr kinase FGFR3 (Keegan et al., PNAS 88(4): 1095-1099 (1991); Swiss Prot accession code P22607);
Tyr kinase FGFR4 (Partanen et al., EMBO J. 10(6): 1347-1354 (1991); Swiss Prot accession code P22455);
Tyr kinase FYN (Semba et al., PNAS 83: 5459-5463 (1986); Swiss Prot accession code P06241);
Tyr kinase HCK (Quintrell et al., Mol. Cell. Biol. 7(6): 2267-2275 (1987); Swiss Prot accession code P08631);
Ser/Thr kinase IKK-a (Regnier et al., Cell 90(2): 373-383 (1997); Swiss Prot accession code 015111);
Ser/Thr kinase IKK-b (Woronicz et al., Science 278: 866-869 (1997); Swiss Prot accession code 014920);
Ser/Thr kinase IKK-e (Nagase et al., DNA Res. 2(4): 167-174 (1995); Swiss Prot accession code Q14164);
Tyr kinase JAK1 (Wilks et al., Mol. Cell. Biol. 11: 2057-2065 (1991); Swiss Prot accession code P23458);
Tyr kinase JAK2 (Saltzman et al., Biochem. Biophys. Res. Commun. 246(3): 627-633 (1998); Swiss Prot accession code 060674);
Tyr kinase JAK3 (Kawamura et al., PNAS 91: 6374-6378 (1994); Swiss Prot accession code P52333);
Ser/Thr kinase JNK1 (Derijard et al., Cell 76: 1025-1037 (1994); Swiss Prot accession code P45983);
Ser/Thr kinase JNK2 (Sluss et al., Mol. Cell. Biol. 14: 8376-8384 (1994); Swiss Prot accession code P45984);
Ser/Thr kinase JNK3 (Mohit et al., Neuron 14(1): 67-78 (1995); Swiss Prot accession code P53779);
Tyr kinase LCK (Perlmutter et al., J. Cell. Biochem. 38(2): 117-126 (1988); Swiss Prot accession code P06239);
Tyr kinase LYN (Yamanashi et al., Mol. Cell. Biol. 7(1): 237-243 (1987); Swiss Prot accession code P07948);
Ser/Thr kinase MAPK (Lee et al., Nature 372: 739-746 (1994); Swiss Prot accession code Q16539);
Ser/Thr kinase NIK (Malinin et al., Nature 385: 540-544 (1997); Swiss Prot accession code Q99558);
Ser/Thr kinase PAK1 (Ottilie et al., EMBO J. 14(23): 5908-5919 (1995); Swiss Prot accession code P50527);
Ser/Thr kinase PAK2 (Swiss Prot accession code Q13177);
Ser/Thr kinase PAK3 (Allen et al., Nat. Genet. 20(1): 25-30 (1998); Swiss Prot accession code 075914);
Ser/Thr kinase PAK4 (Abo et al., EMBO J. 17(22): 6527-6540 (1998); Swiss Prot accession code 096013);
Ser/Thr kinase PAK5 (Swiss Prot accession code Q9P286);
Tyr kinase PDGFR-a (Matsui et al, Science 243: 800-804 (1989); Swiss Prot accession code P16234);
Tyr kinase PDGFR-b (Gronwald et al., PNAS 85(10): 3435-3439 (1988); Swiss Prot accession code P09619);
Ser/Thr kinase PIM1 (Reeves et al., Gene 90(2): 303-307 (1990); Swiss Prot accession code P11309);
Ser/Thr kinase A-Raf (Beck et al, Nucleic Acids Res. 15(2): 595-609 (1987); Swiss Prot accession code P10398);
Ser/Thr kinase B-Raf (Sithanandam et al., Oncogene 5: 1775-1780 (0.1990); Swiss Prot accession code P15056);
Ser/Thr kinase C-Raf (Bonner et al, Nucleic Acids Res. 14(2): 1009-1015 (1986); Swiss Prot accession code P04049);
Tyr kinase SRC (Swiss Prot accession code P12931);
Tyr kinase SRC2 (c-FGR) (Katamine et al., Mol. Cell. Biol. 8(1): 259-266 (1988); Swiss Prot accession code P09769);
Tyr kinase STK1 (FLT3) (Small et al., PNAS 91: 459-463 (1994); Swiss Prot accession code P36888);
Tyr kinase SYK (Yagi et al., Biochem. Biophys. Res. Commun. 200(1): 28-34 (1994); Swiss Prot accession code P43405);
Tyr kinase TEC (Sato et al., Leukemia 8(10): 1663-1672 (1994); Swiss Prot accession code P42680);
Ser/Thr kinase TFGR1 (Franzen et al., Cell 75(4): 681-692 (1993); Swiss Prot accession code P36897);
Ser/Thr kinase TGFR2 (Lin et al., Cell 68(4): 775-785 (1992); Swiss Prot accession code P37173);
Tyr kinase TIE1 (Partanen et al., Mol. Cell. Biol. 12(4): 1698-1707 (1992); Swiss Prot accession code P35590);
Tyr kinase TIE2 (Ziegler et al., Oncogene 8(3): 663-670 (1993); Swiss Prot accession code Q02763);
Tyr kinase VEGFRL (Yamane et al., Oncogene 9(9): 2683-2690 (1994); Swiss Prot accession code P53767);
Tyr kinase VEGFR2 (Swiss Prot accession code P35968);
Tyr kinase VEGFR3 (Galland et al., Oncogene 8(5): 1233-1240 (1993); Swiss Prot accession code P35916);
Tyr kinase YES (Sukegawa et al., Mol. Cell. Biol. 7: 41-47 (1987); Swiss Prot accession code P07947); and,
Tyr kinase ZAP-70 (Chan et al., Cell 71: 649-662 (1992); Swiss Prot accession code P43043).
   Identification of Protein Kinase Inhibitors Preferentially Binding to the Inactive Conformation

In an important aspect, the present invention provides methods for identifying protein kinase inhibitors that specifically target kinases in the inactive conformation. There are at least three principle reasons of screening for such inhibitors: (1) the majority of kinases in a cell exist in this conformation; (2) relative to the active conformation, kinases in the inactive conformation exhibit greater structural diversity; and (3) opening and tortioning of the active site region in this conformation often results in a decreased affinity for ATP, the primary intracellular competitor for small molecule kinase inhibitors.

Traditional high throughput screening techniques detect phosphoryl transfer to a substrate molecule by an activated kinase. As such, these assays primarily detect inhibitors that bind to the active conformation of kinases and make the identification of inhibitors targeting the inactive conformation very unlikely. In contrast, the present invention provides an efficient, high-throughput method to identify kinase inhibitors that bind preferentially to the inactive conformation of protein kinases. This method includes the step of locking the protein kinase in its inactive conformation, and using Tethering to identify inhibitors specifically targeting the inactive kinase conformation.

a. Locking Kinases in an Inactive Conformation

In order to identify kinase inhibitors preferentially binding to the inactive conformation of the target kinase, according to the invention a target protein kinase is locked in a catalytically inactive conformation by introducing one or more amino acid alterations at an inactivating site such that the kinase cannot exert its kinase activity, in most cases because the alteration inhibits the phosphorylation of the activation loop. The alteration may target any site participating (directly or indirectly) in the formation of a catalytically active state of the kinase. For example, the alteration may take place at or near amino acid residues participating in the phosphorylation of the activation loop, and/or in the presentation or transfer of the γ-phosphoryl group of ATP to the substrate of the protein kinase, and/or in any other interaction between the protein kinase and its substrate. Alterations within or in the vicinity of the catalytic loop, e.g. the ATP binding site including the catalytic triad, the substrate binding channel, a cofactor binding site, if any, residues involved in hydrogen bond/acceptor interactions, and/or docking of the substrate on the tyrosine kinase are particularly preferred.

For purposes of shorthand designation of the protein kinase variants described herein, it is noted that numbers refer to the position of the altered amino acid residue along the amino acid sequences of respective wild-type protein kinases. Amino acid identification uses the single-letter alphabet of amino acids, as follows:

| | | |
|---|---|---|
| Asp | D | Aspartic acid |
| Ile | I | Isoleucine |
| Thr | T | Threonine |
| Leu | L | Leucine |
| Ser | S | Serine |
| Tyr | Y | Tyrosine |
| Glu | E | Glutamic acid |
| Phe | F | Phenylalanine |
| Pro | P | Proline |
| His | H | Histidine |
| Gly | G | Glycine |
| Lys | K | Lysine |
| Ala | A | Alanine |
| Arg | R | Arginine |
| Cys | C | Cysteine |
| Trp | W | Tryptophan |
| Val | V | Valine |
| Gln | Q | Glutamine |
| Met | M | Methionine |
| Asn | N | Asparagine |

The designation for a substitution variant herein consists of a letter followed by a number followed by a letter. The first (leftmost) letter designates the amino acid in the wild-type protein kinase. The number refers to the amino acid position where the amino acid substitution is being made, and the second (right-hand) letter designates the amino acid that is used to replace the wild-type amino acid at that position. The designation for an insertion variant consists of the letter i followed by a number designating the position of the residue in wild-type protein kinase before which the insertion starts, followed by one or more capital letters indicating, inclusively, the insertion to be made. The designation for a deletion variant consists of the letter d followed by the number of the start position of the deletion to the number of the end position of the deletion, with the positions being based on the wild-type protein kinase. Multiple alterations are separated by a comma in the notation for ease of reading them.

In one embodiment, the kinase is locked in an inactive conformation by mutating one or more residues selected from the group consisting of the invariant aspartic acid in the catalytic loop; the arginine in the catalytic loop; the invariant aspartic acid in the DFG motif; and the invariant lysine in motif II. In preferred embodiments, one or more of these residues are substituted by an alanine residue.

Illustrative examples of kinase mutants where the invariant aspartic acid residue in the catalytic loop is mutated to X (wherein X denotes any amino acid residue other than aspartic acid) include any combination of the following:
D274X AKT1; D275X AKT2; D271X AKT3; D359X BLK; D521X BTK; D128X CDK1; D127X CDK2; D127X CDK3; D140X CDK4; D126X CDK5; D145X CDK6; D137X CDK7; D151X CDK8; D149X CDK9; D314X CSK; D837X EGFR1; D845X ERB2; D843X ERB4; D166X ERK1; D149X ERK2; D152X ERK3; D149X ERK4; D181X ERK5; D153X ERK6; D546X FAK1; D623X FGFR1; D626X FGFR2; D617X FGFR3; D612X FGFR4; D389X FYN; D381X HCK; D144X IKK-a; D145X IKK-b; D135X IKK-e; D991X JAK1; D976X JAK2; D949X JAK3; D151X JNK1; D151X JNK2; D189X JNK3; D363X Lck; D366X LYN; D150X MAPK; D190X MEK1; D515X NIK; D389X PAK1; D368X PAK2; D387X PAK3; D440X PAK4; D568X PAK5; D818X PDGFR-a; D826X PDGFR-b; D167X PIM1; D429X A-Raf; D575X B-Raf; D468X C-Raf; D388X SRC; D382X SRC2; D811X STK1; D494X SYK; D489X TEC; D333X TGFR1; D379X TGFR2; D979X TIE1; D964X TIE2; D1022X VEGFR1; D1028X VEGFR2; D1037X VEGFR3; D386X YES; D461X ZAP-70.

Illustrative examples of kinase mutants where the arginine residue in the catalytic loop is mutated to X (wherein X denotes any amino acid residue other than arginine) include any combination of the following:
R273X AKT1; R274X AKT2; R270X AKT3; R358X BLK; R520X BTK; R127X CDK1; R126X CDK2; R126X CDK3; R139X CDK4; R125X CDK5; R144X CDK6; R136X CDK7; R150X CDK8; R148X CDK9; R313X CSK; R836X EGFR1; R844X ERB2; R842X ERB4; R165X ERK1; R148X ERK2; R151X ERK3; R148X ERK4; R180X ERK5; R152X ERK6; R545X FAK1; R622X FGFR1; R625X FGFR2; R616X FGFR3; R611X FGFR4; R388X FYN; R380X HCK; R143X IKK-a; R144X IKK-b; R134X IKK-e; R990X JAK1; R975X JAK2; R948X JAK3; R150X JNK1; R150X JNK2; R188X JNK3; R362X Lck; R365X LYN; R149X MAPK; R189X MEK1; R514X NIK; R388X PAK1; R367X PAK2; R386X PAK3; R439X PAK4; R567X PAK5; R817X PDGFR-a; R825X PDGFR-b; R166X PM1; R428X A-Raf; R574X B-Raf; R467X C-Raf; R387X SRC; R381X SRC2; R810X STK1; R493X SYK; R488X TEC; R322X TGFR1; R378X TGFR2; R978X TIE1; R963X TIE2; R1021 VEGFR1; R1027X VEGFR2; R1036X VEGFR3; R395X YES; R460X ZAP-70.

Illustrative examples of kinase mutants where the invariant aspartic acid in the DFG motif is mutated to X (wherein X denotes any amino acid residue other than aspartic acid) include any combination of the following:
D292X AKT1; D293X AKT2; D289X AKT3; D377X BLK; D539X BTK; D146X CDK1; D145X CDK2; D145X CDK3; D158X CDK4; D144X CDK5; D163X CDK6; D155X CDK7; D173X CDK8; D167X CDK9; D332X CSK; D855X EGFR1; D863X ERB2; D861X ERB4; D184X ERK1; D167X ERK2; D171X ERK3; D168X ERK4; D199X ERK5; D171X ERK6; D564X FAK1; D641X FGFR1; D644X FGFR2; D635X FGFR3; D630X FGFR4; D407X FYN; D399X HCK; D165X IKK-a; D166X IKK-b; D157X IKK-e; D1009X JAK1; D994X JAK2; D967X JAK3; D169X JNK1; D169X JNK2; D207X JNK3; D381X Lck; D384X LYN; D168X MAPK; D208X MEK1; D534X NIK; D407X PAK1; D386X PAK2; D405X PAK3; D458X PAK4; D586X PAK5; D836X PDGFR-a; D844X PDGFR-b; D186X PIM1; D447X A-Raf; D593X B-Raf; D486X C-Raf; D406X SRC; D400X SRC2; D829X STK1; D512X SYK; D507X TEC; D351X TGFR1; D397X TGFR2; D997X TIEL; D982X TIE2; D1040X VEGFR1; D1046X VEGFR2; D1055X VEGFR3; D414X YES; D479X ZAP-70.

Illustrative examples of kinase mutants where the invariant lysine in motif II is mutated to X (wherein X denotes any amino acid residue other than lysine) include:
K179X AKT1; K181X AKT2; K177X AKT3; K268X BLK; K430X BTK; K33X CDK1; K33X CDK2; K33X CDK3; K35X CDK4; K33X CDK5; K43X CDK6; K41X CDK7; K52X CDK8; K48X CDK9; K222X CSK; K745X EGFR1; K753X ERB2; K751X ERB4; K71X ERK1; K54X ERK2; K49X ERK3; K49X ERK4; K83X ERK5; K56X ERK6; K454X FAK1; K514X FGFR1; K517X FGFR2; K508X FGFR3; K503X FGFR4; K298X FYN; K290X HCK; K44X IKK-a; K44X IKK-b; K38X IKK-e; K896X JAK1; K882X JAK2; K855X JAK3; K55X JNK1; K55X JNK2; K93X JNK3; K272X Lck; K274X LYN; K53X MAPK; K97X MEK1; K429X NIK; K299X PAK1; K228X PAK2; K297X PAK3; K350X PAK4; K478X PAK5; K627X PDGFR-a; K634X PDGFR-b; K67X PIM1; K336X A-Raf; K482X B-Raf; K375X C-Raf; L297X SRC; K291X SRC2; K644X STK1; K402X SYK; K398X TEC; K232X TGFR1; K277X TGFR2; K870X TIE1; K855X TIE2; K862X VEGFR1; K868X VEGFR2; K879X VEGFR3; K305X YES; K369 ZAP-70.

It will be appreciated that two or more of the foregoing or similar mutations can be combined to produce inactive kinase variants. Protein kinase variants comprising two or more of the above-listed mutations in any combination, including double, triple and quadruple mutants having mutations other than inactivating mutations described above, are specifically within the scope herein.

Those skilled in the art are well aware of various recombinant, chemical, synthesis and/or other techniques that can be routinely employed to modify a protein kinase of interest such that it possesses a desired number of free thiol groups that are available for covalent binding to a ligand candidate comprising a free thiol group. Such techniques include, for example, site-directed mutagenesis of the nucleic acid sequence encoding the target protein kinase. Particularly preferred is site-directed mutagenesis using polymerase chain reaction (PCR) amplification (see, for example, U.S. Pat. No. 4,683,195 issued 28 Jul. 1987; and Current Protocols In Molecular Biology, Chapter 15 (Ausubel et al., ed., 1991). Other site-directed mutagenesis techniques are also well known in the art and are described, for example, in the following publications: Ausubel et al., supra, Chapter 8; Molecular Cloning: A Laboratory Manual., 2^{nd} edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., PNAS 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London_{—}SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

The nucleic acid encoding the desired kinase mutant is then inserted into a replicable expression vector for further cloning or expression. Expression and cloning vectors are well known in the art and contain a nucleic acid sequence that enables the vector to replicate in one or more selected host cells. The selection of an appropriate vector will depend on 1) whether it is to be used for DNA amplification or for DNA expression, 2) the size of the DNA to be inserted into the vector, and 3) the host cell to be transformed with the vector. Each vector contains various components depending on its function (amplification of DNA or expression of DNA) and the host cell for which it is compatible. The vector components generally include, but are not limited to, one or more of the following: a signal sequence, an origin of replication, one or more marker genes, an enhancer element, a promoter, and a transcription termination sequence. Suitable expression vectors, for use in combination with a variety of host cells, are well known in the art and are commercially available.

The protein kinase mutants can be produced in prokaryotic or eukaryotic host cells, including bacterial hosts, such as E. coli, eukaryotic microbes, such as filamentous fungi or yeast, and host cells derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and corresponding permissive insect host cells, e.g. cells from Spodoptera frugiperda, Aedes aegypti, Aedes albopictus, Drosophila melanogaster, and Bombyx mori have been identified. A variety of viral strains for transfection of insect host cells are publicly available, including for example variants of Autographa California NPV and Bombyx mori NPV strains. Further host cells include vertebrate cells. Examples of suitable mammalin host cell lines include, without limitation, human embryonic kidney cell line 293, Chinese hamster ovary (CHO) cells, etc.

Host cells are transformed with the expression or cloning vectors encoding the desired protein kinase mutants, and cultured in conventional nutrient media modified as appropriate for inducing promoters, selecting transformants, or amplifying the genes encoding the desired sequences.

b. Tethering

According to the present invention, the protein kinases locked in inactive conformation are used to screen for inhibitors preferentially binding to the inactive conformation by using Tethering. This approach differs significantly from the conventional drug discovery route that is based on the synthesis of large organic compound libraries, and subsequent screening, usually for inhibitory activity against the target protein kinase. In Tethering, small, drug-like fragments (monophores) containing or modified to contain a moiety capable of forming a disulfide bond are tested for binding activity to the desired kinase. These monophores are then used to synthesize conjugates of fragments that bind in non-overlapping sites to generate molecules that no longer require the disulfide for binding. The linking or merging of multiple fragments effectively results in the combination of individual binding energies, plus a favorable entropic term due to the high local concentration of the second fragment once the first fragment is bound, yielding dissociation constants at levels similar to a typical medicinal chemistry starting point. In quantitative terms, this means that two fragments, each having ˜mM dissociation constants (K_{d}) can be combined to form a molecule having a ˜μM K_{d}. This “screen then link” strategy is much more efficient than the traditional approach, allowing a much larger survey of chemical diversity space than is achievable by screening even the largest compound libraries.

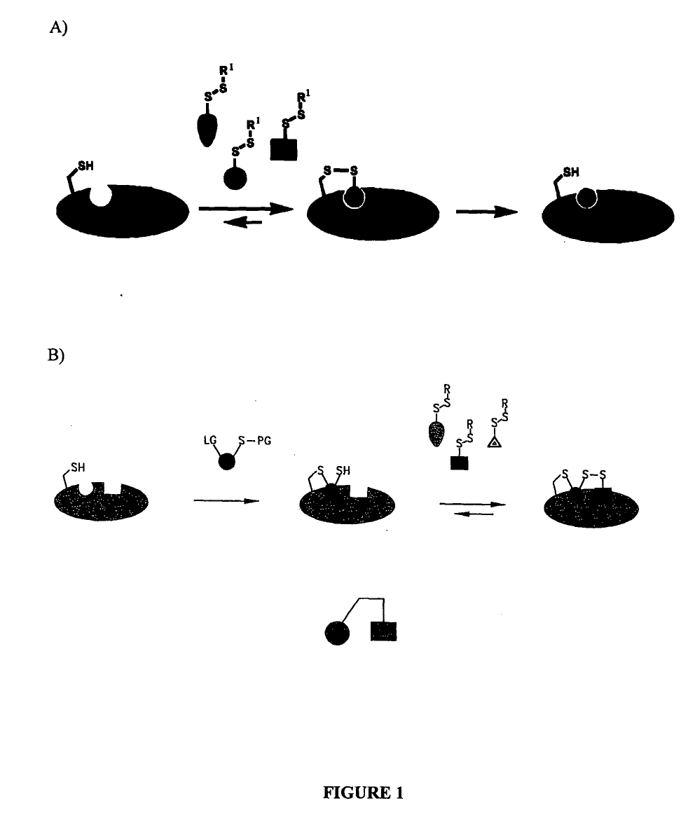

In a preferred embodiment, molecules binding to the target protein kinase locked in an inactive conformation are identified using Tethering recently reported by Erlanson et al., PNAS 97:9367-9372 (2000). This strategy is suitable for rapid and reliable identification of small soluble drug fragments that bind with low affinity to a specifically targeted site on a protein or other macromolecule, using an intermediary disulfide linker and is illustrated in FIG. 1A. According to a preferred embodiment of this approach, a library of disulfide-containing molecules is allowed to react with a cysteine-containing target protein under partially reducing conditions that promote rapid thiol exchange. If a molecule has even weak affinity for the target protein, the disulfide bond linking the molecule to the target protein will be entropically stabilized. The disulfide-bonded fragments can then be identified by a variety of methods, including mass spectrometry (MS), and their affinity improved by traditional approaches upon removal of the disulfide tether. See also PCT Publication Nos. WO 00/00823 and WO 03/046200, the entire disclosures of which are hereby expressly incorporated by reference.

Briefly, according to preferred embodiments, a disulfide bond is formed between the target protein kinase molecule locked in inactive configuration and a ligand candidate to yield a target protein-ligand conjugate, and the ligand present in the conjugate is identified. Optionally, the target protein is contacted with a ligand candidate (preferably a library of ligand candidates) in the presence of a reducing agent, such as 2-mercaptoethanol, or cysteamine. Most of the library members will have little or no intrinsic affinity for the target molecule, and thus by mass action the equilibrium will lie toward the unbound target molecule. However, if a library member does show intrinsic affinity for the target molecule, the equilibrium will shift toward the target molecule, having attached to it the library member with a disulfide containing linker. If a plurality of library members have intrinsic affinity for the target molecule, than the library member having the greatest affinity for the target molecule will form the most abundant target molecule-ligand conjugate.

The target contains, or is modified to contain, free or protected thiol groups, preferably not more than about 5 thiol groups, more preferably not more than about 2 thiol groups, more preferably not more than one free thiol group. The target protein kinase of interest may be initially obtained or selected such that it already possesses the desired number of thiol groups, or may be modified to possess the desired number of thiol groups.

As noted above, in certain embodiments the kinase of interest possesses at least one naturally occurring cysteine that is amenable to Tethering. Illustrative examples of kinases that include naturally occurring cysteines that are amenable to Tethering include: CDK5 (C53); ERK1 (C183); ERK2 (C166); ERK3 (C28); FGFR1 (C488); FGFR2 (C491); FGFR3 (C482); FGFR4 (C477); MEK1 (C207); NIK (C533); PDGFR-a (C835); PDGFR-b (C843); SRC(C279); SRC2 (C273); STK1 (C828); TGFR2 (C396); VEGFR1 (C1039); VEGFR2 (C1045); VEGFR3 (C1054); YES (C287); ZAP-70 (C346).

In other embodiments, one or more amino acids are mutated into a cysteine. In general, cysteine mutants are made using the following guidelines.

Broadly, the “binding site of interest” on a particular target, such as a target protein kinase, is defined by the residues that are involved in binding of the target to a molecule with which it forms a natural complex in vivo or in vitro. If the target is a peptide, polypeptide, or protein, the site of interest is defined by the amino acid residues that participate in binding to (usually by non-covalent association) to a ligand of the target.

When the target biological molecule is an enzyme, the binding site of interest can include amino acids that make contact with, or lie within, about 4 angstroms of a bound substrate, inhibitor, activator, cofactor or allosteric modulator of the enzyme. For protein kinases, the binding site of interest includes the substrate-binding channel and the ATP binding site.

The target protein kinases either contain, or are modified to contain, a reactive residue at or near a binding site of interest. Preferably, the target kinases contain or are modified to contain a thiol-containing amino acid residue at or near a binding site of interest. In this case, after a protein kinase is selected, the binding site of interest is calculated. Once the binding site of interest is known, a process of determining which amino acid residue within, or near, the binding site of interest to modify is undertaken. For example, one preferred modification results in substituting a cysteine residue for another amino acid residue located near the binding site of interest.

The choice of which residue within, or near, the binding site of interest to modify is determined based on the following selection criteria. First, a three dimensional description of the target protein kinase is obtained from one of several well-known sources. For example, the tertiary structure of many protein kinases has been determined through x-ray crystallography experiments. These x-ray structures are available from a wide variety of sources, such as the Protein Databank (PDB) which can be found on the Internet at http://www.rcsb.org. Tertiary structures can also be found in the Protein Structure Database (PSdb) which is located at the Pittsburg Supercomputer Center at http://www.psc.com.

In addition, the tertiary structure of many proteins, and protein complexes, including protein kinases, has been determined through computer-based modeling approaches. Thus, models of protein three-dimensional conformations are now widely available.

Once the three dimensional structure of the target protein kinase is known, or modeled based on homology to a known structure, a measurement is made based on a structural model of the wild-type, or a variant form locked in an inactive configuration, from any atom of an amino acid within the site of interest across the surface of the protein for a distance of approximately 10 angstroms. Since the goal is to identify protein kinase inhibitors that preferentially bind to an inactive conformation of the target protein kinase, preferably the site(s) of interest is/are identified base upon a structural model of the protein kinase locked in an inactive conformation. The binding sites (pockets) presented by such inactive conformations are often significantly different from the binding sites (pockets) present on the wild-type structure. Variants of the inactive protein kinases, which have been modified to contain the desired reactive groups (e.g. thiol groups, or thiol-containing residues) are based on the identification of one or more wild-type amino acid(s) on the surface of the target protein kinase that fall within that approximate 10-angstrom radius from the binding site of interest (which may have been first revealed as a result of the alteration resulting the stabilization of an inactive conformation). For the purposes of this measurement, any amino acid having at least one atom falling within the about 10 angstrom radius from any atom of an amino acid within the binding site of interest is a potential residue to be modified to a thiol containing residue.

Preferred residues for modification are those that are solvent-accessible. Solvent accessibility may be calculated from structural models using standard numeric (Lee, B. & Richards, F. M. J. Mol. Biol 55:379-400 (1971); Shrake, A. & Rupley, J. A. J. Mol. Biol. 79:351-371 (1973)) or analytical (Connolly, M. L. Science 221:709-713 (1983); Richmond, T. J. J. Mol. Biol. 178:63-89 (1984)) methods. For example, a potential cysteine variant is considered solvent-accessible if the combined surface area of the carbon-beta (CB), or sulfur-gamma (SG) is greater than 21 Å² when calculated by the method of Lee and Richards (Lee, B. & Richards, F. M. J. Mol. Biol 55:379-400 (1971)). This value represents approximately 33% of the theoretical surface area accessible to a cysteine side-chain as described by Creamer et al. (Creamer, T. P. et al. Biochemistry 34:16245-16250 (1995)).

It is also preferred that the residue to be mutated to cysteine, or another thiol-containing amino acid residue for tethering purposes, not participate in hydrogen-bonding with backbone atoms or, that at most, it interacts with the backbone through only one hydrogen bond. Wild-type residues where the side-chain participates in multiple (>1) hydrogen bonds with other side-chains are also less preferred. Variants for which all standard rotamers (chi1 angle of −60°, 60°, or 180°) can introduce unfavorable steric contacts with the N, CA, C, O, or CB atoms of any other residue are also less preferred. Unfavorable contacts are defined as interatomic distances that are less than 80% of the sum of the van der Waals radii of the participating atoms.

Additionally, residues found on convex “ridge” regions adjacent to concave surfaces are more preferred while those within concave regions are less preferred cysteine residues to be modified. Convexity and concavity can be calculated based on surface vectors (Duncan, B. S. & Olson, A. J. Biopolymers 33:219-229 (1993)) or by determining the accessibility of water probes placed along the molecular surface (Nicholls, A. et al. Proteins 11:281-296 (1991); Brady, G. P., Jr. & Stouten, P. F. J. Comput. Aided Mol. Des. 14:383-401 (2000)). Residues possessing a backbone conformation that is nominally forbidden for L-amino acids (Ramachandran, G. N. et al. J. Mol. Biol. 7:95-99 (1963); Ramachandran, G. N. & Sasisekharahn, V. Adv. Prot. Chem. 23:283-437 (1968)) are less preferred targets for modification to a cysteine. Forbidden conformations commonly feature a positive value of the phi angle.

Other preferred variants are those which, when mutated to cysteine and linked via a disulfide bond to an alkyl tether, would possess a conformation that directs the atoms of that tether towards the binding site of interest. Two general procedures can be used to identify these preferred variants. In the first procedure, a search is made of unique structures (Hobohm, U. et al. Protein Science 1:409-417 (1992)) in the Protein Databank (Berman, H. M. et al. Nucleic Acids Research 28:235-242 (2000)) to identify structural fragments containing a disulfide-bonded cysteine at position j in which the backbone atoms of residues j−1, j, and j+1 of the fragment can be superimposed on the backbone atoms of residues i−1, i, and i+1 of the target molecule with an RMSD of less than 0.75 A². If fragments are identified that place the CB atom of the residue disulfide-bonded to the cysteine at position j closer to any atom of the site of interest than the CB atom of residue i (when mutated to cysteine), position i is considered preferred. In an alternative procedure, the residue at position i is computationally “mutated” to a cysteine and capped with an S-Methyl group via a disulfide bond.

In still other embodiments, in addition to mutating a naturally occurring non-cysteine residue to a cysteine at a site of interest, one or more naturally occurring cysteines outside of the site of interest can be mutated to a non-cysteine residue (such as alanine or serine) to prevent unwanted labeling. In particular, those naturally occurring cysteines outside of the site of interest and are reactive to cystamine are candidates for being “scrubbed” (mutated to a non-cysteine residue).

Further details of identifying binding site(s) of interest for tethering purposes on the protein kinase targets of the invention are provided in PCT publication WO 03/014308 and co-pending application Ser. No. 10/214,419, filed on Aug. 5, 2002, which claims priority from provisional patent application Ser. No. 60/310,725, filed on Aug. 7, 2001, the entire disclosures of which are hereby expressly incorporated by reference.

Illustrative examples of kinase mutants where a non-native cysteine is introduced into at one or more sites of interest are described below.

For the AKT1 kinase, the following cysteine mutants are illustrative examples of mutants that are used for Tethering: L156C AKT1; K158C AKT1; T160C AKT1; F161C AKT1; K194C AKT1; E198C AKT1; M227C AKT1; E278C AKT1; T291C AKT1; K297C AKT1.

For the AKT2 kinase, the following cysteine mutants are illustrative examples of mutants that are used for Tethering: K158C AKT2; K160C AKT2; T162C AKT2; F163C AKT2; H196C AKT2; E200C AKT2; M229C AKT2; E279C AKT2; T292C AKT2; K298C AKT2.

For the AKT3 kinase, the following cysteine mutants are illustrative examples of mutants that are used for Tethering: L154C AKT3; K156C AKT3; T158C AKT3; F159C AKT3; H192C AKT3; E196C AKT3; M225C AKT3; E274C AKT3; T288C AKT3; K294C AKT3.

For the BLK kinase, the following cysteine mutants are illustrative examples of mutants that are used for Tethering: L246C BLK; S248C BLK; Q151C BLK; F251C BLK; A279C BLK; E283C BLK; T311C BLK; A363C BLK; A376C BLK; R382C BLK.

For the BTK kinase, the following cysteine mutants are illustrative examples of mutants that re used for Tethering: L408C BTK; T410C BTK; Q313C BTK; F413C BTK; E441C BTK; E445C BTK; T474C BTK; R525C BTK; S538C BTK; R544C BTK.

For the CDK1 kinase, the following cysteine mutants are illustrative examples of mutants that are used for Tethering: I10C CDK1; E12C CDK1; T14C CDK1; Y15C CDK1; S53C CDK1; E57C CDK1; F80C CDK1; Q432C CDK1; A145C CDK1; R151C CDK1.

For the CDK2 kinase, the following cysteine mutants are illustrative examples of mutants that are used for Tethering: I10C CDK2; E12C CDK2; T14C CDK2; Y15C CDK2; S53C CDK2; E57C CDK2; F80C CDK2; Q431C CDK2; A144C CDK2; R150C CDK2.

For the CDK3 kinase, the following cysteine mutants are illustrative examples of mutants that are used for Tethering: I10C CDK3; E12C CDK3; T14C CDK3; Y15C CDK3; S53C CDK3; E57C CDK3; F80C CDK3; Q431C CDK3; A144C CDK3; R150C CDK3.

For the CDK4 kinase, the following cysteine mutants are illustrative examples of mutants that are used for Tethering: I12C CDK4; V14C CDK4; A16C CDK4; Y17C CDK4; R55C CDK4; L59C CDK4; F93C CDK4; E153C CDK4; A157C CDK4; R163C CDK4.

For the CDK5 kinase, the following cysteine mutants are illustrative examples of mutants that are used for Tethering: I10C CDK5; E12C CDK5; T14C CDK5; Y15C CDK5; E57C CDK5; F80C CDK5; Q430C CDK5; A143C CDK5; R149C CDK5.

For the CDK6 kinase, the following cysteine mutants are illustrative examples of mutants that are used for Tethering: I19C CDK6; E21C CDK6; A23C CDK6; Y24C CDK6; A63C CDK6; H67C CDK6; F98C CDK6; Q449C CDK6; A162C CDK6; R168C CDK6.

For the CDK7 kinase, the following cysteine mutants are illustrative examples of mutants that are used for Tethering: L18C CDK7; E20C CDK7; Q22C CDK7; F23C CDK7; R61C CDK7; L65C CDK7; F91C CDK7; N141C CDK7; A154C CDK7; K161C CDK7.

For the CDK8 kinase, the following cysteine mutants are illustrative examples of mutants that are used for Tethering: V27C CDK8; R29C CDK8; T31C CDK8; Y32C CDK8; R65C CDK8; L69C CDK8; F97C CDK8; A1S5C CDK8; A172C CDK8; H178C CDK8.

For the CDK9 kinase, the following cysteine mutants are illustrative examples of mutants that are used for Tethering: I25C CDK9; Q27C CDK9; T29C CDK9; F30C CDK9; R65C CDK9; I69C CDK9; F103C CDK9; A153C CDK9; A166C CDK9; R172C CDK9.

For the CSK kinase, the following cysteine mutants are illustrative examples of mutants that are used for Tethering: I201C CSK; K203C CSK; E205C CSK; F206C CSK; A232C CSK; E236C CSK; T266C CSK; R318C CSK; S331C CSK; K337C CSK.

For the EGFR1 kinase, the following cysteine mutants are illustrative examples of mutants that are used for Tethering: L718C EGFR1; S720C EGFR1; A722C EGFR1; F723C EGFR1; E758C EGFR1; E762C EGFR1; T790C EGFR1; R841C EGFR1; T854C EGFR1; K860C EGFR1.

For the ERB2 (also referred to as ErbB2) kinase, the following cysteine mutants are illustrative examples of mutants that are used for Tethering: L726C ERB2; S728C ERB2; A730C ERB2; F731C ERB2; E766C ERB2; E770C ERB2; T798C ERB2; R849C ERB2; T862C ERB2; R868C ERB2.

For the ERB4 (also referred to as ErbB4) kinase, the following cysteine mutants are illustrative examples of mutants that are used for Tethering: L724C ERB4; S726C ERB4; A728C ERB4; F729C ERB4; E764C ERB4; E768C ERB4; T796C ERB4; R847C ERB4; T860C ERB4; R864C ERB4.

For the ERK1 kinase, the following cysteine mutants are illustrative examples of mutants that are used for Tethering: I48C ERK1; E50C ERK1; A52C ERK1; Y53C ERK1; R84C ERK1; E88C ERK1; Q122C ERK1; S170C ERK1; R189C ERK1.

For the ERK2 kinase, the following cysteine mutants are illustrative examples of mutants that are used for Tethering: I31C ERK2; E33C ERK2; A35C ERK2; Y36C ERK2; R67C ERK2; E71C ERK2; Q105C ERK2; S153C ERK2; R172C ERK2.

For the ERK3 kinase, the following cysteine mutants are illustrative examples of mutants that are used for Tethering: L26C ERK3; G30C ERK3; N31C ERK3; H61C ERK3; E65C ERK3; Q108C ERK3; A156C ERK3; G170C ERK3; R176C ERK3.

For the ERK4 kinase, the following cysteine mutants are illustrative examples of mutants that are used for Tethering: L26C ERK4; F28C ERK4; V30C ERK4; N31C ERK4; H61C ERK4; E65C ERK4; Q105C ERK4; A153C ERK4; G167C ERK4; R173C ERK4.

For the ERK5 kinase, the following cysteine mutants are illustrative examples of mutants that are used for Tethering: I60C ERK5; N62C ERK5; A64C ERK5; Y65C ERK5; R97C ERK5; E101C ERK5; L136C ERK5; S185C ERK5; G198C ERK5; R204C ERK5.

For the ERK6 kinase, the following cysteine mutants are illustrative examples of mutants that are used for Tethering: V33C ERK6; S35C ERK6; A37C ERK6; Y38C ERK6; R70C ERK6; E74C ERK6; M109C ERK6; G157C ERK6; L170C ERK6; R176C ERK6.

For the FAK1 kinase, the following cysteine mutants are illustrative examples of mutants that are used for Tethering: I428C FAK1; E430C FAK2; Q333C FAK1; F433C FAK1; K467C FAK1; E471C FAK1; M499C FAK1; R550C FAK1; G563C FAK1; R569C FAK1.

For the FGFR1 kinase, the following cysteine mutants are illustrative examples of mutants that are used for Tethering: L484C FGFR1; E486C FGFR1; F489C FGFR1; L528C FGFR1; M532C FGFR1; V561C FGFR1; R627C FGFR1; A640C FGFR1; R646C FGFR1.

For the FGFR2 kinase, the following cysteine mutants are illustrative examples of mutants that are used for Tethering: L487C FGFR2; E489C FGFR2; F492C FGFR2; L531C FGFR2; M535C FGFR2; V564C FGFR2; R630C FGFR2; A643C FGFR2; R649C FGFR2.

For the FGFR3 kinase, the following cysteine mutants are illustrative examples of mutants that are used for Tethering: L478C FGFR3; E480C FGFR3; F483C FGFR3; L522C FGFR3; M526C FGFR3; V555C FGFR3; R621C FGFR3; A634C FGFR3; R640C FGFR3.

For the FGFR4 kinase, the following cysteine mutants are illustrative examples of mutants that are used for Tethering: L473C FGFR4; E475C FGFR4; F478C FGFR4; L517C FGFR4; M521C FGFR4; V550C FGFR4; R616C FGFR4; A629C FGFR4; R635C FGFR4.

For the FYN kinase, the following cysteine mutants are illustrative examples of mutants that are used for Tethering: L276C FYN; N278C FYN; Q181C FYN; F281C FYN; S309C FYN; E313C FYN; T341C FYN; A393C FYN; A406C FYN; R412C FYN.

For the HCK kinase, the following cysteine mutants are illustrative examples of mutants that are used for Tethering: L268C HCK; A270C HCK; Q173C HCK; F273C HCK; A301C HCK; E305C HCK; T333C HCK; A385C HCK; A398C HCK; R404C HCK.

For the IKK-a kinase, the following cysteine mutants are illustrative examples of mutants that are used for Tethering: L21C IKK-a; T23C IKK-a; G25C IKK-a; F26C IKK-a; R57C IKK-a; E61C IKK-a; M95C IKK-a; E148C IKK-a; 1164C IKK-a; K170C IKK-a.

For the IKK-b kinase, the following cysteine mutants are illustrative examples of mutants that are used for Tethering: L21C IKK-b; T23C IKK-b; G25C IKK-b; F26C IKK-b; R57C IKK-b; E61C IKK-b; M96C IKK-b; E149C IKK-b; 1165C IKK-b; K171C IKK-b.

For the IKK-e kinase, the following cysteine mutants are illustrative examples of mutants that are used for Tethering: L15C IKK-e; Q17C IKK-e; A19C IKK-e; T20C IKK-e; V51C IKK-e; E55C IKK-e; M86C IKK-e; G139C IKK-e; T156C IKK-e; R163C IKK-e.

For the JAK1 kinase, the following cysteine mutants are illustrative examples of mutants that are used for Tethering: L870C JAK1; E872C JAK1; H874C JAK1; F875C JAK1; D909C JAK1; E913C JAK1; M944C JAK1; R995C JAK1; G1008C JAK1; K1014C JAK1.

For the JAK2 kinase, the following cysteine mutants are illustrative examples of mutants that are used for Tethering: L855C JAK2; L857C JAK2; N859C JAK2; F860C JAK2; D894C JAK2; E898C JAK2; M929C JAK2; R980C JAK2; G993C JAK2; K999C JAK2.

For the JAK3 kinase, the following cysteine mutants are illustrative examples of mutants that are used for Tethering: L828C JAK3; K830C JAK3; N832C JAK3; F833C JAK3; D867C JAK3; E871C JAK3; M902C JAK3; R953C JAK3; A966C JAK3; K972C JAK3.

For the JNK1 kinase, the following cysteine mutants are illustrative examples of mutants that are used for Tethering: I32C JNK1; S34C JNK1; A36C JNK1; Q37C JNK1; R69C JNK1; E73C JNK1; M108C JNK1; S155C JNK1; L168C JNK1; R174C JNK1.

For the JNK2 kinase, the following cysteine mutants are illustrative examples of mutants that are used for Tethering: I32C JNK2; S34C JNK2; A36C JNK2; Q37C JNK2; R69C JNK2; E73C JNK2; M108C JNK2; S155C JNK2; L168C JNK2; R174C JNK2.

For the JNK3 kinase, the following cysteine mutants are illustrative examples of mutants that are used for Tethering: I70C JNK3; S72C JNK3; A74C JNK3; Q75C JNK3; R107C JNK3; E111C JNK3; M146C JNK3; S193C JNK3; L206C JNK3; R212C JNK3.

For the Lck kinase, the following cysteine mutants are illustrative examples of mutants that are used for Tethering: L250C Lck; A252C Lck; Q155C Lck; F255C Lck; A283C Lck; E287C Lck; T315C Lck; A367C Lck; A380C Lck; R386C Lck.

For the LYN kinase, the following cysteine mutants are illustrative examples of mutants that are used for Tethering: L252C LYN; A254C LYN; Q157C LYN; F257C LYN; A285C LYN; E289C LYN; T318C LYN; A370C LYN; A383C LYN; D389C LYN.

For the MAPK kinase, the following cysteine mutants are illustrative examples of mutants that are used for Tethering: V30C MAPK; S32C MAPK; A34C MAPK; Y35C MAPK; R67C MAPK; E71C MAPK; T106C MAPK; S154C MAPK; L167C MAPK; R173C MAPK.

For the NIK kinase, the following cysteine mutants are illustrative examples of mutants that are used for Tethering: L406C NIK; R408C NIK; S410C NIK; F411C NIK; F436C NIK; E439C NIK; M469C NIK; D519C NIK; V540C NIK.

For the PAK1 kinase, the following cysteine mutants are illustrative examples of mutants that are used for tethering: I276C PAK1; Q179C PAK1; A280C PAK1; S281C PAK1; N314C PAK1; V318C PAK1; M344C PAK1; D393C PAK1; T406C PAK1; A412C PAK1.

For the PAK2 kinase, the following cysteine mutants are illustrative examples of mutants that are used for Tethering: I255C PAK2; Q158C PAK2; A259C PAK2; S260C PAK2; N293C PAK2; V297C PAK2; M323C PAK2; D372C PAK2; T385C PAK2; A391C PAK2.

For the PAK3 kinase, the following cysteine mutants are illustrative examples of mutants that are used for Tethering: I274C PAK3; Q177C PAK3; A278C PAK3; S279C PAK3; N312C PAK3; V316C PAK3; M342C PAK3; D391C PAK3; T404C PAK3; A410C PAK3.

For the PAK4 kinase, the following cysteine mutants are illustrative examples of mutants that are used for Tethering: I327C PAK4; E329C PAK4; S331C PAK4; R332C PAK4; N365C PAK4; I369C PAK4; M395C PAK4; D444C PAK4; S457C PAK4; A463C PAK4.

For the PAK5 kinase, the following cysteine mutants are illustrative examples of mutants that are used for Tethering: I455C PAK5; E457C PAK5; S459C PAK5; T460C PAK5; N492C PAK5; I496C PAK5; M523C PAK5; D572C PAK5; D585C PAK5; A591C PAK5.

For the PDGFR-a kinase, the following cysteine mutants are illustrative examples of mutants that are used for Tethering: L599C PDGFR-a; S601C PDGFR-a; A603C PDGFR-a; F604C PDGFR-a; L641C PDGFR-a; L645C PDGFR-a; T674C PDGR-a; R822C PDGFR-a; R841C PDGFR-a.

For the PDGFR-b kinase, the following cysteine mutants are illustrative examples of mutants that are used for Tethering: L606C PDGFR-b; S608C PDGFR-b; A700C PDGFR-b; F701C PDGFR-b; L648C PDGFR-b; L652C PDGFR-b; T681C PDGFR-b; R830C PDGFR-b; R849C PDGFR-b.

For the PIM1 kinase, the following cysteine mutants are illustrative examples of mutants that are used for Tethering: L44C PIM1; S46C PIM1; G48C PIM1; F49C PIM1; M87C PIM1; L91C PIM1; E121C PIM1; E171C PIM1; E171C PIM1; I185C PIM1; A192C PIM1.

For the A-Raf kinase, the following cysteine mutants are illustrative examples of mutants that are used for tethering: I316C A-Raf; T318C A-Raf; S320C A-Raf; F321C A-Raf; A350C A-Raf; E354C A-Raf; T382C A-Raf; N433C A-Raf; G446C A-Raf; T452C A-Raf.

For the B-Raf kinase, the following cysteine mutants are illustrative examples of mutants that are used for Tethering: I462C B-Raf; S464C B-Raf; S466C B-Raf; F467C B-Raf; A496C B-Raf; E500C B-Raf; T528C B-Raf; N579C B-Raf; G592C B-Raf; T598C B-Raf.

For the C-Raf kinase, the following cysteine mutants are illustrative examples of mutants that are used for Tethering: I355C C-Raf; S357C C-Raf; S359C C-Raf; F-360C C-Raf; A389C C-Raf; E393C C-Raf; T421C C-Raf; N472C C-Raf; G485C C-Raf; T491C C-Raf.

For the SRC kinase, the following cysteine mutants are illustrative examples of mutants that are used for Tethering: L275C SRC; Q178C SRC; F280C SRC; A308C SRC; E402C SRC; T340C SRC; A392C SRC; A405C SRC; R411C SRC.

For the SRC2 kinase, the following cysteine mutants are illustrative examples of mutants that are used for Tethering: L269C SRC2; T271C SRC2; F274C SRC2; A302C SRC2; E306C SRC2; T334C SRC2; A386C SRC2; A399C SRC2; R405C SRC2.

For the STK1 kinase, the following cysteine mutants are illustrative examples of mutants that are used for Tethering: L616C STK1; S618C STK1; A620C STK1; F621C STK1; L658C STK1; L662C STK1; F691C STK1; R815C STK1, R834C STK1.

For the SYK kinase, the following cysteine mutants are illustrative examples of mutants that are used for Tethering: L377C SYK; S379C SYK; N381C SYK; F382C SYK; E416C SYK; E420C SYK; M448C SYK; R498C SYK; S511C SYK; K518C SYK.

For the TEC kinase, the following cysteine mutants are illustrative examples of mutants that are used for Tethering: L376C TEC; S378C TEC; L380C TEC; F381C TEC; D409C TEC; E413C TEC; T442C TEC; R493C TEC; S506C TEC; R513C TEC.

For the TGFR1 kinase, the following cysteine mutants are illustrative examples of mutants that are used for Tethering: I211C TGFR1; K213C TGFR1; R215C TGFR1; F216C TGFR1; F243C TGFR1; E247C TGFR1; S280C TGFR1; K337C TGFR1; A350C TGFR1; V357C TGFR1.

For the TGFR2 kinase, the following cysteine mutants are illustrative examples of mutants that are used for Tethering: V250C TGFR2; K252C TGFR2; R254C TGFR2; F255C TGFR2; K288C TGFR2; D292C TGFR2; T325C TGFR2; S383C TGFR2; L403C TGFR2.

For the TIE1 kinase, the following cysteine mutants are illustrative examples of mutants that are used for Tethering: I845C TIE1; E847C TIE1; N849C TIE1; F850C TIEL; F884C TIE1; L888C TIE1; I917C TIE1; R983C TIE1; A996C TIE 1; R1002C TIE.

For the TIE2 kinase, the following cysteine mutants are illustrative examples of mutants that are used for Tethering: I830C TIE2; E832C TIE2; N834C TIE2; F835C TIE2; F869C TIE2; L873C TIE2; I902C TIE2; R968C TIE2; A981C TIE2; R987C TIE2.

For the VEGFR1 kinase, the following cysteine mutants are illustrative examples of mutants that are used for Tethering: L834C VEGFR1; R836C VEGFR1; A838C VEGFR1; F839C VEGFR1; L876C VEGFR1; L880C VEGFR1; V910C VEGFR1; R1026C VEGFR1; R1045C VEGFR1.

For the VEGFR2 kinase, the following cysteine mutants are illustrative examples of mutants that are used for Tethering: L840C VEGFR2; R842C VEGFR2; A844C VEGFR2; F845C VEGFR2; L882C VEGFR2; L886C VEGFR2; V916C VEGFR2; R1032C VEGFR2; R1051C VEGFR2.

For the VEGFR3 kinase, the following cysteine mutants are illustrative examples of mutants that are used for Tethering: L851C VEGFR3; Y853C VEGFR3; A855C VEGFR3; F856C VEGFR3; L893C VEGFR3; L987C VEGFR3; V927C VEGFR3; R1041C VEGFR3; R1060C VEGFR3.

For the YES kinase, the following cysteine mutants are illustrative examples of mutants that are used for Tethering: L283C YES; Q286C YES; C287C YES; F288C YES; A316C YES; E320C YES; T348C YES; A400C YES; A413C YES; R419C YES.

For the ZAP-70 kinase, the following cysteine mutants are illustrative examples of mutants that are used for Tethering: L344C ZAP-70; N348C ZAP-70; F349C ZAP-70; E382C ZAP-70; E386C ZAP-70; M414C ZAP-70; R465C ZAP-70; S478C ZAP-70; and K485C ZAP-70.

Although this approach is typically exemplified with reference to a protein kinase target having a thiol functionality to screen a disulfide-containing library, other chemistries are also available and are readily used.

c. Tethering with Extenders

Tethering with extenders is a variation of Tethering described above that uses a Small Molecule Extender (SME) to form a target kinase-SME covalent complex. The SME has a first reactive functionality that is capable of forming a reversible or irreversible covalent bond with the target kinase and a second reactive functionality that is capable of forming a reversible or irreversible covalent bond with a ligand candidate. Thus, the SME forms a first covalent bond with the target kinase thereby forming a target kinase-SME covalent complex. In certain embodiments, the SME also includes a binding element that has an affinity for the SME binding site or a first site of interest. The second reactive functionality on the SME on the target kinase-SME covalent complex is used in Tethering to identify ligands that have an affinity for a site on the kinase that is adjacent to the SME binding site. This adjacent site is referred to as the second site of interest.

In certain embodiments, the first reactive functionality on a SME forms a irreversible covalent bond through the nucleophile or electrophile, preferably nucleophile, on the protein kinase target, thereby forming an irreversible protein kinase-SME complex. Preferred nucleophiles on the target protein kinase suitable for forming an irreversible kinase-SME complex include —SH, —OH, —NH₂ and —COOH usually arising from side chains of Cys, Ser or Thr, Lys and Asp or Glu respectively. Protein kinases may be modified (e.g. mutants or derivatives) to contain these nucleophiles or may contain them naturally. For example, BLK, BTK, EGFR1, ERB2, ERB4, ERK1, ERK2, FGFR1, FGFR2, FGFR3, FGFR4, etc. are examples of kinases containing suitable naturally occurring cysteine thiol nucleophiles.

In other embodiments, the second reactive functionality is a group capable of forming a disulfide bond. Illustrative examples of such a group include a free thiol (—SH), protected thiol (—SR′ where R′ is a thiol protecting group), and a disulfide (—SSR″ where R″ is a substituted or unsubstituted aliphatic or substituted or unsubstituted aryl).

The SME may, but does not have to, include a portion that has binding affinity (i.e. is capable of bonding to) a first site of interest on the target kinase. Even if the SME does not include such portion, it must be of appropriate length and flexibility to ensure that the ligand candidates have free access to the second site of interest on the target.

FIG. 1B is a schematic illustration of one embodiment of Tethering with extenders. As shown, a target that includes a thiol is contacted with an extender comprising a first functionality -LG that is capable of forming a covalent bond with the reactive thiol and a second functionality second functionality -SPG that is capable of forming a disulfide bond. The extender binds to the first site of interest and forms a target-extender covalent complex which is then contacted with a plurality of ligand candidates to identify a ligand for a second site of interest. The extender provides one binding determinant (circle) and the ligand candidate provides the second binding determinant (square) and the resulting binding determinants are linked together to form a conjugate compound.

As illustrated in FIG. 1B, in certain embodiments, the SME includes a binding element that has affinity for the SME binding site. Thus, compounds having known affinity for kinases can be modified to be SME's by adding the first reactive functionality (-LG in FIG. 1B) and the second reactive functionality (-SPG in FIG. 1B).

Suitable first reactive functionalities include groups that are capable of undergoing SN2-like or Michael-type addition and thus forming an irreversible covalent bond with the target kinase. Examples of SME's having such groups are further described below. For the purposes of illustration, the SME's are shown schematically where □ optionally includes a binding element for the intended SME binding site and —SR′ is the second reactive functionality that is capable of forming a disulfide bond.

α-halo acids: F, Cl and Br substituted a to a COOH, PO₃H₂ or P(OR)O₂H acid that is part of the SME can form a thioether with the thiol of the target kinase. Illustrative examples of generic α-halo acids are shown below. 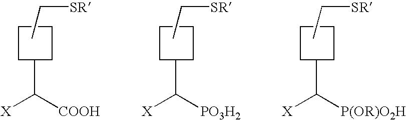
where X is the halogen, R is C1-C20 unsubstituted aliphatic, C1-C20 substituted aliphatic, unsubstituted aryl or substituted aryl, and R′ is H, SCH₃, S(CH₂)ₙA, where A is OH, COOH, SO₃H, CONH₂ or NH₂ and n is 1 to 5, preferably n is 2 to 4.

Fluorophosph(on)ates: These are Sarin-like compounds which react readily with both SH and OH nucleophiles. Illustrative examples of general fluorophosph(on)ates are shown below. 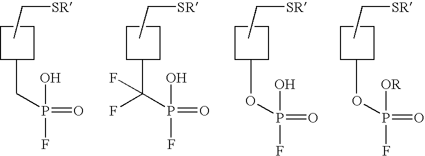
where R and R′ are as defined above.

Epoxides, aziridines and thiiranes: SME's containing these reactive functional groups are capable of undergoing SN2 ring opening reactions with —SH, —OH and —COOH nucleophiles. Preferred examples of the latter are aspartyl proteases like β-secretase (BASE). Preferred generic examples of epoxides, aziridines and thiiranes are shown below. 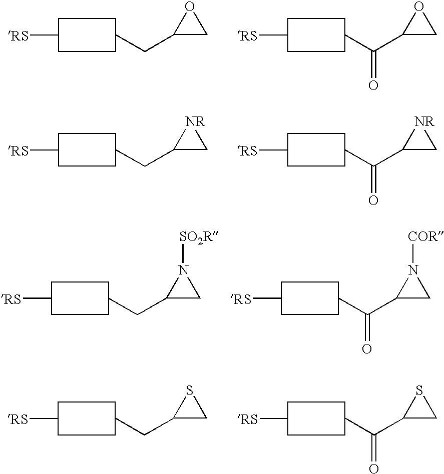
Here, R′ is as defined above, R is usually H or lower alkyl and R″ is lower alkyl, lower alkoxy, OH, NH₂ or SR′. In the case of thiiranes the group SR′ is optionally present because upon nucleophilic attack and ring opening a free thiol is produced which may be used in the subsequent extended tethering reaction.

Halo-methyl ketones/amides: These compounds have the form —(C═O)—CH₂—X. Where X may be a large number of good leaving groups like halogens, N₂, O—R (where R may be substituted or unsubstituted heteroaryl, aryl, alkyl, —(P═O)Ar₂, —N—O—(C═O) aryl/alkyl, —(C═O) aryl/alkyl/alkylaryl and the like), S-Aryl, S-heteroaryl and vinyl sulfones.

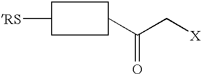

Fluromethylketones are simple examples of this class of activated ketones which result in the formation of a thioether when reacted with a thiol containing protein. Other well known examples include acyloxymethyl ketones like benzoyloxymethyl ketone, aminomethyl ketones like phenylmethylaminomethyl ketone and sulfonylaminomethyl ketones. These and other types of suitable compounds are reviewed in J. Med. Chem. 43(18) p 3351-71, Sep. 7, 2000.

Electrophilic aromatic systems: Examples of these include 7-halo-2,1,3-benzoxadiazoles and ortho/para nitro substituted halobenzenes. 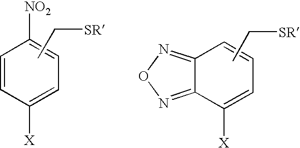
Compounds of this type form arylalkylthioethers with protein kinases containing a thiol.

Other suitable SN2 like reactions suitable for formation of covalent bonds with protein kinase nucleophiles include formation of a Schiff base between an aldehyde and the amine group of lysine of enzymes like DNA repair proteins followed by reduction with for example NaCNBH₄.

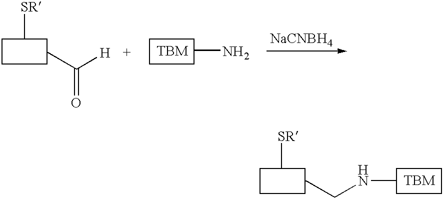

Michael-type additions: Compounds of the form —RC═CR-Q, or —C≡C-Q where Q is C(═O)H, C(═O)R, COOR, C(═O)NH₂, C(═O)NHR, CN, NO₂, SOR, SO₂R, where each R is independently substituted or unsubstituted alkyl, aryl, hydrogen, halogen or another Q can form Michael adducts with SR (where R is H, glutathione or S-lower alkyl substituted with NH₂ or OH), OH and NH₂ on the target protein kinase.

Boronic acids: These compounds can be used where the reactive nucleophile on the target kinase is a hydroxyl. For example serine, theonine, or tyrosine on a target kinase can be labeled to form kinase-SME complexes for use in the present invention. The formation of such a kinase-SME complex is shown below. 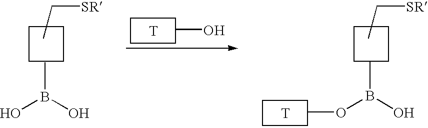
where R′ is as defined above.

In other embodiments, the first site of interest is the ATP binding pocket. In these embodiments, known compounds that target the ATP binding pocket of kinases can be modified to be an SME by adding first and second reactive functionalities. Illustrative examples of such SME's include those that contain purine or purine mimetics such as the following: 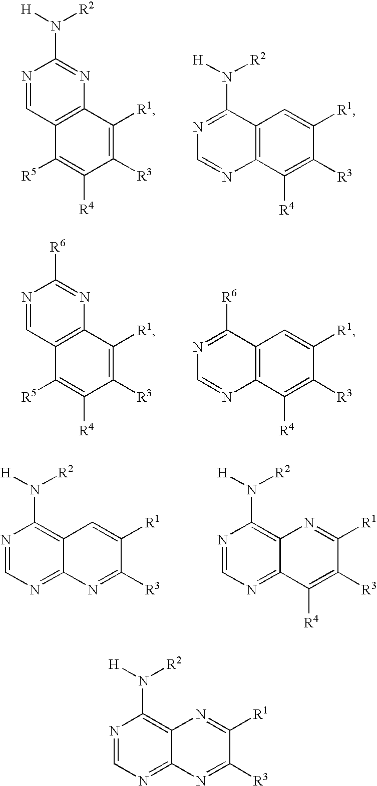
where R¹, R¹, R³, R⁴, R⁵, and R⁶ are each independently selected from the group consisting of hydrogen, C₁-C₅ alkyl, C₁-C₅ alkylamine, and aryl provided that at least one R group on the SME is a Michael acceptor or —(C═O)CH₂X where X is a halogen, and another R group is selected from —(CH₂)ₙ—SR′; —C(═O)—(CH₂)ₙ—SR′; —O—(CH₂)ₙ—SR′; —(CH₂)ₙ—SR′; and a thiol protecting group where R′ is hydrogen or sulfide and n is 1-5, preferably 2-4. In certain embodiments, R′ is —S(CH₂)ₙNH₂, —S(CH₂)ₙOH or —S(CH₂)ₙCOOH where n is 1-5, preferably 2-4. Illustrative examples of Michael acceptors include

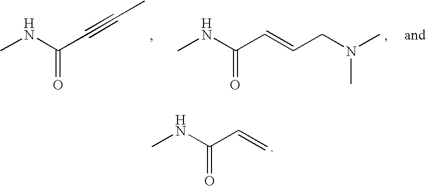

Illustrative examples of suitable SME's containg quinazolines include: 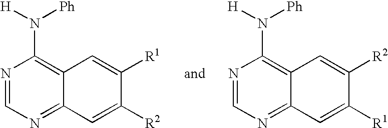
where R¹ is —NHC(═O)CH₂Cl, —NHC(═O)CH═CH₂ or —NHC(═O)CCH and R² is —(CH₂)ₘSSCH₂CH₂NH₂ where m is 1-3.

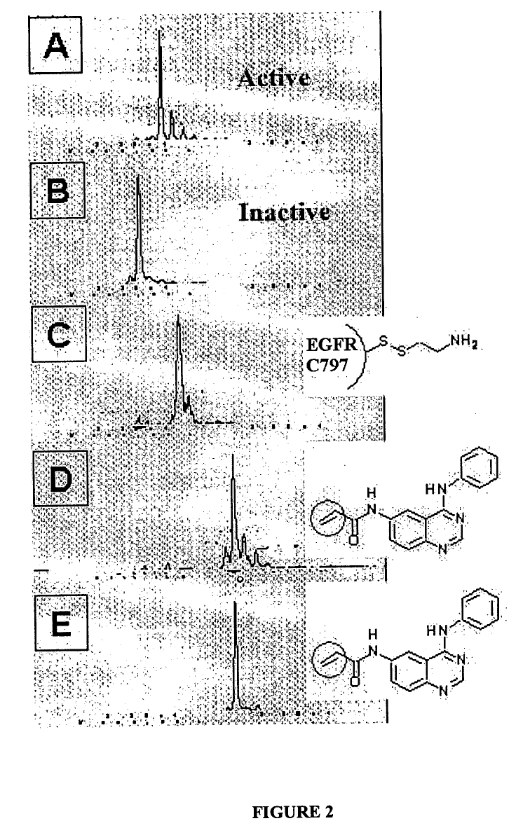

FIG. 2 illustrates the use of a quinazoline extender to form a kinase-extender covalent complex. FIG. 2A is the mass spectrometer profile of purified EGFR1 kinase domain in the active conformation. FIG. 2B is purified EGFR1 kinase domain in the inactive conformation. FIGS. 2C-E) are purified EGFR1 in the inactive conformation following incubation with C) cystamine, D) the quinazoline extender shown, and E) the quinazoline extender and cystamine.

Alternatively, Tethering can be used to identify novel ligands that bind to the ATP-binding pocket. For example, Tethering off the naturally occurring cysteine at the bottom of the ATP binding pocket in EGFR1 (C797), identified the expected purine and purine mimetic containing ligands along with several novel scaffolds. Representative ligand candidates with novel scaffolds include: 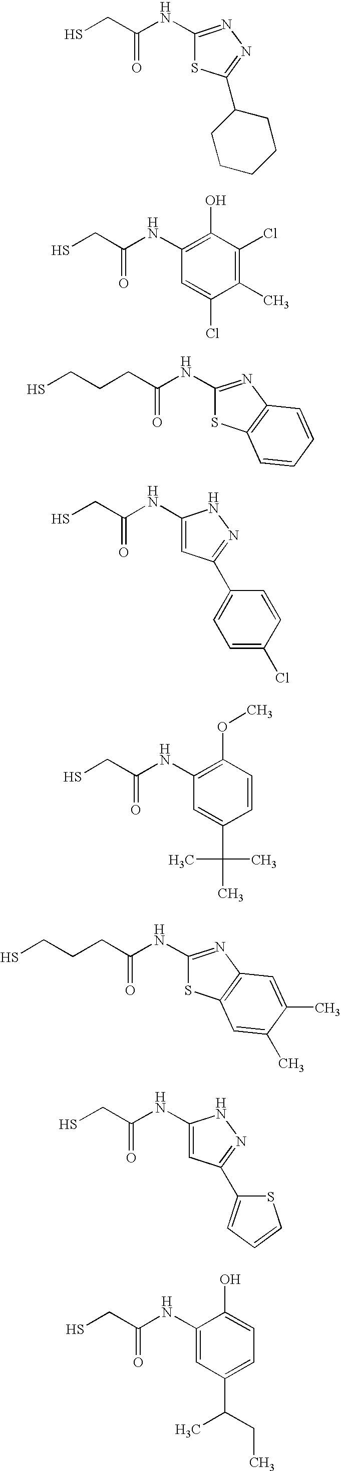 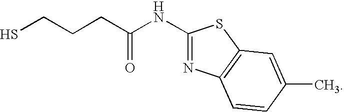
Illustrative examples of SME's that can be made using such scaffolds by adding first and second functionalities include but are not limited to:

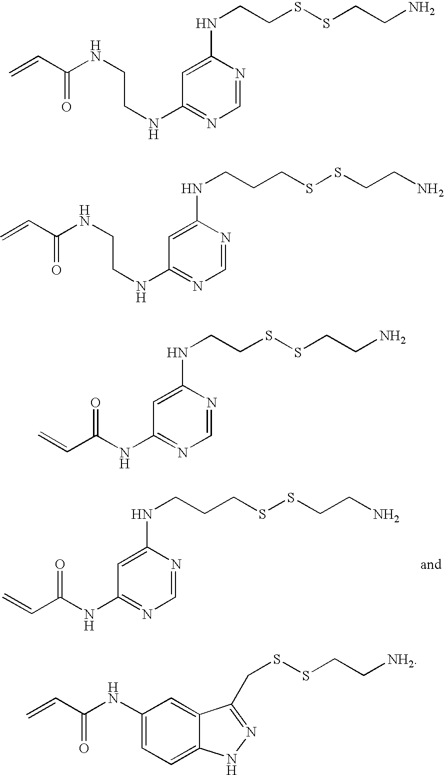

As described above, certain kinases already possess a naturally occurring cysteine within the ATP binding pocket that can be used to identify ligands that bind to this site. In addition to C797 of EGFR1, other examples of kinases that include a naturally occurring cysteine within the ATP binding pocket include: BLK (C318); BTK (C481); ERB2 (C805); ERB4 (C803); JAK3 (C909); TEC (C449). In addition to being used to identify novel ligands that bind to the ATP-binding pocket, these cysteines are also good candidates for Tethering with extenders. If SME's containing purine or purine mimetics are used (so that the SME binds to the ATP binding site), the resulting kinase-SME complex can be used to identify ligands to the adaptive binding region adjacent to the ATP binding site.

For kinases that do not have a naturally occurring cysteine at the ATP binding pocket, the following are illustrative examples of mutants where a cysteine is introduced at the appropriate location: E234C AKT1; E236C ALT2; E232C AKT3; D86C CDK1; D86C CDK2; D86C CDK3; D99C CDK4; D86C CDK5; D104C CDK6; D97C CDK7; D103C CDK8; D108C CDK9; S273C CSK; D128C ERK1; D111C ERK2; D114C ERK3; D111C ERK4; D142C ERK5; D115C ERK6; E506C FAK1; N568C FGFR1; N571C FGFR2; N562C FGFR3; N557C FGFR4; D348C FYN; S340C HCK; D102C IKK-a; D103C IKK-b; S93C IKK-e; S951C JAK1; S936C JAK2; N114C JNK1; N114C JNK2; N152C JNK3; S322C LCK; S325C LYN; D112C MAPK; S150C MEK1; S476C NIK; S351C PAK1; S330C PAK2; S349C PAK3; A402C PAK4; A530C PAK5; D861C PDGFR-a; D688C PDGFR-b; D128C PIM1; S389C A-Raf; S535C B-Raf; S428C C-Raf; S347C SRC; S341C SRC2; D698C STK1; P455C SYK; S287C TGFR1; N332C TGFR2; N924C TE1; N909C TIE2; N917C VEGFR1; N923C VEGFR2; N934C VEGFR3; S355C YES; P421C ZAP-70.

Although Tethering with extenders has been primarily described with target kinases having reactive thiols and extenders having a group capable of forming a covalent bond with the thiol, other chemistries can be used. For example, the amino group of lysines are alternative nucleophiles on the target kinases. The following extender is an exemplary extender that is capable of forming a covalent bond with a lysine 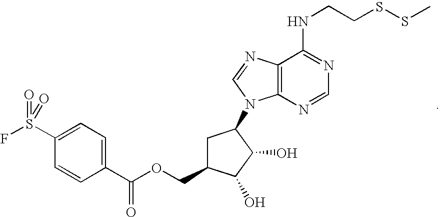
Because the ATP binding pocket includes a conserved lysine, the 5′-(p-fluorosulfonylbenzoyl)adenosine-based extender can be used with any kinase without the need for making a cysteine mutation in this site. The precursor for installing a masked thiol onto the adenosine-containing compound is made by reacting reacting commercially available N-Boc-cysteamine with commercially available methanethiosulfonic acid S-methyl ester, followed by deprotection of the Boc group to generate the hydrochloride salt. The resulting compound is reacted with commercially available 6-chloroadenosine. The installation of the electrophile as described in J. Biol. Chem. 250: 8140-8147 (1975) and Biochemistry 16: 1333-1342 (1977). Once this extender is reacted with the lysine in the ATP-binding site, the masked thiol can be reduced to the free thiol by a reducing agent such as 2-mercaptoethanol. The resulting kinase-extender complex can then be used in Tethering as described above.

While it is usually preferred that the attachment of the SME does not denature the target, the kinase-SME complex may also be formed under denaturing conditions, followed by refolding the complex by methods known in the art. Moreover, the SME and the covalent bond should not substantially alter the three-dimensional structure of the target protein kinase, so that the ligands will recognize and bind to a binding site of interest on the target with useful site specificity. Finally, the SME should be substantially unreactive with other sites on the target under the reaction and assay conditions.

d. Library of Sulfhydryl-Containing Fragments

The assembly of a collection of drug-like fragments or “monophores” that display a masked sulfhydryl group is used in certain embodiments of Tethering. The sulfhydryl is installed such that the fragment can participate in a disulfide exchange reaction with the cysteine residue on a kinase target. The monophores fragments are also broadly representative of recognized and unique drug-like pharmacophores and fragments thereof. At a minimum, candidate fragments satisfy two primary criteria. First, they contain a functional group that will permit the installation of a disulfide linker. Suitable functional groups include a free amine, carboxylate, sulfonyl chloride, isocyanate, aldehyde, ketone, etc. Second, they are chosen such that the combination of two such entities results in a product with drug-like physical properties, including molecular weight (approximately 500 Da or less) and hydrophobicity (cLog P between −1 and 5).

Chemistries for making the sulfhydryl-containing fragments as well as practicing the other aspect of the present invention such as forming a reversible or irreversible covalent bond between reactive groups on a protein kinase, making SME's, and compound advancement, are well known in the art, and are described in basic textbooks, such as, e.g. March, Advanced Organic Chemistry, John Wiley & Sons, New York, 4^{th} edition, 1992. Reductive aminations between aldehydes and ketones and amines are described, for example, in March et al., supra, at pp. 898-900; alternative methods for preparing amines at page 1276; reactions between aldehydes and ketones and hydrazide derivatives to give hydrazones and hydrazone derivatives such as semicarbazones at pp. 904-906; amide bond formation at p. 1275; formation of ureas at p. 1299; formation of thiocarbamates at p. 892; formation of carbamates at p. 1280; formation of sulfonamides at p. 1296; formation of thioethers at p. 1297; formation of disulfides at p. 1284; formation of ethers at p. 1285; formation of esters at p. 1281; additions to epoxides at p. 368; additions to aziridines at p. 368; formation of acetals and ketals at p. 1269; formation of carbonates at p. 392; formation of enamines at p. 1264; metathesis of alkenes at pp. 1146-1148 (see also Grubbs et al., Acc. Chem. Res. 28:446-453 [1995]); transition metal-catalyzed couplings of aryl halides and sulfonates with alkanes and acetylenes, e.g. Heck reactions, at pp. 717-178; the reaction of aryl halides and sulfonates with organometallic reagents, such as organoboron, reagents, at p. 662 (see also Miyaura et al., Chem. Rev. 95:2457 [1995]); organotin, and organozinc reagents, formation of oxazolidines (Ede et al., Tetrahedron Letts. 28:7119-7122 [1997]); formation of thiazolidines (Patek et al., Tetrahedron Letts. 36:2227-2230 [1995]); amines linked through amidine groups by coupling amines through imidoesters (Davies et al., Canadian J. Biochem. 50:416-422 [1972]), and the like. In particular, disulfide-containing small molecule libraries may be made from commercially available carboxylic acids and protected cysteamine (e.g. mono-BOC-cysteamine) by adapting the method of Parlow et al., Mol. Diversity 1:266-269 (1995), and can be screened for binding to polypeptides that contain, or have been modified to contain, reactive cysteines. All of the references cited in this section are hereby expressly incorporated by reference.

The monophores library can be derived from commercially available compounds that satisfy the above criteria. However, many motifs common in biologically active compounds are rare or absent in commercial sources of chemicals. Therefore, the fragment collection is preferably supplemented by synthesizing monophores fragments that help fill these gaps. A typical library can contain 10,000 or more compounds.

e. Detection and Identification of Ligands Bound to a Target

The ligands bound to a target (or to a target-SME complex) can be readily detected and identified by mass spectroscopy (MS). MS detects molecules based on mass-to-charge ratio (m/z) and thus can resolve molecules based on their sizes (reviewed in Yates, Trends Genet. 16: 5-8 [2000]). A mass spectrometer first converts molecules into gas-phase ions, then individual ions are separated on the basis of m/z ratios and are finally detected. A mass analyzer, which is an integral part of a mass spectrometer, uses a physical property (e.g. electric or magnetic fields, or time-of-flight [TOF]) to separate ions of a particular m/z value that then strikes the ion detector. Mass spectrometers are capable of generating data quickly and thus have a great potential for high-throughput analysis. MS offers a very versatile tool that can be used for drug discovery. Mass spectroscopy may be employed either alone or in combination with other means for detection or identifying the organic compound ligand bound to the target. Techniques employing mass spectroscopy are well known in the art and have been employed for a variety of applications (see, e.g., Fitzgerald and Siuzdak, Chemistry & Biology 3: 707-715 [1996]; Chu et al., J. Am. Chem. Soc. 118: 7827-7835 [1996]; Siudzak, Proc. Natl. Acad. Sci. USA 91: 11290-11297 [1994]; Burlingame et al., Anal. Chem. 68: 599R-651R[1996]; Wu et al., Chemistry & Biology 4: 653-657 [1997]; and Loo et al., Am. Reports Med. Chem. 31: 319-325 [1996]).

However, the scope of the instant invention is not limited to the use of MS. In fact, any other suitable technique for the detection of the adduct formed between the protein kinase target molecule and the library member can be used. For example, one may employ various chromatographic techniques such as liquid chromatography, thin layer chromatography and likes for separation of the components of the reaction mixture so as to enhance the ability to identify the covalently bound organic molecule. Such chromatographic techniques may be employed in combination with mass spectroscopy or separate from mass spectroscopy. One may optionally couple a labeled probe (fluorescently, radioactively, or otherwise) to the liberated organic compound so as to facilitate its identification using any of the above techniques. In yet another embodiment, the formation of the new bonds liberates a labeled probe, which can then be monitored. Other techniques that may find use for identifying the organic compound bound to the target molecule include, for example, nuclear magnetic resonance (NMR), capillary electrophoresis, X-ray crystallography, and the like, all of which will be well known to those skilled in the art.

f. Identification of Kinase Inhibitors from Tethering

As described above, in certain embodiments, pools containing compounds that covalently modify the kinase or the kinase-extender covalent complex are identified by mass spectrometry (MS) analysis. From the deconvoluted MS profile, the molecular weight of the bound compound can be precisely calculated, and thus its identity in the pool determined. The discrete compound is then tested alone to determine if it can covalently modify the kinase or the kinase-extender complex. Each screen is likely to identify multiple hits. Hits are prioritized according to their relative binding affinities and according to their relative preference for the inactive enzyme conformation. Relative enzyme binding affinities, expressed as a BME₅₀, are then determined using a BME titration curve to determine the concentration that allows 50% modification while using a constant concentration of compound. From this one can easily rank the compounds, based upon their binding affinities.

Upon compilation of the confirmed monophores hits, additional valuable information can be gained from analyzing the structure-activity relationship (SAR) between hit compounds and their relatives in the monophores library. For example, if several hit molecules for a particular kinase or kinase-extender pair fall into a closely related family, one may then go back to the monophores library and find structurally similar compounds that were not selected in the initial screen. These relatives are re-screened as discrete compounds to verify their activity (or inactivity), followed by rank ordering of the entire family in terms of affinity for both the active and inactive enzyme conformations. From this dataset, one can identify features critical to activity, and potential sites of modification the alteration of which is expected to improve affinity.

In parallel with the SAR studies, the covalently attached compounds or extender-compounds are co-crystallized with the target kinase domain. Alternatively, the compounds or the extender-compound complexes lacking the Michael acceptor are synthesized and either soaked into crystals of the relevant kinase or co-crystallized with the relevant kinase. X-ray data are collected and programmed by using commercially available equipments and softwares.

The identified ligands can be advanced into lead compounds by any number of methods known in the art. In certain embodiments, compound libraries are made based upon the identified fragments. In other embodiments, traditional medicinal chemistry approaches are used.

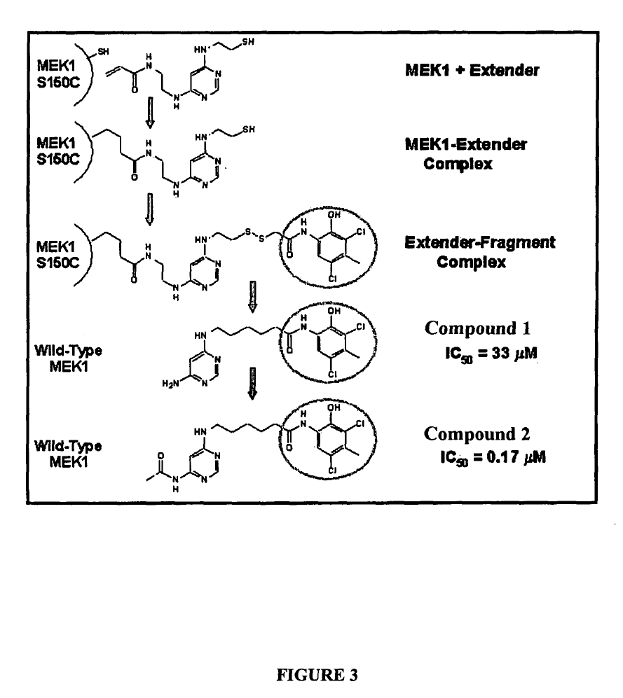

In particular, when Tethering with extenders are used, the binding determinant from the extender can be merged with the identified fragment to make a conjugate compound that is equivalent or better than a lead compound derived from traditional high-throughput screening. FIG. 3 illustrates one example of such a conjugate compound in which subsequent optimization led to a nanomolar kinase inhibitor. As shown, a cysteine mutant of MEK1 (S150C) was made that placed a thiol at the bottom of the ATP binding pocket. A pyrimidine extender that had been previously been identified as a fragment that had binding affinity for ATP binding pocket of kinases was used to form a MEK1-extender covalent complex. This complex was then used to identify a fragment that binds to the adaptive binding site that was then merged with the binding determinant from the pyrimidine extender to yield a 33 μM MEK1 inhibitor (compound 1). Acylation of the amine resulted in a 170 nM MEK1 inhibitor (compound 2). Other potent inhibitors that resulted from simple modification of compound 1 include the following compounds which inhibits MEK1 with IC₅₀'s of 80 nM, 50 nM, 30 nM, and 10 nM respectively.

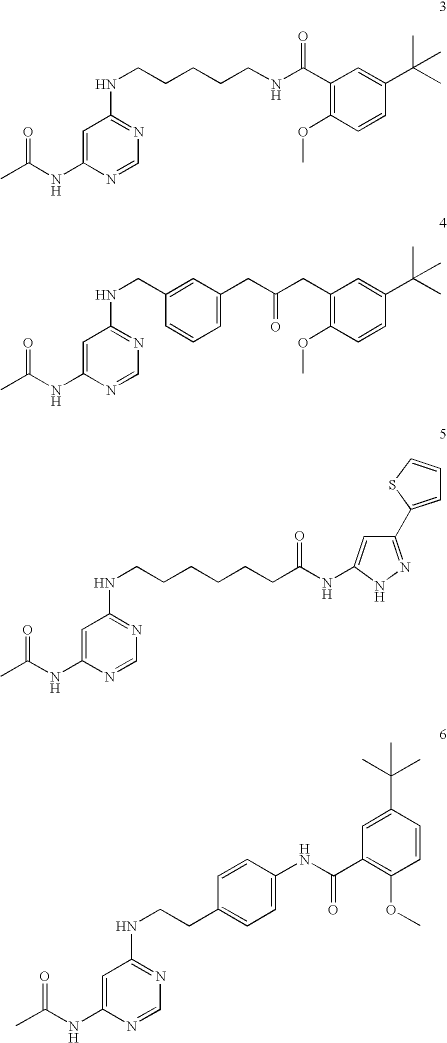

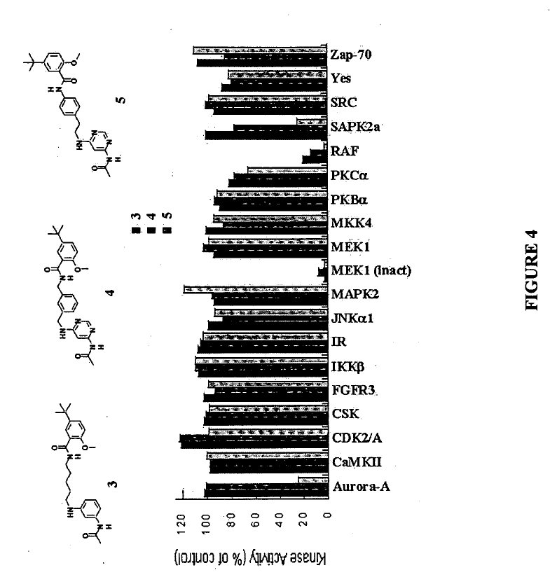

Notably, the resulting submicromolar MEK1 inhibitors all preferentially inhibit the inactive form. No inhibition of the active form of MEK1 was observed at concentrations of compounds 2-6 at 10 μM (and ATP concentrations of 50 μM). In addition, these compounds also showed remarkable specificity for compounds that have yet to be optimized. For example, neither compounds 1 nor 2 inhibit Raf kinase at concentrations that inhibit MEK1 completely. In addition, when the most potent of the above compounds (3, 4 and 6) were tested in a panel of kinases, as shown in FIG. 4, these compounds were also very specific for the inactive conformation of MEK1. Only RAF showed any significant inhibition. However, because RAF is a kinase that is immediately upstream from MEK1, inhibition of RAF may also be therapeutically desirable.

Further details of the invention are illustrated in the following non-limiting examples.

### EXAMPLE 1

Construction and Expression of EGFR1 and Lck Variants

Wild-type human Lck and wild-type human EGFR1 were cloned by RT-PCR from poly(A)+ enriched mRNA from Jurkat cells and A431 cells, respectively. Jurkat cells were grown in suspension in 30 mL of medium containing 10% fetal bovine serum (FBS), at a concentration of 8.4×107 cells/mL. Approximately 40% of the Jurkat cells were put into an Eppendorf tube and pelleted. Adherent A431 cells were grown in DMEM containing 4 mM glutamine, 1.5 g/L sodium bicarbonate, 4.5 g/L glucose, and 10% FBS. A T75 monolayer was trypsinized and resuspended in 1× phosphate buffered saline. 28 mL of A431 cells at 5.6×106 cells/mL were pelleted into a second Eppendorf tube.

The RNA was isolated from each type of cells as follows. The pelleted cells were lysed with 1 mL Tri Reagent, microfuged at 15K rpm for 10 min at 4° C., and the supernatant was transferred into a new tube. Chloroform (400 μL) was added to the tube, which was vortexed, allowed to stay at room temperature for 5 min, and then microfuged for 15 min at 4° C. The aqueous phase was transferred to a new tube, to which a half-volume of 2-propanol was added. The tube was vortexed, allowed to stay at room temperature for 5 min, and then microfuged for 10 min at 4° C. The resulting RNA pellet was resuspended in 200 μL deionized water. Poly(A)+ mRNA was purified using an Oligotex purification kit (QIAgen), and stored at −20° C.

The first strand of cDNA was obtained by reverse transcription from the poly(A)+ mRNA as follows. Oligonucleotides corresponding to SEQ ID NO:1 and SEQ ID NO:2 were used as a reverse transcriptase primer for Lck and EGFR, respectively.

| | |
|---|---|
| AGGGCCTCTCAAGGCCTCCTC | SEQ ID NO:1 |
| AGTTGGAGTCTGTAGGACTTGGC | SEQ ID NO:2 |

Reactions containing 4 μL reverse transcriptase primer, 5 μL poly(A)+ mRNA, and 13 μL deionized water were annealed by heating to 70° C. for 10 min, and then chilled on ice. Two microliters of reverse transcriptase (Powerscript) were added to a mixture of 8 μL 5× first strand buffer, 4 μL dNTPs, and 4 μL DTT (100 mM).

The reverse transcription reactions containing the first strand of the cDNA were each next used in a polymerase chain reaction. Oligonucleotides SEQ ID NO:3 and SEQ ID NO:4 were used as 5′ and 3′ PCR primers for Lck, respectively, and oligonucleotides SEQ ID NO:5 and SEQ ID NO:6 were used as 5′ and 3′ PCR primers for EGFR, respectively.

| | |
|---|---|
| CTAGGATATCCTCGAGCAAGCCGTGGTGGGAGGACGAG | SEQ ID NO:3 |
| CTAGGATATCAAGCTTTTCAGTCCTCCAGCACACTGCG | SEQ ID NO:4 |
| CAG | |
| CTAGGATATCCTCGAGCGCTCCCAACCAAGCTCTCTTG | SEQ ID NO:5 |
| AG | |
| CTAGGATATCAAGCTTTTCATTTGGAGAATTCGATGAT | SEQ ID NO:6 |
| CAACTCAC | |

One microliter of the first strand cDNA reaction was added to 1 μL of PCR primers, 1 μL 25 mM dNTPs, 5 μL 10×PFU buffer, 0.5 μL DNA polymerase (2:1 KlenTaq:Pfu-turbo (vol/vol)), and 41.4 μL deionized water. The resulting PCR reactions were heated to 94° C. for 4 min, and then cycled 35 times as follows: 94° C. for 30 sec, 57° C. for 30 sec, and 72° C. for 1 min 45 sec. Finally the reactions were allowed to remain at 72° C. for 8 min, and then held at 4° C. until the following cloning step.

The resulting duplex cDNA was cloned into an RSETB expression vector as follows. PCR products were purified on a QIAgen miniprep column, digested in a 80 μL volume with XhoI and HindIII in 1× Buffer2/BSA (New England Biolabs) for 2 hr at 37° C. Five micrograms of pRSETB were also digested by XhoI and HindIII in the same manner. The resulting digestion products were also purified on a QIAgen miniprep column, and then used in ligation reactions. All ligation reactions (Boehringer Rapid Ligation kit) contained 1 μL of purified vector, and 1-2 μL of insert, and were performed according to the manufacturer's instructions. The ligated reactions (2 μL) were transformed into Top10F′ cells, and 1/10 of the transformed cells were spread onto LB/amp plates (100 μg/mL). Resulting colonies were screened for insert by PCR, using the same reaction conditions described above, and forward and reverse primers SEQ ID NO:7 and SEQ ID NO:8, respectively; positive clones were verified by sequencing.

| | |
|---|---|
| GACCACAACGGTTTCCCTCTAG | SEQ ID NO:7 |
| GTTATTGCTCAGCGGTGGCAGC | SEQ ID NO:8 |

The resulting wild-type Lck pRSETB construct, expressing residues 231-496 of SEQ ID NO:9 was altered to express a mutant Lck construct having S323 mutated to cysteine. The mutation was designed to allow covalent attachment of a small molecule extender by introducing a cysteine residue into the target kinase in a position analogous to EGFR1 C797 of SEQ ID NO:10. The resulting EGFR1 pRSETB construct encoded residues 698-970 of SEQ ID NO:10.

| | |
|---|---|
| MGCGCSSHPE DDWMENIDVC ENCHYPIVPL DGKGTLLIRN GSEVRDPLVT | SEQ ID NO:9 |
| YEGSNPPASP LQDNLVIALH SYEPSHDGDL GFEKGEQLRI LEQSGEWWKA | |
| QSLTTGQEGF IPFNFVAKAN SLEPEPWFFK NLSRKDAERQ LLAPGNTHGS | |
| FLIRESESTA GSFSLSVRDF DQNQGEVVKH YKIRNLDNGG FYISPRITFP | |
| GLHELVREYT NASDGLCTRL SRPCQTQKPQ KPWWEDEWEV PRETLKLVER | |
| LGAGQFGEVW MGYYNGHTKV AVKSLKQGSM SPDAFLAEAN LMKQLQHQRL | |
| VRLYAVVTQE PIYIITEYME NGSLVDFLKT PSGIKLTINK LLDMAAQIAE | |
| GMAFIEERNY IHRDLRAANI LVSDTLSCKI ADFGLARLIE DNEYTAREGA | |
| KFPIKWTAPE AINYGTFTIK SDVWSFGILL TEIVTHGRIP YPGMTNPEVI | |
| QNLERGYRMV RPDNCPEELY QLMRLCWKER PEDRPTFDYL RSVLEDFFTA | |
| TEGQYQPQP | |
| MRPSGTAGAA LLALLAALCP ASRALEEKKV CQGTSNKLTQ LGTFEDHFLS | SEQ ID NO:10 |
| LQRMFNNCEV VLGNLEITYV QRNYDLSFLK TIQEVAGYVL IALNTVERIP | |
| LENLQIIRGN MYYENSYALA VLSNYDANKT GLKELPMRNL QEILHGAVRF | |
| SNNPALCNVE SIQWRDIVSS DFLSNMSMDF QNHLGSCQKC DPSCPNGSCW | |
| GAGEENCQKL TKIICAQQCS GRCRGKSPSD CCHNQCAAGC TGPRESDCLV | |
| CRKFRDEATC KDTCPPLMLY NPTTYQMDVN PEGKYSFGAT CVKKCPRNYV | |
| VTDHGSCVRA CGADSYEMEE DGVRKCKKCE GPCRKVCNGI GIGEFKDSLS | |
| INATNIKHFK NCTSISGDLH ILPVAFRGDS FTHTPPLDPQ ELDILKTVKE | |
| ITGFLLIQAW PENRTDLHAF ENLEIIRGRT KQHGQFSLAV VSLNITSLGL | |
| RSLKEISDGD VIISGNKNLC YANTINWKKL FGTSGQKTKI ISNRGENSCK | |
| ATGQVCHALC SPEGCWGPEP RDCVSCRNVS RGRECVDKCN LLEGEPREFV | |
| ENSECIQCHP ECLPQAMNIT CTGRGPDNCI QCAHYIDGPH CVKTCPAGVM | |
| GENNTLVWKY ADAGHVCHLC HPNCTYGCTG PGLEGCPTNG PKIPSIATGM | |
| VGALLLLLVV ALGIGLFMRR RHIVRKRTLR RLLQERELVE PLTPSGEAPN | |
| QALLRILKET EFKKIKVLGS GAFGTVYKGL WIPEGEKVKI PVAIKELREA | |
| TSPKANKEIL DEAYVMASVD NPHVCRLLGI CLTSTVQLIT QLMPFGCLLD | |
| YVREHKDNIG SQYLLNWCVQ IAKGMNYLED RRLVHRDLAA RNVLVKTPQH | |
| VKITDFGLAK LLGAEEKEYH AEGGKVPIKW MALESILHRI YTHQSDVWSY | |
| GVTVWELMTF GSKPYDGIPA SEISSILEKG ERLPQPPICT IDVYMIMVKC | |
| WMIDADSRPK FRELIIEFSK MARDPQRYLV IQGDERMHLP SPTDSNFYRA | |
| LMDEEDMDDV VDADEYLIPQ QGFFSSPSTS RTPLLSSLSA TSNNSTVACI | |
| DRNGLQSCPI KEDSFLQRYS SDPTGALTED SIDDTFLPVP EYINQSVPKR | |
| PAGSVQNPVY HNQPLNPAPS RDPHYQDPHS TAVGNPEYLN TVQPTCVNST | |
| FDSPAHWAQK GSHQISLDNP DYQQDFFPKE AKPNGIFKGS TAENAEYLRV | |
| APQSSEFIGA | |

Mutagenesis of the Lck pRSETB construct was performed by PCR using sense and antisense oligonucleotide of SEQ ID NO:11 and SEQ ID NO:12, respectively.

| | |
|---|---|
| CATGGAGAATGGGTGTCTAGTGGATTTTC | SEQ ID NO:11 |
| GAAAATCCACTAGACACCCATTCTCCATG | SEQ ID NO:12 |

The mutagenesis PCR reactions contained 1 μL (approximately 300 ng) of methylated pRSETB plasmid isolated from bacteria transformed by pRSETB encoding wild-type Lck (residues 231-496), 2.5 μL each of sense and antisense primers (10 μM stock concentration), 1 μL dNTPs (12.5 mM stock concentration), 5 μL Pfu-turbo buffer, 37 μL deionized water, and 1 μL Pfu-turbo polymerase. PCR reactions were heated to 95° C. for 1 min, and then cycled three times through 95° C. for 30 sec, 55° C. for 1 min, and 68° C. for 3 min. Finally the reactions were allowed to remain at 68° C. for 10 min. DpnI (1 mL) was used to digest the methylated parent plasmid, and 1 μL of the digest containing the in vitro synthesized, unmethylated, intact linear PCR product, including the introduced mutation, was transformed into Top10F′ cells, where the PCR product was ligated to produce the pRSETB plasmid encoding mutant Lck.

For expression in insect cells, the pRSETB constructs expressing wild-type EGFR1 residues 698-970 and the Lck mutant were subcloned into a pFastbacHTa vector (GIBCO-BRL), such that the resulting encoded proteins contained a (His)₆ tag on their N-termini. 5′ PCR primers SEQ ID NO:13 and SEQ ID NO:14 were used to amplify Lck and EGFR1 from the corresponding pRSETB constructs, respectively; each contain an NcoI site.

| | |
|---|---|
| CTAGGATATCCCATGGGCAAGCCGTGGTGGGAGGAC | SEQ ID NO:13 |
| GAG | |
| CTAGGATATCCCATGGCTCCCAACCAAGCTCTCTTG | SEQ ID NO:14 |
| AG | |

An EGFR1 construct having a (His)₆ tag on its C-terminus instead of its N-terminus was constructed to assist in Ni-NTA binding of the expressed protein. Furthermore, as the first EGFR1 construct (residues 698-970 of SEQ ID NO:10) was not active, a longer version was produced. Two cloning steps were performed. Firstly, the region spanning BamHI-HindIII was removed from the polylinker of the plasmid pFastBac1, and replaced with an annealed oligonucleotide duplex of SEQ ID NO:15 and SEQ ID NO:16 encoding for a (His)₆ tag. This replacement into the pFastBac1 vector obliterates the BamHI and HindIII sites, and introduces an NcoI site and a new HindIII site to produce the plasmid pFastBac1C-termHis.

| | |
|---|---|
| GATCCTCCGAAACCATGGCTCGAGGCGGCCGCAAGCT | SEQ ID NO:15 |
| TGATATCCCAACGACCGAAAACCTGTATTTTCAGGGC | |
| CATCACCATCACCATCACTAGC | |
| AGCTGCTAGTGATGGTGATGGTGATGGCCCTGAAAAT | SEQ ID NO:16 |
| ACAGGTTTTCGGTCGTTGGGATATCAAGCTTGCGGCC | |
| GCCTCGAGCCATGGTTTCGGAG | |

Secondly, a longer version of EGFR1 encoding residues 670-988 of SEQ ID NO:10 was subcloned from the EGFR1 FastBacHTa construct described above by amplification of the EGFR1 coding sequence using primers to extend the coding sequence at the N- and C-termini. The primers used for this purpose correspond to SEQ ID NO:17 and SEQ ID NO:18. The resulting DNA was inserted into the pFastBac1C-termHis plasmid so as to replace the NcoI-HindIII segment. The resulting construct encoded for residues 670-988 of SEQ ID NO:10, and contained a (His)₆ tag only on the C-terminus.

| | |
|---|---|
| GGTACCCATGGGAAGGCGCCACATCGTTCGGAAGCGC | SEQ ID NO:17 |
| ACGCTGCGGAGGCTGCTGCAGGAGAGGGAGCTTGTGG | |
| AGCCTCTTACA | |
| GGATCAAGCTTTTCAATGCATTCTTTCATCCCCCTGA | SEQ ID NO:18 |
| ATGACAAGGTAGCGCTGGGGGTCTCGGGCCATTTTGG | |
| A | |

FastBac plasmids described above encoding the kinases of interest were transformed into DH10Bac cells to construct recombinant bacmids by transposition. Specifically, approximately 500 ng pFastBac in 1 μL was added to 49 μL 1×KCM (10 mM Tris-HCl pH 7.7, 120 mM KCl, 20 mM NaCl, 0.1% Triton X-100) and 50 μL PEG/DMSO competent cells. The cells were allowed to sit for 15 min at 4° C., and then for 10 min at room temperature. SOC (900 μL) was then added to the cells, which were shaken 4 hr at 37° C. Two hundred microliters of the cell mixture was plated onto LB-agar plates containing 50 μg/mL kanamycin, 7 μg/mL gentamycin, 10 μg/mL tetracyclin, 100 μg/mL Bluo-Gal, and 40 μg/mL IPTG. Plates were grown for 2 d at 37° C., after which white colonies were picked for sequence verification.

The resulting recombinant bacmids expressing the Lck S323 mutant and EGFR1 residues 670-988 were transfected into Sf9 cells for preparation of recombinant baculovirus. Transfection of the bacmids, and harvest and storage of the recombinant baculovirus were performed according to the manufacturer's instructions (GibcoBRL).

Expression and Purification

Recombinant baculovirus was used to express the EGFR1 and mutated Lck constructs in High Five insect cells. High Five insect cells (Invitrogen) were grown to 2×106 cells/mL, and then 50 mL of High Five cells were infected with 0.5 mL virus. Standard time and temperature of induction variation was used to optimize expression conditions. Typically, the infected High Five cells were grown for 2d at 27° C.

Insect cells were pelleted and washed with 5 mL PBS; cells were then lysed with 1 mL mammalian protein extraction reagent (MPER) solution (23.5 mL MPER (Pierce), 1.5 mL 5M NaCl, 35 μL 14.3 M β-mercaptoethanol, 250 mL protease inhibitor-EDTA), rocking end over end for 20 min at 4° C. Lysate was placed in a tube and spun for 15 min at 4° C. (15K, Sorval SS-34 rotor). The aqueous layer between the pellet and the lipid layer was transferred to a fresh tube, and sufficient 50% glycerol was added to a final concentration of 10% glycerol; the solution was stored at −20° C.

N-terminally (His)₆-tagged Lck S323C mutant protein was purified from cell lysates using standard protein chromatography techniques. Specifically, the Lck S323C was purified on a 6 mL Ni-NTA-agarose column. The column was rinsed in deionized water at 2 mL/min, and then equilibrated in binding buffer (pH 8.0) containing 50 mM NaH₂PO₄, 0.5 M NaCl, and 5 mM β-mercaptoethanol. 50 mL of lysate was added to 250 mL of the binding buffer and loaded onto the column at 4 mL/min. The column was washed at 2 mL/min first with binding buffer containing no imidazole, and then with binding buffer containing 10 mM imidazole. Finally, the protein was eluted at 2 mL/min with binding buffer containing 200 mM imidazole.

C-terminally (His)₆-tagged EGFR1 (residues 670-988) was expressed and purified on Ni-NTA-agarose as described above for Lck mutant. However, for EGFR, it was necessary to follow the purification on the Ni-NTA column by purification on an S-sepharose column (ion-exchange). A 5 mL SP Sepharose FF (cation exchange) column was equilibrated with buffer containing 20 mM Tris pH 7.5, 10 mM NaCl and 5 mM β-mercaptoethanol. Three milliliters EGFR1 diluted into 87 mL of a solution of 20 mM Tris pH 7.5, 10 mM NaCl and 5 mM β-mercaptoethanol was loaded onto the column at 2 mL/min. Next, a gradient from 0-100% Buffer B (20 mM Tris pH 7.5, 1.0 M NaCl, 5 mM β-mercaptoethanol) was run in 45 min, and the eluate collected in fractions. The EGFR1 elutes at about 50% buffer B, corresponding to 0.5 M NaCl.

### EXAMPLE 2

Construction and Expression of MEK1 Variants

The amino acid sequence of MEK1 is shown here as SEQ ID NO:19.

| | |
|---|---|
| MPKKKPTPIQ LNPAPDGSAV NGTSSAETNL EALQKKLEEL ELDEQQRKRL | SEQ ID NO:19 |
| EAFLTQKQKV GELKDDDFEK ISELGAGNGG VVFKVSHKPS GLVMARKLIH | |
| LEIKPAIRNQ IIRELQVLHE CNSPYIVGFY GAFYSDGEIS ICMEHMDGGS | |
| LDQVLKKAGR IPEQILGKVS IAVIKGLTYL REKHKIMHRD VKPSNILVNS | |
| RGEIKLCDFG VSGQLIDSMA NSFVGTRSYM SPERLQGTHY SVQSDIWSMG | |
| LSLVEMAVGR YPIPPPDAKE LELMFGCQVE GDAAETPPRP RTPGRPLSSY | |
| GMDSRPPMAI FELLDYIVNE PPPKLPSGVF SLEFQDFVNK CLIKNPAERA | |
| DLKQLMVHAF IKRSDAEEVD FAGWLCSTIG LNQPSTPTHA AGV | |

The entire coding sequence of MEK1 was subcloned into the expression plasmid pGEX-6P-1 (Invitrogen) using 5′ and 3′ PCR primers (SEQ ID NO:20 and SEQ ID NO:21, respectively), along with a commercially available MEK1 cDNA (Mek1 cDNA in pUSEamp, Upstate #21-106) as a PCR template.

| | |
|---|---|
| 5′-BamHI | |
| CGCGCGGATCCATGCCCAAGAAGAAGCCGACGCCCAT | SEQ ID NO:20 |
| CCAGC | |
| 3′-Xhol | |
| CGTAGCTCGAGTCAGGTACCGGCAGCGTGGGTTGGTG | SEQ ID NO:21 |
| TGCTGGG | |

The resulting amplified MEK1 DNA was subcloned into pGEX-6P-1 at BamHI and XhoI. The resulting plasmid, pMek1-001, encodes a GST-MEK1 fusion protein in which the MEK1 portion contains a 14 amino acid insertion between residues M1 and P2 of the GenBank reported sequence, as well as three single amino acid substitutions from the GenBank reported sequence: M274L G392S, and V393T, numbering relative to SEQ ID NO:1. Following cleavage of this fusion protein with Precission protease (Amersham Biosciences), the liberated MEK1 protein contains an additional five non-native amino (GPLGS) acids at the amino terminus.

MEK1 Constructs

The surface accessibility of native cysteines was assessed by mass spectrometry, according to their reactivity with cystine in the presence of 0-16 mM P-mercaptoethanol. Of the six naturally occurring cysteines, C207, C277, and C341 were determined to be reactive cysteines and were “scrubbed”. In addition, a cysteine was introduced in a location (SISOC) analogous to that of C797 of EGFR1. All mutations have been introduced using long-range PCR with a pair of complementary oligonucleotides containing the desired mutation.

The oligos for making the constructs were:

| | |
|---|---|
| C121S-s | |
| GGTGCTGCATGAGTCCAACTCCCCGTACATAG | SEQ ID NO:22 |
| C142S-s | |
| GCGAGATCAGCATCTCCATGGAGCACATGGATG | SEQ ID NO:23 |
| S150C-s | |
| CATGGATGGTGGGTGCTTGGATCAAGTGCTG | SEQ ID NO:24 |
| C207S-s | |
| GGGAGATCAAACTCTCCGATTTTGGGGTCAG | SEQ ID NO:25 |
| C207A-s | |
| GGGAGATCAAACTCGCCGATTTTGGGGTCAG | SEQ ID NO:26 |
| S218D, S222D-s | |
| CGGGCAGCTAATTGACGACATGGCCAACGACTTCGTG | SEQ ID NO:27 |
| GGAACAAGG | |
| S218D, S222D-s | |
| CGGGCAGCTAATTGACGACATGGCCAACGACTTCGTG | SEQ ID NO:28 |
| GGAACAAGG | |
| C277S-s | |
| GAGCTGCTGTTTGGATCCCAGGTGGAAGGAG | SEQ ID NO:29 |
| C341S-s | |
| GGATTTTGTGAATAAGTCCTTAATAAAGAACCCTG | SEQ ID NO:30 |
| C341M-s | |
| GGATTTTGTGAATAAGATGTTAATAAAGAACCCTG | SEQ ID NO:31 |
| C376S-s | |
| GACTTCGCAGGCTGGCTCTCCTCCACCATTGGGCTTA | SEQ ID NO:32 |
| ACC | |

Expression of Recombinant MEK1 and Mutants

A frozen glycerol stock of E. coli (Rosetta DE3 competent cells from Novagen) containing the desired pGEX-MEK1 construct is used to inoculate 50 mL 2xYT media containing 150 μg/mL ampicillin and 30 μg/mL chloramphenicol; the resulting culture is grown overnight at 37° C. A portion of the overnight culture (10-15 mL) is then used to inoculate 1.5 L 2xYT media containing 150 μg/mL ampicillin and 30 μg/mL chloramphenicol, and the culture is grown at 37° C. until OD₆₀₀≈0.7-1.0. At this point, the cultures are chilled at 4° C. for 30-60 min; after chilling, IPTG is added to 0.2 mM, and cultures are incubated overnight at room temperature with shaking at 225 rpm (20-22° C.).

Cells are harvested by centrifugation at 5000 rpm, media is discarded, and the pellet is resuspended in 50 mL freshly made lysis buffer (1× phosphate buffered saline (PBS), 400 mM KCl, 1 M urea, 1 tablet Complete Protease Inhibitor Cocktail, 1% (v/v) aprotinin, DNase I (100 units/mL)). Cells are kept cold during the resuspension procedure, and immediately after the cells are resuspended, phenylmethyl sulfonyl fluoride (PMSF) is added to a final concentration of 2 mM. Cells are lysed by passing through a micro-fluidizer four separate times. Lysate is kept on ice, and immediately spun at 16,000 rpm at 4° C. for 30 min. While the lysate is spinning, a glutathione agarose column is equilibrated with Wash Buffer #1 (1×PBS, 400 mM KCl, 1 M urea). Supernatant is removed from the spun lysate, and immediately loaded onto the equilibrated column at 2-3 mL/min. The column is washed first with Wash Buffer #1 until the OD₂₈₀ drops to a baseline absorbance level, and then with Wash Buffer #2 (1×PBS, 400 mM KCl) for several minutes to remove the urea. The bound GST-MEK1 fusion protein is eluted with Elution Buffer (20 mM HEPES pH 8.4, 100 mM KCl, 10 mM glutathione, 1 mM DTT). The column can be regenerated by stripping with 6 M guanidine-HCl and washing with DI water after stripping. Next, GST is cleaved off the fusion protein by addition 60 μL Prescission Protease (Amersharn Biosciences); the digest reaction is transferred into 10,000-14,000 mwco dialysis tubing and dialyzed against 4 L of 20 mM HEPES pH 7.4, 150 mM NaCl, 1 mM DTT overnight at 4° C.

Subsequently the digest reaction is removed from the dialysis tubing, and spun at 16,000 rpm at 4° C. for 30 min. While the digest reaction is spinning, a glutathione agarose column is washed with Wash Buffer #3 (20 mM HEPES pH 7.4, 150 mM NaCl, 1 mM DTT). The supernatant is loaded onto the equilibrated column at 1-3 mL/min, and then the column is washed with Wash Buffer #3 until the OD₂₈₀ drops to baseline. Flow-through is collected until baseline is reached. The flowthrough is then mixed 1:1 with Dilution buffer (20 mM HEPES pH 8.4, 1 mM DTT), to make a solution that is 20 mM HEPES pH 8.0, 75 mM NaCl. A Q-Sepharose column connected in series with a prepacked 5 mL glutathione agarose column is equilibrated with Low Salt Buffer. # (20 mM HEPES pH 8.0, 75 mM NaCl, 1 mM DTT). The diluted flowthrough is loaded onto the equilibrated Q-Sepharose column at 1-3 mL/min, and the resulting flowthrough is collected. After the entire sample is loaded, the column is washed with Low Salt buffer #1 (20 mM HEPES pH 8.0, 75 mM NaCl, 1 mM DTT), and the flowthrough containing MEK1 is collected until the OD₂₈₀ reaches baseline. Bound protein (GST and impurities) is eluted by washing the column with High Salt buffer #1 (20 mM HEPES pH 8.0, 750 mM NaCl, 1 mM DTT), and collected for analysis.

The flowthrough containing MEK1 is then mixed with saturated ammonium sulfate solution (3.9 M), to a final concentration of 1.2 M ammonium sulfate. The resulting solution is then loaded at 2-3 mL/min onto an HIC phenyl-Sepharose column that has been equilibrated with High Salt Buffer #2 (20 mM HEPES pH 7.4, 1.2 M ammonium sulfate). After loading, the column is washed with High Salt Buffer #2 until the OD₂₈₀ drops to baseline. A linear gradient is run from 20 mM HEPES pH 7.4, 1.2 M ammonium sulfate to 20 mM HEPES pH 7.4 with no ammonium sulfate over 30 min, and 4 mL fractions are collected. The fractions are run on a gel to determine which fractions to pool. The pooled fractions are then dialyzed overnight at 4° C. against 4 L of 20 mM HEPES pH 7.4, 150 mM NaCl, in the absence of DTT. Finally, the pooled fractions are dialyzed again against 2 L of 20 mM HEPES pH 7.4, 150 mM NaCl for 2-4 hr. The dialyzed protein is quantitated, divided into aliquots and stored frozen at −80° C. One absorbance unit at 280 nm is equivalent to a concentration of 1.86 mg/mL, and 1 μg of MEK1 is equivalent to 22.8 pmol, as MEK1 has a MW of 43,832.

### EXAMPLE 3

Activity Assays

MEK1 ELISA Assay

Phosphorylation of ERK2 by MEK1 is measured for two reaction formats. The first reaction format is a Raf→MEK1→ERK2 cascade where constitutively active truncated Raf1, inactive MEK1, inactive biotinylated ERK2, and dephosphorylated MBP (Myelin Basic Protein) are present. The second reaction format uses activated MEK1, biotinylated ERK2, and dephosphorylated MBP in the absence of Raf Results can be compared to determine whether a compound preferentially inhibits the inactive conformation of MEK1 over the active conformation of MEK1.

Both reaction formats are run in the presence and absence of compounds, and use ELISA as a readout of the extent of phosphorylation of the biotinylated ERK2. For either format, where the activity of a potential inhibitor is unknown, generally two sets of experiments are run. In the first set, three final concentrations of compound are used, e.g., 50 μM, 10 μM and 2 μM. In the second set, nine concentrations of the compound with 2 fold dilutions are used to determine the IC₅₀ for the compound; the concentrations of the compound used depend on the activity observed in the three-point experiment. Typical stock concentrations of a moderately active compound in a 9-point experiment are 1 mM, 0.5 mM, 0.25 mM, 0.125 mM, 62.5 μM, 31.2 μM, 15.6 μM, 7.8 μM and 3.9 μM. The corresponding final concentrations of compound in the phosphorylation reaction are 20 μM, 10 μM, 5 μM, 2.5 μM, 1.25 μM, 0.625 μM, 0.312 μM, 0.156 μM, and 0.078 μM. For less active compounds, the most concentrated final concentration of compound would be 200 μM, and for more active compounds, the most concentrated final concentration of compound would be 2 μM. Biotinylation of ERK and preparation of ELISA capture plates are described below, followed by conditions for the two reaction formats, and details on post-reaction processing.

Inactive ERK2 (Cell Signaling #6082) is biotinylated as follows. Twenty-five microliters of 10×PBS and 200 μL of 50 mM carbonate buffer pH 9.0 are added to 250 μL of ERK2 at 2 mg/mL; the resulting solution is kept on ice for 10 min. Next, sulfo-NHS-LC-LC-biotin (Pierce) is freshly dissolved in solution to a final concentration of 2 mg/mL, and 10 μL of the biotin solution is added immediately to the ERK2 solution. The resulting reaction is incubated at room temperature for 1 hr, after which 100 μL of 3 M ethanolamine is added to quench the reaction. Five hundred microliters of the quenched reaction are loaded onto a Nap5 column, discarding the flowthrough. The remaining 85 μL of the quenched reaction are then loaded onto the same column, while collecting the flowthrough, followed by 715 μL Tris-buffered saline (1×TBS: 10 mM Tris pH 7.5, 150 mM NaCl), while continuing to collect the flowthrough. Recovery of biotinylated, inactive ERK2 from the Nap5 column can be monitored by Bradford assay (Bio-Rad Protein Assay Dye Reagent #500-0006) according to manufacturer's instructions. Biotin-ERK2 is stored at −20° C. in 1×TBS containing 10% glycerol.

Avidin-coated capture plates are prepared by adding 100 μL of NeutrAvidin (Pierce #31000) in PBS at 0.040 mg/mL to each well of 96-well polystyrene plates (NUNC brand maxisorp, VWR #442404). After addition of the NeutrAvidin, the plates are covered and allowed to sit at room temperature for 2-4 hr, or overnight at 4° C. NeutrAvidin is then aspirated, and 150 μL of BLOCK solution (0.05 g/mL BSA, 1×TBS, 0.1% Tween-20) is added to each well. The plates are allowed to sit at room temperature for 0.5-2 hr, until the phosphorylation reactions are ready to be transferred to the capture plate.

Phosphorylation Cascade Reactions Using Inactive MEK1

Typical phosphorylation reactions are performed in either eppendorf tubes or 96-well plates with conical bottoms (Costar #3363). The phosphorylation reactions in this reaction format contain in a 50 μL total volume the following components: μg/mL MBP (Upstate #13-110), 150 nM biotin-ERK2, 0.7 nM Raf1 (residues 306-648, N-terminally GST-tagged, Upstate # 14-352), 10 nM MEK1, 4.5 mM MgCl₂, 100 μM NaOVO₃, 30 mM Tris HCl (pH 7.5), 120 mM NaCl, 6 mM DTT, 0.0067% Triton X-100 (vol/vol) and 50 μM ATP; all concentrations are final. Forty-five microliters of all reagents except the ATP, MgCl₂ and NaOVO₃ are added to 1 μL of stock concentrations of compound in DMSO; thus the phosphorylation reactions using inactive MEK1 contain a final amount of DMSO that is 2% by volume. Addition of 5 μL of a solution of ATP, MgCl₂, and NaOVO₃, each 10 fold higher in concentration than their respective final concentrations, starts the phosphorylation reaction. Reactions are allowed to proceed 30 min at room temperature with gentle shaking.

Phosphorylation Reactions Using Active MEK1

Typical phosphorylation reactions are performed in either eppendorf tubes or 96-well plates with conical bottoms (Costar #3363). The phosphorylation reactions in this format contain in a 50 μL total volume the following components: 20 μg/mL MBP (Upstate #13-110), 150 nM biotin-ERK2, 1 nM active MEK1 (Upstate #14-429), 4.5 mM MgCl₂, 100 μM NaOVO₃, 30 mM Tris HCl (pH 7.5), 120 mM NaCl, 6 mM DTT, 0.0067% Triton X-100 (vol/vol) and 50 μM ATP; all concentrations are final. The concentration of active MEK1 used is lower than the concentration of inactive MEK1 in the format above, in order to keep readout in the linear range. Forty-five microliters of all reagents except the ATP, MgCl₂ and NaOVO₃ are added to 1 μL of stock concentrations of compound in DMSO; thus the phosphorylation reactions using active MEK1 also contain a final amount of DMSO that is 2% by volume. Addition of 5 μL of a solution of ATP, MgCl₂, and NaOVO₃, each 10 fold higher in concentration than their respective final concentrations, starts the phosphorylation reaction. Reactions are allowed to proceed 30 min at room temperature with gentle shaking.

Post-Reaction Treatment

Post-reaction treatment is the same for both reaction formats. After reaction, the solution phase phosphate-transfer reactions are stopped by addition of 75 μL stop buffer containing 0.4 M EDTA pH 7.5, 1% BSA, 1×TBS and 0.1% Tween-20. At this point, the BLOCK is removed from the prepared avidin-coated capture plates, and a 100 μL portion of each stopped reaction is transferred to a well of the plate. Biotinylated ERK2 is captured on the surface of the avidin-coated polystyrene plate by incubation of the plate at room temperature with gentle shaking for 1-2 hr. Subsequently, the reaction mixture is aspirated and the plate is incubated with a primary polyclonal antibody (Cell Signaling #9101) that recognizes the activation loop of ERK2 phosphorylated on T202 and Y204, the antibody diluted 1000 fold in a solution containing final concentrations of 1% BSA, 1×TBS and 0.1% Tween-20 by volume. The capture plate is incubated with the primary antibody solution at room temperature with gentle shaking for 2-3 hr prior to aspiration and addition of 100 μL of the secondary antibody, which is horseradish peroxidase (HRP)-conjugated Goat anti Rabbit IgG, (Zymed #62-6120) that has been diluted 1000 fold in 1% BSA, 1×TBS, 0.1% Tween 20. The secondary antibody is incubated with the plate at room temperature for 1-2 hr with gentle shaking, the solution is aspirated, and the wells are then washed gently 3 times with 1×PBS with 0.05% Tween-20. The amount of phosphorylated ERK2 present on the capture plate is quantitated using the ImmunoPure TMB substrate kit (Pierce #34021). After the PBS is aspirated, 100 μL of a freshly made TMB/H₂O₂ solution at room temperature containing equal volumes of peroxidase substrate solution (TMB, #1854050) and H₂O₂ solution (#1854060) is added to the wells, and the plate is incubated at room temperature with gentle shaking for 5-20 min. Color development is stopped by adding 100 μL of 2.5 M H₂SO₄ to each well of the capture plate and shaking gently for 1-2 min. Absorbance of the substrate is measured at 450 nm.

EGFR1 and Lck ELISA Assay

The ELISA assay for EGFR1 and Lck are generally similar to that described above for MEK1 except that biotinylated E4Y substrate is used instead of ERK2. Typical EGFR1 or Lck kinase assays contain 0.75% BSA, 30 mM Tris pH 7.5, 30 mM MgCl₂, 18 mM MnCl₂, 45 μM Na₂VO₃, 0.5 mM DTT, 100 μM EGFR or Lck kinase, 30 μg/ml biotinylated E₄Y, and 60 μM ATP. Bound substrate/reaction product was reacted with HRP-conjugated anti-phospho-tyrosine antibody instead of sequentially with anti-phospho-p44/42 ERK1/2 antibody and HRP-conjugated anti-rabbit antibody for the MEK1 assays.

MEK1 Western Assay

The MEK1 ELISA does not distinguish between Raf inhibition and MEK1 inhibition. Therefore, a Western assay was established for independently monitoring Raf activity. This assay has a ten fold lower throughput (8-10 compounds per week) than the ELISA, but it allows for independent analyses of both MEK1 and Raf inhibition in the same assay. Briefly, assays are carried out as described for the ELISA format with the exception that ERK2 is used in place of biotinylated ERK2 and reactions are terminated with the addition of SDS-PAGE gel loading buffer. Following SDS-PAGE electrophoresis and transfer to PVDF, transfer membranes are incubated overnight with primary antibody in either TBST with 5% BSA and anti MEK1, anti phospho MEK1, or anti ERK (Cell Signaling #9122, #9121, and #9102 respectively) or TBST with 5% nonfat dry milk and anti-phospho ERK (Cell Signaling #9101). All transfer membranes are then incubated for two hours in TBST with 5% nonfat dry milk and HRP-conjugated anti-Rabbit antibody (ZyMed #62-6120) and HRP activity quantified using ECL plus (Amersham #RPN2132).

### EXAMPLE 4

Tethering

EGFR1

Tethering was performed on the inactive conformation of EGFR1 (not phosphorylated on Y745) using Cys797 as the reactive thiol. The disulfide containing monophore library was screened in pools of 10. Using 2 μM EGFR1, 500 μM library pool, and 600 μM BME, 252 compounds gave >50% conjugation to C97. These 252 compounds were re-tested as isolated compounds using 2 μM EGFR1, 50 μM discrete compound, and 600 μM BME. In this manner, 214 (85%) screening hits were confirmed. The identified ligands showed clear preference for some chemical classes (aromatic 5 and 6 carbon ring systems and aromatic 5,6 carbon heterocycles, separated from the thiol by a single methylene linker) while other chemical classes were not selected (aliphatic chains, aliphatic 5 carbon rings, and aliphatic 6 carbon rings, separated from the thiol by a 2 or 3 methylene linker). Not surprisingly, these ligands showed clear enrichment for a number of purine-like compounds, including pyrazines, pyridines, quinolines, quinoxalines, pyrazoles, thiazoles, and other substituted benzenes.

MEK1

Tethering was performed on the inactive conformation (not phosphorylated on either S223 or S227) of a MEK1 a mutant containing a cysteine at S150 (an amino acid corresponding to C797 of EGFR1). A partial library screen showed not only a very similar hit rate to that seen with the EGFR (1.4% with greater than 50% conjugation), but also a strong structural similarity to the EGFR purine pocket screening hits. Consequently, instead of repeating the entire library screen, the hits from EGFR1 were tested individually against MEK1.

### EXAMPLE 5

This example describes the synthesis of 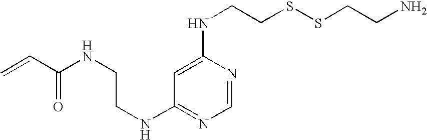
which as prepared according to Scheme 1 and the procedure below.

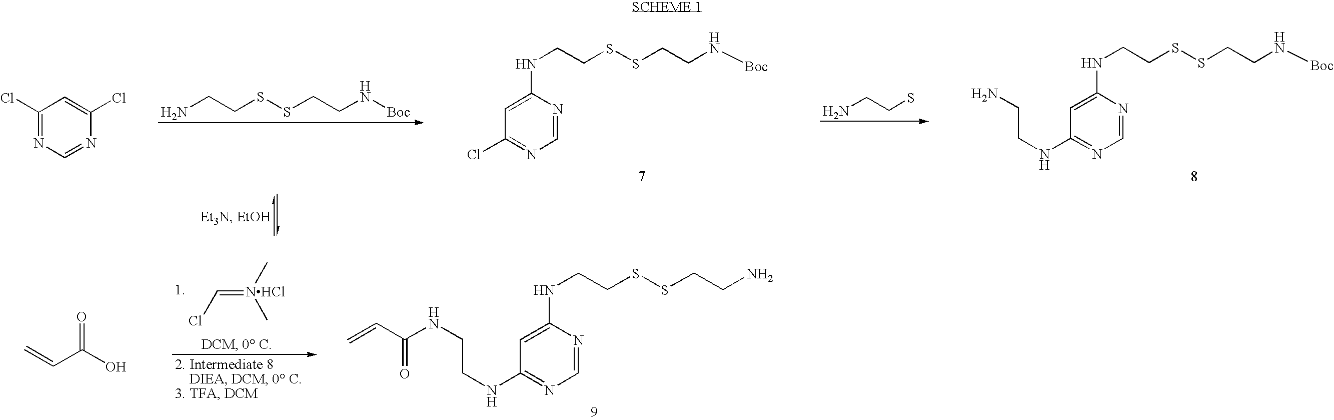

To a solution of 4,6-dichloropyrimidine (0.500 g, 3.358 mmol) in ethanol (8.3 mL) was added[2-(2-Amino-ethyldisulfanyl)-ethyl]-carbamic acid tert-butyl ester (0.706 g, 2.798 mmol) and triethylamine (0.417 mL, 2.994 mmol). The reaction mixture was refluxed overnight under N₂ (12 h). The solvent was evaporated and the crude reaction mixture was purified by silica gel chromatography (50% ethyl acetate (“EtOAc”) in hexanes) to provide intermediate 7 (0.662 g) as a white solid in 54% yield. ¹H NMR (400 MHz, CHLOROFORM-D) ppm 1.47 (s, 9H) 2.78 (d, J=13.99 Hz, 2H) 2.94 (d, J=10.94 Hz, 2H) 3.48 (m, 2H) 3.76 (s, 2H) 5.07 (s, 1H) 6.56 (s, 1H) 8.37 (s, 1H). LCMS M+1=365.

Intermediate 7 (0.200 g, 0.548 mmol) in neat ethylene diamine (5 mL) was refluxed under N₂ overnight. The reaction mixture was diluted with EtOAc and partitioned with saturated NaHCO₃. The aqueous layer was extracted with EtOAc (3×). The combined organic layers were rinsed with saturated NaCl, dried over Na₂SO₄, filtered and the solvent was evaporated, to provide intermediate 8 (0.145 g) as a light yellow solid in 68% yield. Intermediate 8 was used without purification in the next step. ¹H NMR (400 MHz, MeOD) ppm 1.42 (s, 9H) 2.79 (m, 3H) 2.87 (t, J=6.61 Hz, 3H) 3.31 (d, J=8.90 Hz, 3H) 3.54 (t, J=6.36 Hz, 3H) 5.49 (s, 1H) 7.92 (s, 1H). LCMS M+1=389.

To a solution of acrylic acid (0.012 mL, 0.172 mmol) in 1 mL dichloromethane (“DCM”) at 0° C. was added (chloromethylene) dimethyl ammonium chloride. The reaction mixture was stirred for 1 hr at 0° C. under N₂. This solution was then added dropwise to a stirred solution of intermediate 8 (0.067 g, 0.172 mmol) and N, N-diisopropylethylamine (0.061 mL, 0.343 mmol) in 1 mL DCM at 0° C. After stirring for 1 hr under N₂, the reaction mixture was diluted with DCM, rinsed with 1 M Na₂CO₃, dried over Na₂SO₄, filtered and concentrated down to a yellow residue. The crude product was deprotected with 1:1 TFA/DCM (2 mL) where TFA is trifluoroacetic acid. The mixture was stirred for 30 min and the solvent evaporated. The residue was purified using reverse phase prep. HPLC to provide the titled compound 9. ¹H NMR (400 MHz, MeOD) ppm 2.78 (m, 3H) 3.11 (m, 3H) 3.27 (s, 3H) 3.50 (m, 3H) 5.48 (m, 1H) 5.58 (s, 1H) 6.03 (d, J=5.60 Hz, 2H) 7.94 (s, 1H). LCMS M+1=343.

### EXAMPLE 6

This example describes the synthesis of 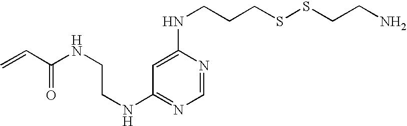
which as prepared according to Scheme 2 and the procedure below.

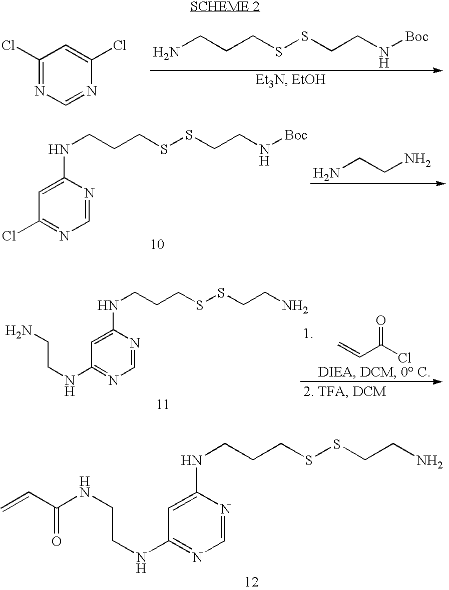

To a solution of 4,6-dichloropyrimidine (1.200 g, 8.059 mmol) in ethanol (20 mL) was added[2-(3-Amino-propyldisulfanyl)-ethyl]-carbamic acid tert-butyl ester (2.147 g, 8.059 mmol) and triethylamine (1.211 mL, 8.623 mmol). The reaction mixture was refluxed overnight under N₂ (12 hr). The solvent was concentrated under reduced pressure and the crude reaction mixture was purified by silica gel chromatography (50% EtOAc in hexanes) to provide intermediate 10 (1.978 g) as a clear oil in 78% yield. ¹H NMR (400 MHz, MeOD) ppm 1.33 (s, 9H) 1.89 (m, 1H) 2.67 (t, J=6.87 Hz, 4H) 3.23 (m, 3H) 3.40 (s, 2H) 6.40 (s, 1H) 8.12 (s, 1H). LCMS M+1=379.

Intermediate 10 (1.000 g, 2.639 mmol) in neat ethylene diamine (10 mL) was refluxed under N₂ overnight. The reaction mixture was diluted with EtOAc and partitioned with saturated NaHCO₃. The aqueous layer was extracted with EtOAc (3×). The combined organic layers were rinsed with saturated NaCl, dried over Na₂SO₄, filtered and concentrated under reduced pressure to provide intermediate 11 (0.605 g) as a white solid in 57% yield. Intermediate 11 was used without purification in the next step. ¹H NMR (400 MHz, MeOD) ppm 1.32 (s, 9H) 1.87 (m, 2H) 2.69 (m, 4H) 3.20 (m, 8H) 5.35 (s, 1H) 7.79 (s, 1H). LCMS M+1=403.

To a solution of intermediate 11 (0.100 g, 0.248 mmol) in 1 mL DCM at 0° C. was added acryloyl chloride (20.2 μL, 0.248 mmol) and N,N-diisopropylethylamine (86.5 μL, 0.497 mmol). The resulting dark brown solution was stirred at 0° C. for 30 min. The reaction was diluted with DCM, rinsed with 1 M Na₂CO₃, dried over Na₂SO₄, filtered and concentrated to a yellow solid. The crude product was deprotected with 1:1 TFA/DCM (2 mL). The mixture was stirred for 30 min and the solvent evaporated. The residue was purified using reverse phase prep HPLC to provide the titled compound 12. ¹H NMR (400 MHz, MeOD) ppm 1.94 (m, 2H) 2.72 (t, J=7.12 Hz, 2H) 2.83 (t, J=6.61 Hz, 2H) 3.18 (m, 4H) 3.35 (s, 4H) 5.57 (t, J=5.60 Hz, 1H) 5.62 (s, 1H) 6.11 (d, J=6.10 Hz, 2H) 8.01 (s, 1H). LCMS M+1=357.

### EXAMPLE 7

This example describes the synthesis of 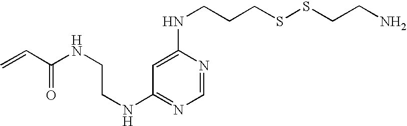
which as prepared according to Scheme 3 and the procedure below.

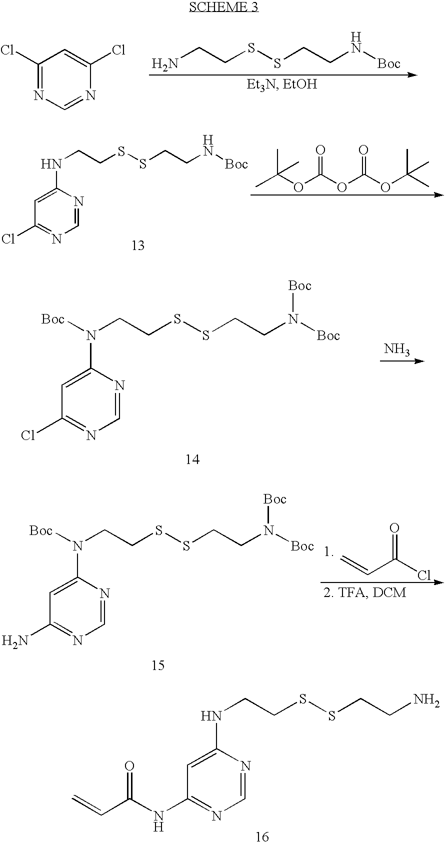

4,6-Dichloropyrimidine (2.0 g, 13.42 mmol)[2-(2-Amino-ethyldisulfanyl)-ethyl]-carbamic acid tert-butyl ester (14.77 mL, 1.0 M in DCM, 14.77 mmol), and triethylamine (9.35 mL, 67.10 mmol), were dissolved in 70 mL ethanol and heated to 85° C. for 16 h. The reaction was cooled to ambient temperature, the solvent evaporated, and the residue slurried in ethyl ether. The mixture was filtered, the filtrate concentrated and purified by flash chromatography (20% EtOAc in hexanes) yielding compound 13 (2.86 g, 7.8 mmol, 58% yield). ¹H NMR (400 MHz, CDCl₃): δ 1.47 (s, 9H), 2.78 (m, 2H), 2.95 (m, 2H), 3.48 (m, 2H), 3.75 (m, 2H), 5.14 (m, 1H), 6.56 (s, 1H), 8.37 (s, 1H). ESI-MS m/z: 365 (M+H)⁺.

Compound 13 (1.40 g, 3.84 mmol), N,N-dimethylaminopyridine (“DMAP”) (0.047 g, 0.384 mmol), and di-tert-butyl-dicarbonate (5.02 g, 23.02 mmol) were dissolved in 85 mL dry tetrahydrofuran (“THF”). The solution was refluxed for 6 h, the solvent evaporated, and the residue purified by flash chromatography (20% EtOAc in hexanes) yielding compound 14 (1.74 g, 3.74 mmol, 97% yield). ¹H NMR MHz, CDCl₃): δ 1.52 (s, 18H), 1.60 (s, 9H), 2.93 (m, 4H), 3.92 (m, 2H), 4.36 (m, 2H), 8.14 (s, 1H), 8.67 (s, 1H). ESI-MS m/z: 566 (M+H)⁺.

Compound 14 (1.74 g, 3.08 mmol) was dissolved in 100 mL 7 N NH₃ in methanol and the solution stirred in a sealed glass bomb at 90° C. for 6 days. The reaction was cooled to ambient temperature, the solvent evaporated, and the residue slurried in ethyl ether. The mixture was filtered, the filtrate concentrated and purified by flash chromatography (50% EtOAc in hexanes) yielding compound 15 (0.626 g, 1.15 mmol, 37% yield). ¹H NMR (400 MHz, CDCl₃): δ 1.51 (s, 18H), 1.56 (s, 9H), 2.91 (m, 4H), 3.91 (m, 2H), 4.28 (m, 2H), 4.87 (m, 2H), 7.15 (s, 1H), 8.34 (s, 1H). ESI-MS m/z: 546 (M+H)⁺.

Compound 15 (0.626 g, 1.15 mmol) was dissolved in 12 mL dry DCM under N₂, N,N-diisopropylethylamine (“DIEA”) (0.401 mL, 2.3 mmol) was added and the solution chilled to 0° C. on an ice bath. After stirring on ice for 20 min, acryloyl chloride (0.104 mL, 1.15 mmol) was added and the reaction was allowed to stir for an additional 30 min. The volatiles were evaporated and the residue slurried in ethyl ether. The mixture was filtered, the filtrate concentrated and redissolved in 10 mL dry DCM. TFA (10 mL) was added and the solution stirred at ambient temp for 30 min. The solvent was removed under reduced pressure and the crude residue purified by reverse-phase preparatory HPLC to afford compound 16 (0.076 g, 0.254 mmol, 22%). ¹H NMR (400 MHz, CD₃OD): δ 2.85 (m, 4H), 3.18 (m, 2H), 3.69 (m, 2H), 5.82 (m, 1H), 6.35 (m, 2H), 6.65 (s, 1H), 8.27 (s, 1H). ESI-MS m/z: 300 (M+H)⁺.

### EXAMPLE 8

This example describes the synthesis of 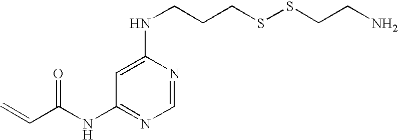
which as prepared according to the procedure of Example 7 except substituting[2-(3-Amino-propyldisulfanyl)-ethyl]-carbamic acid tert-butyl ester for [2-(2-Amino-ethyldisulfanyl)-ethyl]-carbamic acid tert-butyl ester. ¹H NMR (400 MHz, CD₃OD): δ 1.91 (m, 2H), 2.67 (m, 2H), 2.79 (m, 2H), 3.14 (m, 2H), 3.48 (m, 2H), 5.81 (m, 1H), 6.31 (m, 2H), 6.63 (s, 1H), 8.24 (s, 1H). ESI-MS m/z: 314 (M+H)⁺.

### EXAMPLE 9

This example describes Tethering with extenders on the inactive conformation of MEK1. A cysteine mutant of MEK1 S150C that also included the following mutations C207A, C277S, C376S was used for the following labelling procedure. A frozen aliquot of MEK1 (20 mM HEPES pH 7.4 150 mM NaCl) was thawed, and DTT was added to a final concentration of 2 mM. An extender, stored at a concentration of 100 mM in DMSO, was added to the protein so that the final concentration of extender was 1 mM. Subsequently, protein, reductant, and extender were incubated at 4° C. overnight, such that greater than 80% of protein was labelled with extender, as detected by mass spectrometry. The samples were injected onto an HP 1100 HPLC and chromatographed on a Protein MicroTrap (Micrhom Bioresources, Inc. # 004/25109/03) attached to a hybrid quadrupole-TOF QSTAR Pulsar i mass spectrometer (PE Sciex Instruments). The QSTAR was outfitted with a MicrolonSpray ESI source, and was operated in the positive ion mode, scanning the range of 800-1400 m/z.

After labelling, the protein-extender covalent complex was dialyzed against 7 L dialysis buffer (20 mM HEPES pH 7.4, 150 mM NaCl) overnight at 4° C. to remove unreacted extender and reductant. After checking for protein labelling again by QSTAR, the protein-extender conjugate was split into 1.1 mL working aliquots at 2 μM, frozen on dry ice/ethanol, and stored at −80° C. Depending upon the reactivity of the protein cysteine(s) and extender being used, different reaction conditions, e.g., type of reductant, concentration of reductant, reaction time, etc., can be used.

A working aliquot of the MEK1-extender covalent complex was thawed and placed on ice. A library of compounds to be screened was distributed across the wells of a 96-well plate, with each well containing a pool of 10 disulfide-containing compounds. The compounds were pooled so that each compound in the pool has a unique molecular weight, thus enabling deconvolution of the various protein-extender-compound conjugates by mass spectrometry. The library pools, stored at stock concentrations of 12.5 μM/pool in DMSO at 4° C., were thawed at room temperature for at least 30 min prior to screening. To an assay plate the following reagents were added in order: 0.86 μL of each library pool, 1 μL of 13.5 mM β-mercaptoethanol, and 25 μL of protein-extender conjugate. Thus the final screening conditions were 400 μM library pools, 500 μM β-mercaptoethanol, and 2 μM protein-extender covalent complex. The reactions were incubated at room temperature on a shaker for 1-2 hr. After reaction, samples were run on a QSTAR mass spectrometer as described above for the labelling step, in order to determine which of the library compounds reacted with the protein-extender covalent complex.

### EXAMPLE 10

This example describes the synthesis of the following compound 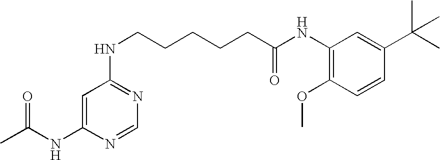
which was prepared according to Scheme 4 and the procedure below.

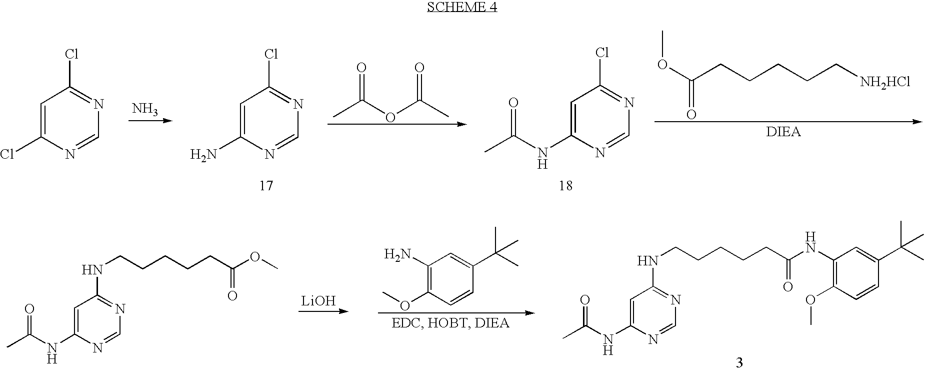

a) 4,6-Dichloropyrimidine (20.85 g, 139 mmol) and 7 N Ammonia in methanol (200 mL) were heated to 85 C in a sealed glass bomb for 16 h. The reaction was cooled to ambient temperature, the solvent evaporated, and the residue recrystalized from water yielding compound 17 (12.07 g, 93.17 mmol, 67% yield). ¹H NMR (400 MHz, DMSO-D6): δ 6.43 (s, 1H), 7.22 (s, 2H), 8.18 (s, 1H). ESI-MS m/z: 130 (M+H)⁺.

b) Compound 17 (3.30 g, 25.47 mmol) was mixed with acetic anhydride (50 mL) and refluxed for 5 h, the solvent evaporated, and the residue co-evaporated with toluene twice, yielding compound 18 (4.31 g, 25.06 mmol, 99% yield). ¹H NMR (400 MHz, DMSO-D6): δ 2.12 (s, 3H), 8.06 (s, 1H), 8.71 (s, 1H), 11.21 (s, 1H). ESI-MS m/z: 172 (M+H)⁺.

c) Compound 18 (3.03 g, 17.66 mmol), methyl 6-aminohexanoate hydrochloride (4.50 g, 24.22 mmol), and DIEA (30.76 mL, 177 mmol) were combined with n-butanol (88 mL) and refluxed on a heating mantle for 3 h. The reaction was cooled to ambient temperature, the solvent evaporated, and the residue purified by flash chromatography (80% EtOAc in hexanes) yielding compound 19 (2.63 g, 9.38 mmol, 53% yield). ¹H NMR (400 MHz, DMSO-D6): δ 1.28 (m, 2H), 1.49 (m, 4H), 2.04 (s, 3H), 2.27 (m, 2H), 3.21 (s, 1H), 3.36 (s, 1H), 3.56 (s, 3H), 7.13 (s, 1H), 7.36 (s, 1H), 8.12 (s, 1H), 10.24 (s, 1H). ESI-MS m/z: 281 (M+H)⁺.

d) Compound 19 (1.84 g, 6.57 mmol) was suspended in p-dioxane (16 mL). Lithium hydroxide (0.157g, 6.57 mmol) in water (16 mL) was added and the reaction stirred at ambient temperature for 16 h. 1N HCL (6.57 mL) was added and the solution stirred for 1 h at which point the solvent was evaporated to yield crude free acid which was taken on without further purification (2.03 g, 6.57 mmol, 99% yield). ESI-MS m/z: 267 (M+H)⁺. This free acid (0.216 g, 0.507 mmol) was mixed with 1-ethyl-(3-dimethylaminopropyl)carbodiimide hydrochloride (EDC, 0.107 g, 0.558 mmol), hydroxybenzotriazole hydrate (HOBT, 0.085 g, 0.558 mmol), and 5-tert-butyl-o-aniside (0.100 g, 0.558 mmol). DMF (3 mL) was added, followed by DIEA (0.486 mL, 2.79 mmol) and the reaction stirred at ambient temperature for 16 h. The reaction was diluted with acetonitrile (3 mL) and the crude mixture purified by reverse-phase preparatory HPLC to afford compound 3 (0.082 g, 0.151 mmol, 27%). ¹H NMR (400 MHz, CD₃OD): δ 1.07 (s, 9H), 1.27 (m, 2H), 1.51 (m, 4H), 1.99 (s, 3H), 2.24 (m, 2H), 3.10 (m, 2H), 3.63 (s, 3H), 6.40 (s, 1H), 6.69 (m, 1H), 6.91 (m, 1H), 7.79 (s, 1H), 8.12 (s, 1H) ESI-MS m/z: 456 (M+H)⁺.

### EXAMPLE 11

This example describes the synthesis of the following compound 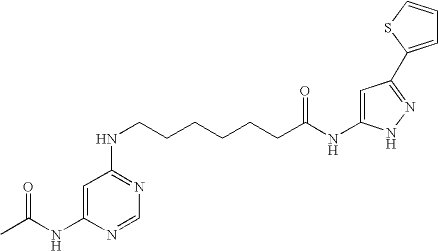
which was prepared according to the procedure below.

Boc-7-aminoheptanoic acid (4.0 g, 16.31 mmol) was dissolved in benzene (60 mL) and methanol (20 mL) was added. (Trimethylsilyl)diazomethane (2.0 M in hexanes) (16.31 mL, 32.61 mmol) was added and the solution stirred at ambient temperature for 30 min at which point the solvent was removed. The crude residue was then dissolved in 4.0 M HCL in dioxane (42 mL) and stirred for 2 h at ambient temperature at which point the solvent was removed, yielding 7-amino-heptanoic acid methyl ester (3.04 g, 15.53 mmol, 95%). ESI-MS m/z: 196 (M+H)⁺.

The titled compound was prepared according to Example 10c-d except starting with 7-amino-heptanoic acid methyl ester instead of methyl 6-aminohexanoate hydrochloride. ¹H NMR (400 MHz, CD₃OD): δ 1.32 (m, 4H), 1.57 (m, 4H), 2.04 (s, 3H), 2.91 (m, 2H), 3.15 (m, 3H), 6.43 (s, 1H), 6.91 (m, 1H), 7.23 (m, 2H), 8.19 (s, 1H). ESI-MS m/z: 428 (M+H)⁺.

### EXAMPLE 12

This example describes the synthesis of 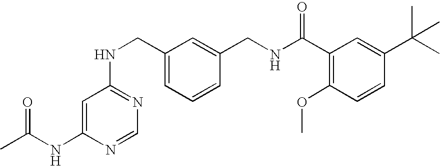
which was prepared according to Scheme 5 and the protocol below.

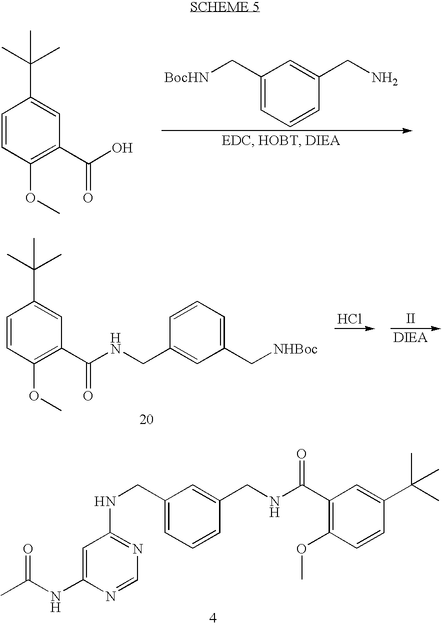

5-Tert-butyl-2-methoxybenzoic acid (0.161 g, 0.773 mmol) was mixed with EDC (0.153 g, 0.797 mmol), HOBT (0.106 g, 0.785 mmol), dissolved in 5 ml dry DMF, and 1-(N-Boc-aminomethyl)-3-(aminomethyl)benzene (0.222 g, 0.939 mmol) was added, along with DIEA (0.4 ml, 2.3 mmol). The reaction was allowed to stir at ambient temperature for 22 hours, at which point it was flooded with 50 ml EtOAc, rinsed with 2×25 ml 1 M sodium hydrogen sulfate, 2×25 ml saturated sodium bicarbonate, 25 ml brine, dried over sodium sulfate, and evaporated to dryness to yield product 20 which was used without further purification. ESI-MS m/z: 449 (M+Na)⁺.

Compound 20 (0.086 g, 0.202 mmol) was dissolved in 4 M HCl in dioxane (5 ml) and allowed to stir for 30 minutes. The solvent was removed under reduced pressure and then evaporated twice from DCM. Compound 18 (0.035 mg, 0.203 mmol) was then added, along with DIEA (0.12 ml, 0.689 mmol) and 2 ml n-butanol. The reaction was then heated to 100 C for 22 hours, at which point the reaction was flooded with EtOAc (40 ml), rinsed with 3×20 ml 1 M sodium hydrogen sulfate, 20 ml brine, dried over sodium sulfate, and evaporated to dryness. The residue was then purified by reverse-phase preparatory HPLC to afford compound 4 (0.010 g, 0.017 mmol, 9%). ¹H NMR (400 MHz, CD₃OD): δ ppm 1.31 (m, 9H) 2.18 (m, 3H) 3.92 (m, 3H) 4.60 (m, 2H) 4.71 (m, 2H) 6.48 (m, 1H) 7.08 (m, 1H) 7.24 (m, 1H) 7.34 (m, 3H) 7.55 (m, 1H) 7.97 (m, 1H) 8.35 (m, 1H). ESI-MS m/z: 462 (M+H)⁺.

### EXAMPLE 13

This example describes the synthesis of 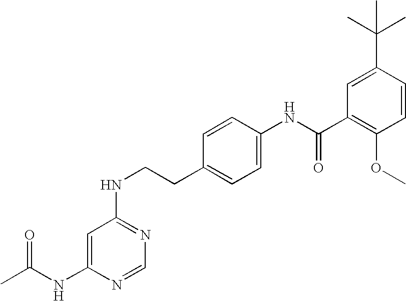
which was made according to the protocol below.

Compound 6 was prepared following the procedure of Example 12, but starting with[2-(4-amino-phenyl)-ethyl]-carbamic acid tert-butyl ester instead of 1-(N-Boc-aminomethyl)-3-(aminomethyl)benzene. The final product 6 was purified first by reverse-phase preparatory HPLC and then by silica gel chromatography, eluting first with 50:50 DCM:EtOAc, then 25:75 DCM:EtOAc, and finally eluting with pure EtOAc. ¹H NMR (400 MHz, CD₃OD): 6 ppm 1.34 (m, 9H) 2.17 (m, 3H) 2.91 (m, 2H) 3.65 (m, 2H) 4.00 (m, 3H) 6.67 (m, 1H) 7.11 (m, 1H) 7.24 (m, 2H) 7.58 (m, 3H) 7.99 (m, 1H) 8.27 (m, 1H). ESI-MS m/z: 462 (M+H)⁺.

### EXAMPLE 14

This example describes the synthesis of 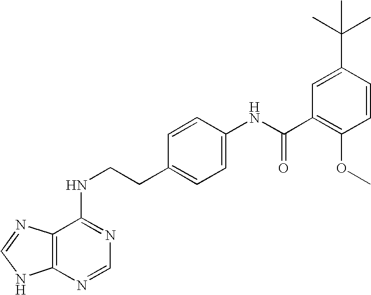
which was prepared according to the protocol below.

The titled was prepared following the procedure of Example 13, but the final coupling was performed with 6-chloropurine instead of compound 18. ¹H NMR (400 MHz, CD₃OD): δ ppm 1.33 (m, 9H) 3.05 (m, 2H) 3.93 (m, 2H) 4.01 (m, 3H) 7.12 (m, 1H) 7.29 (m, 2H) 7.59 (m, 3H) 7.99 (m, 1H) 8.30 (m, 1H) 8.43 (m, 1H). ESI-MS m/z: 445 (M+H)⁺.

### EXAMPLE 15

This example describes the synthesis of the following compounds 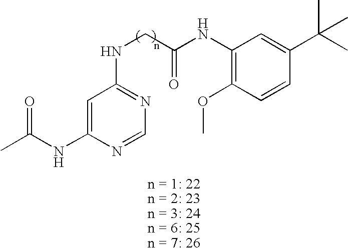
which were prepared according to Example 10 except for the following changes.

Compound 22 was made using amino-acetic acid methyl ester hydrochloride instead of methyl 6-aminohexanoate hydrochloride. ESI-MS m/z: 372 (M+H)⁺.

Compound 23 was made using 3-amino-propionic acid methyl ester hydrochloride instead of methyl 6-aminohexanoate hydrochloride. ESI-MS m/z: 386 (M+H)⁺.

Compound 24 was made using 4-amino-butyric acid methyl ester hydrochloride instead of methyl 6-aminohexanoate hydrochloride. ESI-MS m/z: 400 (M+H)⁺.

Compound 25 was made using 7-amino-heptanoic acid methyl ester hydrochloride instead of methyl 6-aminohexanoate hydrochloride. ESI-MS m/z: 442 (M+H)⁺.

Compound 26 was made using 8-amino-octanoic acid methyl ester hydrochloride instead of methyl 6-aminohexanoate hydrochloride. ESI-MS m/z: 456 (M+H)⁺.

### EXAMPLE 16

This example describes the synthesis of the following compounds 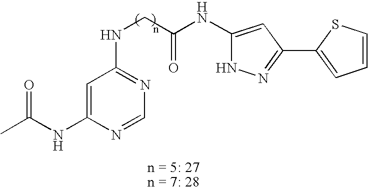
which were prepared according to Example 11 except for the following changes

Compound 27 was made using 6-aminohexanoate hydrochloride was used instead of 7-aminoheptanoic acid methyl ester. ESI-MS m/z: 414 (M+H)⁺.

Compound 28 was made using 8-amino-octanoic acid methyl ester hydrochloride was used in place of 7-aminoheptanoic acid methyl ester. ESI-MS m/z: 442 (M+H)⁺.

### EXAMPLE 17

This example describes the synthesis of 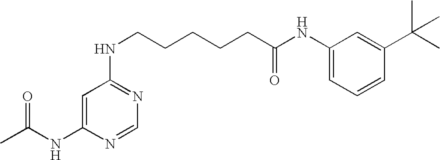
which was prepared according to Example 10 with 3-tert-butyl-phenylamine instead of 5-tert-butyl-o-aniside. ESI-MS m/z: 398 (M+H)⁺.

### EXAMPLE 18

This example describes the synthesis of 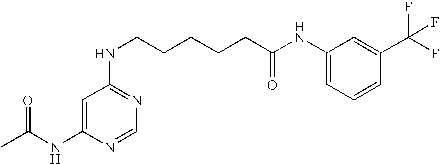
which was prepared according to Example 10 with 3-trifluoromethyl-phenylamine instead of 5-tert-butyl-o-aniside. ESI-MS m/z: 410 (M+H)⁺.

### EXAMPLE 19

This example describes the synthesis of 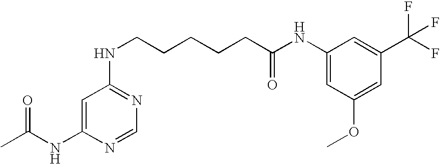
which was prepared according to Example 10 with with 3-methoxy-5-trifluoromethyl-phenylamine instead of 5-tert-butyl-o-aniside. ESI-MS m/z: 440 (M+H)⁺.

### EXAMPLE 20

This example describes the synthesis of 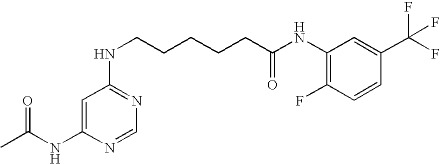
which was prepared according to Example 1 with 2-fluoro-5-trifluoromethyl-phenylamine substituted for 5-tert-butyl-o-aniside. ESI-MS m/z: 428 (M+H)⁺.

### EXAMPLE 21

This example describes the synthesis of 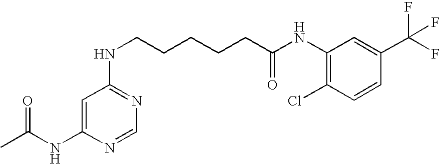
which was prepared according to Example 1 with 2-chloro-5-trifluoromethyl-phenylamine substituted for 5-tert-butyl-o-aniside. ESI-MS m/z: 444 (M+H)⁺.

### EXAMPLE 22

This example describes the synthesis of 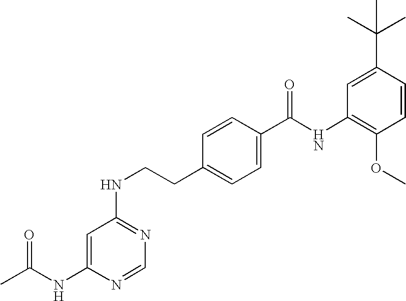
which was prepared according to Example 12 with 5-tert-butyl-o-aniside and 4-(2-tert-butoxycarbonylamino-ethyl)-benzoic acid replacing 5-tert-butyl-2-methoxybenzoic acid and 1-(N-Boc-aminomethyl)-3-(aminomethyl)benzene. ESI-MS m/z: 462 (M+H)⁺.

### EXAMPLE 23

This example describes the synthesis of 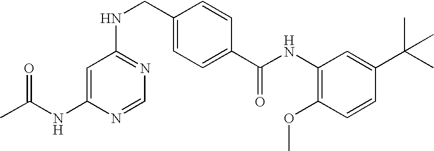
which was prepared according to Example 12 with 5-tert-butyl-o-aniside and 4-(tert-butoxycarbonylamino-methyl)-benzoic acid replacing 5-tert-butyl-2-methoxybenzoic acid and 1-(N-Boc-aminomethyl)-3-(aminomethyl)benzene. ESI-MS m/z: 448 (M+H)⁺.

### EXAMPLE 24

This example describes the synthesis of 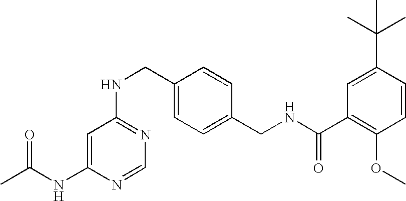
which was prepared according to Example 12 with (4-aminomethyl-benzyl)-carbamic acid tert-butyl ester replacing 1-(N-Boc-aminomethyl)-3-(aminomethyl)benzene. ESI-MS m/z: 462 (M+H)⁺.

The examples described above are set forth solely to assist in the understanding of the invention, and are not intended to limit the scope of the invention in any way.

One skilled in the art will readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the following claims.

1. A method for identifying a ligand binding to an inactive conformation of a target protein kinase, comprising
(a) contacting the inactive conformation of said target protein kinase, which contains or is modified to contain a reactive group at or near a binding site of interest, with one or more ligand candidates capable of covalently bonding to said reactive group thereby forming a kinase-ligand conjugate; and
(b) detecting the formation of said kinase-ligand conjugate and identifying the ligand present in said kinase-ligand conjugate.

2. The method of claim 1 wherein said reactive group is capable of forming a disulfide bond with said ligand candidate.

3. The method of claim 2 wherein said reactive group is a thiol group, masked thiol group, or activated thiol group, which forms a disulfide bond with a thiol functionality present on said ligand candidate.

4. The method of claim 3 wherein said thiol functionality is present as part of a flexible linking group.

5. The method of claim 4 wherein said flexible linking group is of the form —(CH₂)ₙ—S—S—CH₂CH₂NH₂, wherein n is 1 to 5.

6. The method of claim 1 wherein said target protein kinase is contacted with a plurality of said ligand candidates.

7. The method of claim 1 wherein said ligand is less than 1500 daltons in size.

8. The method of claim 1 wherein said ligand is less than 1000 daltons in size.

9. The method of claim 1 wherein said ligand is less than 750 daltons in size.

10. The method of claim 1 wherein said ligand is less than 500 daltons in size.

11. The method of claim 1 wherein said target protein kinase is locked in inactive conformation by alteration of at least one amino acid residue at an inactivation site.

12. The method of claim 11 wherein said alteration is an amino acid substitution.

13. The method of claim 12 wherein an alanine residue is substituted for a wild-type amino acid residue at said inactivation site.

14. The method of claim 11 wherein said inactivation site is selected from the group consisting of the invariant aspartic acid residue in the catalytic loop, the arginine residue in the catalytic loop, the invariant aspartic acid residue in the DFG motif, and the invariant lysine residue in motif II of said target protein kinase.

15. The method of claim 1 wherein said target protein kinase contains said reactive group without further modification.

16. The method of claim 15 wherein said reactive group is a cysteine residue.

17. The method of claim 1 wherein said target protein kinase is modified to contain said reactive group.

18. The method of claim 17 wherein said reactive group is a cysteine residue.

19. The method of claim 2 wherein said target protein kinase and said ligand candidate are contacted in the presence of a reducing agent.

20. The method of claim 19 wherein said reducing agent is 2-mercaptoethanol or cysteamine.

21. The method of claim 1 wherein the formation of said kinase-ligand conjugate is detected by mass spectrometry.

22. The method of claim 21 wherein the kinase-ligand conjugate is subjected directly to mass spectrometry analysis.

23. The method of claim 22 wherein the kinase-ligand conjugate is fragmented prior to mass spectrometry analysis.

24. The method of claim 22 or claim 23 wherein the mass spectrometry analysis also identified the ligand in said conjugate.

25. The method of claim 1 wherein the kinase-ligand conjugate is detected using NMR.

26. The method of claim 25 wherein NMR also identifies the ligand in said conjugate.

27. The method of claim 1 wherein the kinase-ligand conjugate is detected using X-ray crystallography.

28. The method of claim 27 wherein X-ray crystallography also identifies the ligand in said conjugate.

29. The method of claim 1 wherein the kinase-ligand conjugate is detected using capillary electrophoresis.

30. The method of claim 1 wherein the kinase-ligand conjugate is detected using high performance liquid chromatography.

31. The method of claim 1 comprising identifying at least two ligands binding to non-overlapping binding sites of interest of the inactive conformation of said protein kinase.

32. The method of claim 31 further comprising the step of synthesizing a molecule comprising said ligands.

33. A method for identifying a ligand that binds to the inactive conformation of a target protein kinase, comprising
(a) obtaining the inactive conformation of said target protein kinase comprising an —SH group, masked —SH group, or activated —SH group;
(b) combining said inactive conformation of said target protein kinase with one or more ligand candidates wherein said ligand candidates each comprises a disulfide bond;
(c) forming a kinase-ligand conjugate wherein at least one ligand candidate binds to the inactive conformation of the target protein kinase under disulfide exchange conditions, and
(d) detecting the formation of said kinase-ligand conjugate and identifying the ligand present in said conjugate.

34. The method of claim 33 wherein said target protein kinase is locked in an inactive conformation by an amino acid substitution at one or more sites selected from the group consisting of the invariant aspartic acid residue in the catalytic loop, the arginine residue in the catalytic loop, the invariant aspartic acid residue in the DFG motif, and the invariant lysine residue in motif II of said target protein kinase.

35. The method of claim 33 wherein said —SH group, masked —SH group, or activated —SH group is provided by a cysteine residue.

36. The method of claim 35 wherein said target protein kinase is modified to contain a cysteine residue.

37. The method of claim 33 wherein said target protein kinase and said ligand candidate are contacted in the presence of a reducing agent.

38. The method of claim 37 wherein said reducing agent is 2-mercaptoethanol or cysteamine.

39. The method of claim 33 wherein said ligand is a non-peptide small organic molecule, less than 1500 daltons in size.

40. The method of claim 33 wherein said ligand is a non-peptide small organic molecule, less than 1000 daltons in size.

41. The method of claim 33 wherein said ligand is a non-peptide small organic molecule, less than 750 daltons in size.

42. The method of claim 33 wherein said ligand is a non-peptide small organic molecule, less than 500 daltons in size.

43. The method of claim 33 wherein the formation of said kinase-ligand conjugate is detected by mass spectrometry.

44. The method of claim 33 wherein the kinase-ligand conjugate is subjected directly to mass spectrometry analysis.

45. The method of claim 33 wherein the kinase-ligand conjugate is fragmented prior to mass spectrometry analysis.

46. The method of claim 44 or claim 45 wherein the mass spectrometry analysis also identified the ligand in said conjugate.

47. A method for identifying ligands binding to an inactive conformation of a target protein kinase, comprising
(a) contacting the inactive conformation of said protein kinase having a first and a second binding site of interest and containing or modified to contain a nucleophile at or near the first site of interest with a plurality of ligand candidates, said candidates having a functional group reactive with the nucleophile, under conditions such that a reversible covalent bond is formed between the nucleophile and a candidate that has affinity for the first site of interest, to form a kinase-first ligand complex;
(b) identifying the first ligand from the complex of (a);
(c) designing a derivative of the first ligand identified in (b) to provide a small molecule extender (SME) having a first functional group reactive with the nucleophile on the kinase and a second functional group reactive with a second ligand having affinity for the binding second site of interest;
(d) contacting the SME with the kinase to form a kinase-SME complex, and
(e) contacting the kinase-SME complex with a plurality of second ligand candidates, said candidates having a functional group reactive with the SME in said kinase-SME complex, wherein a candidate that has affinity for said second binding site of interest on said kinase forms a reversible covalent bond with said kinase-SME complex, whereby a second ligand is identified.

48. The method of claim 47 wherein said nucleophile is selected from the group consisting of —SH, —OH, —NH₂ and —COOH groups.

49. The method of claim 48 wherein said nucleophile is provided by a side chain of an amino acid residue selected from the group consisting of cysteine, serine, threonine, lysine, asparagine, and glutamine.

50. The method of claim 49 wherein said nucleophile is an —SH group provided by the side chain of a cysteine residue.

51. The method of claim 50 wherein said kinase contains said cysteine residue without further modification.

52. The method of claim 50 wherein said kinase is modified to contain said cysteine residue.

53. The method of claim 50 wherein said SME comprises a group capable of undergoing SN2-like attack or forming a Michael-type adduct with the —SH group of said cysteine residue.

54. The method of claim 53 wherein said group is selected from the group consisting of α-halo acids, fluorophoph(on)ates, epoxides, aziridines, thiiranes, halo-methyl ketones, and halo-methyl amides.

55. The method of claim 50 wherein said second functional group is an —SH group.

56. The method of claim 47 wherein wherein said ligand candidates are members of a small molecule library.

57. The method of claim 56 wherein each member of said library differs in molecular weight from each other member of said library.

58. The method of claim 57 wherein said library contains 1 to 100 members.

59. The method of claim 47 wherein said small molecule extender is selected from the group consisting of 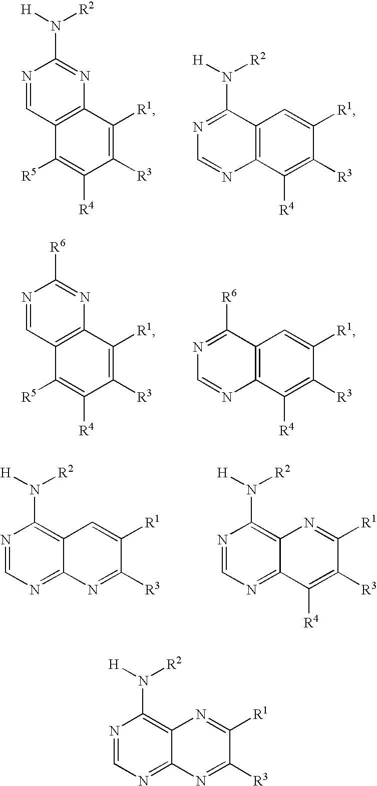
where R¹, R², R³, R⁴, R⁵, and R⁶ are each independently selected from the group consisting of hydrogen, C₁-C₅ alkyl, C₁-C₅ alkylamine, and aryl provided that at least one R group on the SME is a Michael acceptor or —(C═O)CH₂X where X is a halogen and another R group is selected from —(CH₂)ₙ—SR′; —C(═O)—(CH₂)ₙ—SR′; —O—(CH₂)ₙ—SR′; —(CH₂)ₙ—SR′; and a thiol protecting group, wherein R′ is hydrogen or a sulfide and n is 1 to 5.

60. The method of claim 47 wherein said small molecule extender is 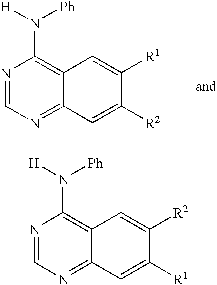
where R¹ is —NHC(═O)CH₂Cl, —NHC(═O)CH═CH₂ or —NHC(═O)CCH and R² is —(CH₂)ₘSSCH₂CH₂NH₂ where m is 1-3.

61. The method of claim 47 wherein said small molecule extender is selected from the group consisting of

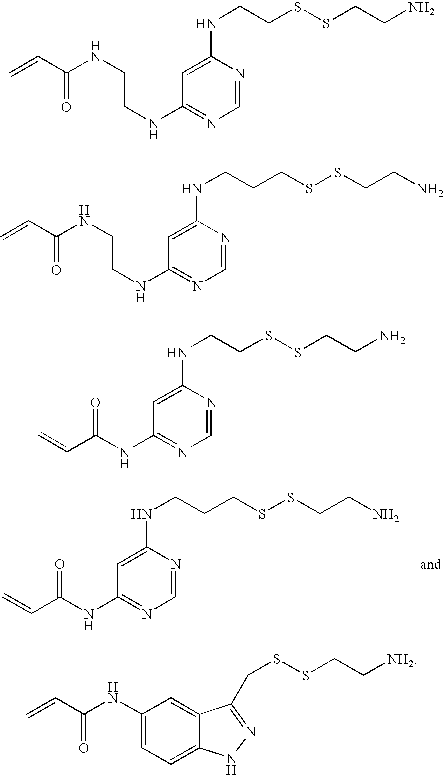

62. A method for identifying ligands binding to an inactive conformation of a target protein kinase, comprising
(a) obtaining the inactive conformation of the target protein kinase comprising an —SH group, masked —SH group, or activated —SH group;
(b) combining in a mixture the inactive conformation of the target protein kinase with a plurality of ligand candidates that are each capable of forming a disulfide bond with the —SH group, masked —SH group, or activated —SH group thereby forming at least one kinase-ligand conjugate;
(c) analyzing the mixture by mass spectrometry; and
(d) detecting the most abundant kinase-ligand conjugate that is formed and identifying the ligand thereon.

63. A method for identifying ligands binding to an inactive conformation of a target protein kinase, comprising
(a) screening a library of ligand candidates with a kinase-ligand conjugate formed by the covalent bonding of the inactive conformation of a kinase comprising a first reactive functionality with a compound that comprises (1) a second reactive functionality and (2) a chemically reactive group, wherein the second reactive functionality of the compound reacts with the first reactive functionality of the inactive conformation of said target protein kinase to form a first covalent bond such that the kinase-ligand conjugate contains a free chemically reactive group, under conditions wherein at least one member of the library forms a second covalent bond with the kinase-ligand conjugate; and
(b) identifying a further ligand that binds covalently to the chemically reactive group of the kinase-ligand conjugate.
